(19) United States
(12) Patent Application Publication
(10) Pub. No.: US 2011/0028719 A1
(43) Pub. Date: Feb. 3, 2011

(54) SCREENING METHODS FOR AMYLOID BETA MODULATORS

(76) Inventor: Jacek Slon-Usakiewicz, Milton (CA)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/301,155

(22) PCT Filed: May 18, 2007
(86) PCT No.: PCT/CA07/00900

§ 371 Date: Sep. 30, 2010

Related U.S. Application Data
(60) Provisional application No. 60/801,848, filed on May 19, 2006.
(60) Provisional application No. 60/851,256, filed on Oct. 12, 2006.
(60) Provisional application No. 60/858,548, filed on Nov. 13, 2006.

(51) Int. Cl. C40B 30/04; C07D 295/12

(52) U.S. Cl. 544/177; 506/9

(57) ABSTRACT

The present invention relates to methods for screening, identifying, and/or quantifying modulators of amyloid and/or aggregates, fibrils or components thereof, in particular modulators of amyloid β-peptide (Aβ) or Aβ fibrils. Aspects of the invention provide methods for screening putative modulators against an Amyloid target, in particular an Aβ target, so as to determine which modulators bind to or interact with the target, or interfere with the interaction of an indicator agent and the target. Particular aspects of the invention employ mass spectrometric methods for the screening of an Amyloid target against compound libraries, in particular mixtures of compounds or combinatorial libraries.

### FIELD OF THE INVENTION

The invention relates to a method for screening, identifying, and/or quantifying modulators of amyloid and/or aggregates, fibrils or components thereof, in particular amyloid β-peptide (Aβ) or Aβ fibrils.

### BACKGROUND OF THE INVENTION

Multiple lines of evidence suggest that the accumulation of neurotoxic oligomeric/protofibrillar aggregates of amyloid β-peptide (Aβ) is a central event in the pathogenesis of Alzheimer's disease (AD) [Selkoe, D. J., J. Clin Invest. 110, 1375-1381 (2002) and Wong, P. C., et al, Nat. Neurosci. 5, 633-639 (2002]. This has led to attempts to develop therapies based upon blocking the generation of Aβ (e.g., with β- or γ-secretase inhibitors), accelerating its removal, or preventing its aggregation and toxicity. The potential utility of anti-Aβ therapies for Aβ has received tentative support from a clinical trial of a vaccine, which suggested clinical and neuropathological improvement in a small cohort of AD patients [Nicoll, J. A. R., et al, Nat. Med. 9, 448-452 (2003) and Hock C. et al., Neuron 38, 547-554 (2003}]. However, the anti-Aβ vaccine also induced a T-cell-mediated meningo-encephalitis in some patients which renders this particular vaccine unsuitable for widespread clinical use [Orgogozo, J. M., et al, Neurology 61, 46-54 (2003)]. Nevertheless, Aβ vaccines have been shown in some mouse models to act via antibody-mediated inhibition of Aβ fibrillogenesis and toxicity [Schenk D, et al., Nature 400, 173-177 (1999); McLaurin, J. et al., Nat. Med. 8, 1263-1269 (2002); and Golde, T. E. J. Clin. Invest. 111, 11-18 (2003)].

### SUMMARY OF THE INVENTION

Broadly stated the present invention relates to methods for screening, identifying, and/or quantifying modulators of amyloid and/or aggregates, fibrils or components thereof, in particular modulators of Aβ-peptide (Aβ) or Aβ fibrils.

In an aspect, the invention provides a method for screening putative modulators against an Amyloid target, in particular an Aβ target, so as to determine which modulators bind to or interact with the Amyloid target, in particular an Aβ target. The invention further provides a method for the determination of the structure of those putative modulators that bind to or interact with the Amyloid target, in particular an Aβ target.

The invention also relates to a method of ascertaining the specificity and affinity of putative modulators, especially small organic molecules, to bind to or interact with Amyloid targets, in particular Aβ targets.

In another aspect, the invention provides a method for determining the relative or absolute binding affinity or thermodynamic dissociation constant (K_{d}) of putative modulators that bind to or interact with an Amyloid target, in particular an Aβ target. Further, the invention provides a method for determining the absolute binding affinity or dissociation constant of putative modulators that bind to or interact with an Amyloid target, in particular an Aβ target. Still further, the invention relates to methods for the determination of the structure of putative modulators that bind to an Amyloid target, in particular an Aβ target.

In another aspect, the invention relates to methods for determining the binding specificity of a putative modulator for an Amyloid target, in particular an Aβ target, compared to a control. Thus, the present invention facilitates the determination of selective modulators and the elimination of non-specific modulators of amyloid, in particular Aβ, from further consideration for drug discovery efforts. The invention relates to methods for the determination of the structure of selective modulators.

In aspects, the invention utilizes an immobilized Amyloid target, in particular an Aβ target, for analysis of amyloid modulators, in particular Aβ modulators. In a particular aspect, the invention provides a method for determining the relative binding affinity of putative modulators for an Amyloid target, in particular an Aβ target, comprising contacting the putative modulators with an Amyloid target, in particular an Aβ target, immobilized onto a support and detecting the breakthrough volume of the putative modulators.

Mass spectrometric methods can be employed for the screening of an Amyloid target, in particular an Aβ target, against compound libraries, in particular mixtures of compounds or combinatorial libraries. This screening procedure may be facilitated by the combined power of mass spectrometric methods and the screening methods performed. Therefore, the invention provides methods for screening for modulators of amyloid, in particular Aβ, through the use of mass spectrometry (MS). In an aspect, the invention provides a method for MS-based determination of the relative or absolute binding affinity of putative modulators for an Amyloid target, in particular an Aβ target. In another aspect, the invention provides methods for identifying modulators of amyloid, in particular Aβ, by determining the relative affinity of putative modulators for an Amyloid target, in particular an Aβ target, using MS.

In accordance with aspects of the invention, an Amyloid target, in particular an Aβ target, is presented with one or more putative modulators under conditions such that interaction or binding of the putative modulators to the target can occur. The resulting complex, which may be of one or even hundreds of individual complexes of the putative modulators and target is then subjected to mass spectrometric evaluation in accordance with the invention.

In a particular aspect, preparative mass spectrometry is employed to isolate individual complexes which can then be fragmented under controlled conditions within the mass spectrometric environment for subsequent analysis. In this way, the nature and degree, or absolute binding affinity, of the binding of the putative modulators to an Amyloid target, in particular an Aβ target, can be ascertained. Identification of specific and strong affinity modulators can be made and compounds can be selected for use as therapeutics, or as lead compounds for subsequent modification into improved forms for therapeutic uses.

In some aspects the invention utilizes the concept of providing an insolubilized target for analysis of modulators by MS. In particular aspects, the invention provides a method for determining the relative binding affinity of putative modulators for an Amyloid target, in particular an Aβ target, comprising contacting putative modulators with an Amyloid target, in particular an Aβ target, immobilized onto a support and detecting the breakthrough volume of the putative modulators by mass spectrometry.

In an aspect of the invention, modulators of amyloid, in particular Aβ, are determined using a combination of frontal affinity chromatography (FAC) with mass spectrometry (MS) (“FAC-MS”) to screen putative modulators to identify and rank putative modulators that bind to or interact with an Amyloid target, in particular an Aβ target. Thus, the present invention provides methods for screening compound libraries using frontal affinity chromatography in combination with mass spectrometry. In this aspect, an Amyloid target, in particular an Aβ target, is generally immobilized on a suitable support and the putative modulators are continuously contacted with the immobilized target. Putative modulators will bind to the target with differing affinities. Depending on their affinity, individual putative modulators are retained on the support causing an increase in their breakthrough volume. Once a putative modulator begins eluting it is continually present in the effluent. Putative modulators having little or no affinity for the Amyloid target, in particular Aβ target, breakthrough earlier in the effluent compared to putative modulators having a higher affinity for the target.

In aspects of the invention, MS is employed to continuously or intermittently monitor the frontal affinity chromatography effluent. Using MS, the identity and breakthrough time for each putative modulator on the column can be determined. FAC-MS allows the relative or absolute affinity of each member of a compound library for the Amyloid target, in particular an Aβ target, to be determined relative to other members of the compound library under binding conditions. Using methods of the present invention, an accurate ranking of the relative affinity of putative modulators for an Amyloid target, in particular an Aβ target, can be ascertained.

In one of its aspects, the present invention is directed to a method for determining the relative affinity of a plurality of putative modulators to an Amyloid target, in particular an Aβ target, which comprises:
(a) providing a plurality of putative modulators of amyloid, in particular Aβ,
(b) continuously applying the modulators, under frontal affinity chromatography conditions, to a column comprising an Amyloid target, in particular an Aβ target, optionally immobilized, whereby the target is continuously contacted with the putative modulators to provide an effluent;
(c) continuously or intermittently applying the effluent to a mass spectrometer to provide mass spectra of the constituent putative modulators present in the effluent; and
(d) evaluating the mass spectra to determine a breakthrough time for each of the putative modulators.

In a preferred embodiment, the above method further comprises the step of
(e) determining an affinity to the Amyloid target, in particular Aβ target, for a putative modulator relative to another putative modulator by comparing the breakthrough time on the column for the putative modulator relative to the other putative modulator.

In another preferred embodiment, the above method further comprises the step of:
(f) determining a dissociation constant, K_{d}, for a putative modulator and the Amyloid target, in particular Aβ target.

The putative modulators may comprise individual compounds, mixtures of compounds, or compound libraries. Compound libraries may be generated or obtained by any means including, by way of example, combinatorial chemistry techniques or from fermentation broths, plant extracts, cellular extracts and the like. A compound library employed in a method of the invention may comprise less than about 50,000, 25,000, 20,000, 15,000, 10000, 5000, 1000, 500 or 100 putative modulators, in particular from about 5 to about 100, 5 to about 200, 5 to about 300, 5 to about 400, 5 to about 500, 10 to about 100, 10 to about 200, 10 to about 300, 10 to about 400, 10 to about 500, 10 to bout 1000, 20 to about 100, 20 to about 200, 20 to about 300, 20 to about 400, 20 to about 500, 20 to about 1000, 50 to about 100, 50 to about 200, 50 to about 300, 50 to about 400, 50 to about 500, 50 to about 1000, 100 to about 200, 100 to about 300, 100 to about 400, 100 to about 500, 100 to about 1000, 200 to about 300, 200 to about 400, 200 to about 500, 200 to about 1000, 300 to about 500, 300 to about 1000, 300 to 2000, 300 to 3000, 300 to 5000, 300 to 6000, 300 to 10,000, 500 to about 1000, 500 to about 2000, 500 to about 3000, 500 to about 5000, 500 to about 6000, or 500 to about 10,000 putative modulators.

In one embodiment of this invention, a compound library is employed that independently comprises putative modulators selected from the group consisting of carbohydrates, monosaccharides, oligosaccharides, polysaccharides, amino acids, peptides, oligopeptides, polypeptides, proteins, nucleosides, nucleotides, oligonucleotides, polynucleotides, lipids, steroids, glycopeptides, glycoproteins, proteoglycans, synthetic analogs or derivatives thereof, and the like.

In another embodiment, a compound library is employed that comprises synthetic small molecule organic compounds.

In aspects of the invention, the Amyloid target is an Aβ target in particular Aβ oligomers, aggregated Aβ or Aβ fibrils. In particular aspects, the Aβ target is Aβ fibrils.

In aspects of the invention, an amyloid target is immobilized, in particular an amyloid target is immobilized on or bound to a solid phase support. In particular aspects, the target is directly or indirectly covalently bound to a solid phase support. In more particular embodiments, the solid phase support comprises resin beads, glass beads, silica chips, silica capillaries or agarose. In aspects, a solid phase support in the form of a column is employed comprising from about 1 pmol to about 10 nmol of amyloid target sites.

In aspects of methods of the invention, the effluent from a column is diluted with a supplemental diluent before analysis by mass spectrometry.

In particular aspects of the invention, the mass spectrometer employed is an electrospray mass spectrometer.

In FAC-MS aspects of the invention, constant effluent monitoring is typically not required since the putative modulators continuously elute under frontal chromatography conditions once they breakthrough the column. Therefore, the invention contemplates methods employing a plurality of FAC-MS analyses conducted simultaneously using a single mass spectrometer to intermittently monitor each column.

Accordingly, in another of its aspects, this invention provides a method for screening a plurality of compound libraries to determine the relative affinity of a plurality of putative modulators in each library to an Amyloid target, in particular an Aβ target, comprising:
(a) providing a plurality of compound libraries, each library comprising a plurality of putative modulators;
(b) continuously applying each compound library to a separate column comprising an Amyloid target, in particular an Aβ target, optionally immobilized (e.g., bound to a solid phase support) under frontal affinity chromatography conditions whereby the target is continuously contacted with the compound library to provide an effluent from each column;
(c) intermittently applying the effluent from each column to a mass spectrometer to provide mass spectra of the constituent putative modulators present in the effluent; and
(d) evaluating the mass spectra to determine a breakthrough time for each of the putative modulators in each compound library.

In an embodiment, the above method further comprises:
(e) determining an affinity to the Amyloid target, in particular an Aβ target, for a putative modulator in each library relative to another putative modulator(s) in the same library by comparing the breakthrough time on the column for the putative modulator relative to the other putative modulator(s) in the same library.

In another embodiment, the above method further comprises:
(f) determining a dissociation constant, K_{d}, for a putative modulator in a compound library and the Amyloid target, in particular an Aβ target.

In particular embodiments employing a plurality of columns with insolubilized targets, from about 2 to about 200, about 2 to about 150, about 2 to about 100 columns, about 2 to about 75 columns, about 2 to about 50 columns, about 2 to about 25, about 2 to about 20, about 2 to about 15, or about 2 to about 10 columns, may be employed.

Accordingly, in another aspect, this invention provides a method for screening a compound library to determine the relative affinity of a plurality of putative modulators to an Amyloid target, in particular an Aβ target, relative to one or more indicator agents which method comprises:
(a) providing a compound library comprising a plurality of putative modulators,
(b) continuously applying the compound library to a column comprising an Amyloid target, in particular an Aβ target, optionally immobilized (e.g., bound to a solid phase support), under frontal affinity chromatography conditions to equilibrate the column with the compound library;
(c) providing at least one indicator agent having a pre-determined affinity for the target, and having a pre-determined breakthrough time on the column in the absence of the compound library;
(d) continuously applying (i) a mixture comprising the compound library and the indicator agent, or (ii) the indicator agent, to the column under frontal affinity chromatography conditions to provide an effluent; and
(e) analyzing the effluent by mass spectrometry to determine a breakthrough time for the indicator agent.

In an embodiment, the above method further comprises:
(f) determining whether any putative modulators disrupt binding of the indicator agent to the target, or have an affinity for the target greater than the indicator agent by comparing the breakthrough time for an indicator agent in the presence of the compound library with the pre-determined breakthrough time for the indicator agent in the absence of the compound library.

This invention provides a method for screening a compound library to determine if any member of the library interferes with the interaction of the indicator agent and the Amyloid target, in particular an Aβ target, or has an affinity for an Amyloid target, in particular an Aβ target, higher than a pre-selected indicator agent. Using this embodiment, putative modulators (e.g. compound libraries) can be rapidly screened to identify those putative modulators having a pre-determined minimum level of affinity for the target.

In an aspect, this invention provides a method for screening a compound library for putative modulators that interfere with the interaction of an indicator agent and an Amyloid target, in particular an Aβ target, or breaks down the target, which method comprises:
(a) providing a compound library comprising a plurality of putative modulators,
(b) continuously applying the compound library to a column comprising an Amyloid target, in particular an Aβ target, optionally immobilized (e.g., bound to a solid phase support), under frontal affinity chromatography conditions to equilibrate the column with the compound library;
(c) providing at least one indicator agent having a pre-determined affinity for the target, and having a pre-determined breakthrough time on the column in the absence of the compound library;
(d) continuously applying (i) a mixture comprising the compound library and the indicator agent, or (ii) the indicator agent, to the column under frontal affinity chromatography conditions to provide an effluent; and
(e) analyzing the effluent by mass spectrometry to determine a breakthrough time for the indicator agent in the presence and absence of the compound library.

In an embodiment, the above method further comprises:
(f) determining whether any putative modulators interfere with the interaction or binding of the indicator agent to the target or breaks down the target, by comparing the breakthrough time for an indicator agent in the presence of the compound library with the pre-determined breakthrough time for the indicator agent in the absence of the compound library.

In embodiments of the invention employing an indicator agent, a compound library may comprise less than about 50,000, 25,000, 20,000, 15,000, 10,000, 5,000, 1000, 500, or 100 putative modulators. In particular embodiments of the invention, a compound library comprises about 5 to about 100, about 5 to about 200, about 5 to 250, about 5 to about 300, about 5 to about 400, about 5 to about 500, about 10 to about 100, about 10 to about 200, about 10 to about 300, about 10 to about 400, about 10 to about 500, about 10 to bout 1000, about 20 to about 100, about 20 to about 200, about 20 to about 300, about 20 to about 400, about 20 to about 500, about 20 to about 1000, about 50 to about 100, about 50 to about 200, about 50 to about 300, about 50 to about 400, about 50 to about 500, about 50 to about 1000, about 100 to about 200, about 100 to about 300, about 100 to about 400, about 100 to about 500, about 100 to about 1000, about 200 to about 300, about 200 to about 400, about 200 to about 500, about 200 to about 1000, about 300 to about 500, about 300 to about 1000, about 300 to 2000, about 300 to 3000, about 300 to 5000, about 300 to 6000, about 300 to 10,000, about 500 to about 1000, about 500 to about 2000, about 500 to about 3000, about 500 to about 5000, about 500 to about 6000, or about 500 to about 10,000 putative modulators.

In particular embodiments of this method of the invention, an indicator agent has a pre-determined breakthrough time in the absence of the compound library of less than about 30, 20, 15, 10, 8, 6, 5, 4, 3, 2, or 1 minutes, in particular less than about 15, 10, 5, or 1 minutes.

In other particular embodiments of the invention, the indicator agent is an amyloid, in particular an amyloid monomer or oligomer, more particularly a β-amyloid monomer, most particularly an Aβ1-42 monomer.

In particular embodiments, where the Amyloid target is an Aβ target, in particular Aβ fibrils, and the indicator agent is an Aβ1-42 monomer, a putative modulator shifts the break through time of the indicator agent by at least 1 to 95%, 1 to 90%, 1 to 80%, 1 to 75%, 1 to 50%, 1 to 25%, 2 to 90%, 5 to 90%, 5 to 80%, 5 to 75%, 5 to 60%, 5 to 50%, 5 to 40%, 5 to 30%, 5 to 25%, 5 to 20%, 5 to 15%, 5 to 15%, or 5 to 10%.

The invention also contemplates methods employing indicator agents and a plurality of compound libraries. Accordingly, in aspects of the present invention, a method is provided for screening a plurality of compound libraries to determine the relative affinity of a plurality of putative modulators to an Amyloid target, in particular an Aβ target, relative to one or more indicator agents comprising:
(a) providing a plurality of compound libraries comprising a plurality of putative modulators,
(b) continuously applying each compound library to a separate column comprising an Amyloid target, in particular an Aβ target, optionally immobilized (e.g, bound to a solid phase support), under frontal affinity chromatography conditions to equilibrate the column with the compound library;
(c) providing at least one indicator agent having a pre-determined affinity for the Amyloid target, in particular an Aβ target, and having a pre-determined breakthrough time on each column in the absence of the compound library;
(d) continuously applying (i) a mixture comprising the compound library and the indicator agent, or (ii) the indicator agent, to each column under frontal affinity chromatography conditions to provide an effluent; and
(e) analyzing the effluent from each column by mass spectrometry to determine a breakthrough time for the indicator agent.

In an embodiment, the above method further comprises the step of:
(f) determining whether any putative modulators of a compound library disrupt binding of the indicator agent to the Amyloid target, in particular an Aβ target, or have an affinity for the target greater than the indicator agent by comparing the breakthrough time for the indicator agent in the presence of the compound library with the pre-determined breakthrough time for the indicator agent in the absence of the compound library.

In other aspects of the present invention, a method is provided for screening a plurality of compound libraries for putative modulators that interfere with the interaction of the indicator agent and an Amyloid target, in particular an Aβ target, comprising:
(a) providing a plurality of compound libraries comprising a plurality of putative modulators,
(b) continuously applying each compound library to a separate column comprising an Amyloid target, in particular an Aβ target, optionally immobilized (e.g, bound to a solid phase support), under frontal affinity chromatography conditions to equilibrate the column with the compound library;
(c) providing at least one indicator agent having a pre-determined affinity for the target, and having a pre-determined breakthrough time on each column in the absence of the compound library;
(d) continuously applying (i) a mixture comprising the compound library and the indicator agent, or (ii) the indicator agent, to each column under frontal affinity chromatography conditions to provide an effluent; and
(e) analyzing the effluent from each column by mass spectrometry to determine a breakthrough time for the indicator agent.

In an embodiment, the above method further comprises the step of:
(f) determining whether any putative modulators of a compound library interfere with the interaction of the indicator agent and an Amyloid target, in particular an Aβ target, by comparing the breakthrough time for the indicator agent in the presence of the compound library with the pre-determined breakthrough time for the indicator agent in the absence of the compound library.

In particular embodiments of this method of the invention, an indicator agent has a pre-determined breakthrough time in the absence of the compound library of less than about 30, 20, 15, 10, 8, 6, 5, 4, 3, 2, or 1 minutes, in particular less than about 15, 10, 5 or 1 minutes.

In other particular embodiments of the invention, the indicator agent is an amyloid, in particular an amyloid monomer or oligomer, more particularly a β-amyloid monomer, most particularly an Aβ1-42 monomer.

In particular embodiments, where the Amyloid target is an Aβ target, in particular Aβ fibrils, and the indicator agent is an Aβ1-42 monomer, a putative modulator shifts the break-through time of the indicator agent by at least 1 to 95%, 1 to 90%, 1 to 80%, 1 to 75%, 1 to 50%, 1 to 25%, 2 to 90%, 5 to 90%, 5 to 80%, 5 to 75%, 5 to 60%, 5 to 50%, 5 to 40%, 5 to 30%, 5 to 25%, 5 to 20%, 5 to 15%, 5 to 15%, or 5 to 10%.

In an aspect, the invention provides a method for screening a compound library to determine the relative affinity of a plurality of putative modulators to an Amyloid target or a plurality of Amyloid targets relative to an indicator agent or a plurality of indicator agents, which method comprises:
(a) providing a compound library comprising a plurality of putative modulators;
(b) providing at least one void marker compound;
(c) providing an indicator agent or a plurality of indicator agents for an Amyloid target, each indicator agent having a pre-determined affinity for the Amyloid target and having a pre-determined breakthrough time on the column in the absence of the compound library relative to a void marker compound;
(d) applying the compound library to a column comprising an Amyloid target or a plurality of Amyloid targets, each Amyloid target optionally bound to a solid phase support, under frontal affinity chromatography conditions to equilibrate or partially equilibrate the column with the compound library;
(e) applying (i) a mixture comprising the compound library, the void marker compound and the indicator agents or compounds, or (ii) the void marker compound and the indicator agents or compounds, to the column under frontal affinity chromatography to provide an effluent; and
(f) analyzing the effluent to determine a breakthrough time for the indicator agent or compounds.

In an embodiment, the above method further comprises the step of: (g) determining whether any putative modulators of the compound library have an affinity for the target greater than the indicator agent by comparing the breakthrough time for the indicator agent from step (f) with the pre-determined breakthrough time for the indicator agent in the absence of the compound library.

In another aspect, the invention provides a method for screening a plurality of compound libraries to determine the relative affinity of a plurality of putative modulators to an Amyloid target or a plurality of Amyloid targets relative to an indicator agent or a plurality of indicator agents, which comprises:
(a) providing a plurality of compound libraries comprising a plurality of putative modulators;
(b) providing at least one void marker compound;
(c) providing an indicator agent or a plurality of indicator agents for each Amyloid target, each indicator agent having a predetermined affinity for the Amyloid target and having a pre-determined breakthrough time on each column in the absence of the compound library relative to a void marker compound;
(d) applying each compound library to a separate column comprising an Amyloid target or a plurality of Amyloid targets, each Amyloid target optionally bound to a solid phase support, under frontal affinity chromatography conditions to equilibrate or partially equilibrate the column with the compound library;
(e) applying (i) a mixture comprising the compound library, the void marker compound and the indicator agent or compounds, or (ii) the void marker and the indicator agent or compounds, to each column under frontal affinity chromatography conditions to provide an effluent;
(f) analyzing the effluent from each column to determine a breakthrough time for the indicator agent or compounds.

In an embodiment, the above method further comprises the step of: (g) determining whether any putative modulators of a compound library have an affinity for an the target greater than the indicator agent by comparing the breakthrough time for the indicator agent from step (f) with the pre-determined breakthrough time for the indicator agent in the absence of the compound library.

In an aspect, the invention provides a method for screening a compound library to determine the relative affinity of a plurality of putative modulators to an Amyloid target relative to an indicator agent having a pre-determined affinity for the Amyloid target, which method comprises:
(a) providing a compound library comprising a plurality of putative modulators;
(b) providing at least one void marker compound;
(c) providing a column comprising an Amyloid target optionally bound to a solid phase support;
(d) providing an indicator agent having a pre-determined affinity for the target and having a pre-determined breakthrough time on each column in the absence of the compound library relative to a void marker compound and having a pre-determined signal intensity in the presence of the compound library;
(e) applying a mixture comprising the compound library and the indicator agent to the column under frontal affinity chromatography conditions to provide an effluent; and
(f) analyzing the effluent to determine a breakthrough time and/or signal intensity for the indicator agent.

In an embodiment, the above method further comprises the step of: (g) determining whether any putative modulators of the compound library have an affinity for an the target by comparing the breakthrough time for the indicator agent from step (f) with the pre-determined breakthrough time for the indicator agent in the absence of the compound library.

In another embodiment, the above method further comprises the step of: (h) determining whether the affinity for the target is due to a plurality of modulators having weaker affinity for the target relative to the indicator agent or to one or more putative modulators having stronger affinity for the target relative to the indicator agent by comparing the signal intensity of the indicator agent in the effluent with the pre-determined signal intensity for the indicator agent.

In particular embodiments of this method of the invention, an indicator agent has a pre-determined breakthrough time in the absence of the compound library of less than about 30, 20, 15, 10, 8, 6, 5, 4, 3, 2, or 1 minutes, in particular less than about 15, 10, 5 or 1 minutes.

In other particular embodiments of this method of the invention, the indicator agent is an amyloid, in particular an amyloid monomer or oligomer, more particularly a β-amyloid monomer, most particularly an Aβ 1-42 monomer.

The methods of this invention can be used for the rapid screening of large collections of putative modulators. It is also possible to screen mixtures of large numbers of compounds that are generated via combinatorial or other means. When a large mixture of compounds is exposed to an Amyloid target, in particular an Aβ target, a small fraction of putative modulators may exhibit some binding affinity to the target or disrupt binding of an indicator agent to the target (e.g., shift the breakthrough time of an indicator agent). The actual number of putative modulators that bind an Amyloid target or disrupt the binding of an indicator agent to the Amyloid target may be based on the concentration of the target, the relative concentrations of the components of the combinatorial mixture, and the absolute and relative binding affinities of these components.

A method of the invention may further comprise determining the structure of a putative modulator identified according to the method. Therefore, the invention also contemplates a modulator of amyloid, in particular Aβ identified according to a method of the invention.

Another aspect of the present invention provides a method of conducting a drug discovery business comprising:
(a) providing one or more methods for identifying putative modulators of amyloid, in particular Aβ;
(b) conducting therapeutic profiling of putative modulators identified in step (a), or further analogs thereof, for efficacy and toxicity in in vitro assays (e.g. cell based assays) or in animals; and
(c) formulating a pharmaceutical composition including one or more modulators identified in step (b) as having an acceptable therapeutic profile.

In certain aspects, the subject method can also include a step of establishing a distribution system for distributing the pharmaceutical composition for sale, and may optionally include establishing a sales group for marketing the pharmaceutical composition.

This invention therefore contemplates a pharmaceutical composition comprising one or more amyloid modulators identified according to a method of the invention and a pharmaceutically acceptable carrier, excipient or vehicle. The invention also contemplates the use of a modulator or pharmaceutical composition of the invention in the preparation of a medicament to treat a disease disclosed herein. The invention further contemplates administering a modulator or pharmaceutical composition of the invention to a subject in need thereof, in particular to a subject with a disease disclosed herein.

The invention also contemplates kits for carrying out the methods of the invention. Such kits typically comprise two or more components required for performing a method of the invention including without limitation compounds, reagents, containers, and/or equipment.

These and other aspects, features, and advantages of the present invention should be apparent to those skilled in the art from the following drawings and detailed description.

### BRIEF DESCRIPTION OF THE DRAWINGS

The invention will now be described in relation to the drawings in which:

FIG. 1 is a chromatogram showing FAC-MS analysis of the binding of free Aβ monomer to immobilized Aβ fibrils.

FIG. 2 is a chromatogram showing FAC-MS analysis of the binding of free Aβ monomer to immobilized Aβ fibrils in the presence of a scyllo-cyclohexanehexyl compound (AZD-103, scyllo-inositol).

FIG. 3 is a graph showing the dose dependent reduction in free Aβ monomer binding to immobilized Aβ fibrils in the presence of a scyllo-cyclohexanehexyl compound (AZD-103).

FIG. 4 is a graph of the FAC-MS % shift results of the free Aβ monomer assayed with immobilized Aβ fibrils in the presence of various cyclohexanehexyls at 1 and 10 μM.

FIG. 5 is a graph showing the effects of pretreatment of a column of immobilized Aβ fibrils with AZD-103.

FIG. 6 is a bar graph showing the FAC-MS % shift of free amyloid-β1-42 monomer binding to immobilized Aβ fibrils in the presence of AZD-103, chiro-inositol, D-pinitol, myo-inositol, 1,2,5,6-bis-O-(1-Methylethyldene)-3-methyl-1D-chiro-inositol, 1,2,3,4-DL-β-cyclohexyldene-5-O-methyl-L-chiro-inositol, D-Myo-inositol-2,4-bis-phosphate, 2-O-b-L-Arabinopyranosyl myo-inositol, and myo-inositol hexasulfate hexapotasium.

FIG. 7 is a bar graph showing the FAC-MS % shift of free amyloid-β1-42 monomer binding to immobilized Aβ fibrils in the presence of AZD-103, Kasugamycin hydrochloride, Conduritol B epoxide, Chlorogenic acid, 1R,3R,4R,5R-Quinic acid, Streptomycin sulfate, and Phytic acid.

FIG. 8 is a bar graph showing the FAC-MS % shift of free amyloid-β1-42 monomer binding to immobilized Aβ fibrils in the presence of AZD-103, 2,3,4,5,6-pentakis[(2,2-dimethylpropanoyl)oxy]cyclohexyl pivalate, 2,3,4,5-tetrakis[(2,2-dimethylpropanoyl)oxy]-6-hydroxycyclohexyl pivalate, 2,5-bis(acetoxy)-3,4,6-tris[(2,2-dimethylpropanoyl)oxy]cyclohexyl pivalate, 2,3,5,6-tatrakis(benzoyloxy)-4-hydroxycyclohexyl benzoate, 2,3,4,5,6-pentakis(isobutyryloxy)cyclohexyl 2-methylpropanoate, 2-hydroxy-3,4,5,6-tetrakis(isobutyryloxy)cyclohexyl 2-methylpropanoate, 2,3,4,5,6-pentakis(propionyloxy)cyclohexyl propionate, 2-hydroxy-3-4,5,6-tetrakis[(3-methylbutanoyl)oxo]cyclohexyl 3-methylbutanoate, and 2-(acetyloxy)-3,4,5,6-tetrakis[(3-methylbutanoyl)oxy]cyclohexanyl 3-methylbutanoate

### DETAILED DESCRIPTION OF THE INVENTION

Unless defined otherwise, all technical and scientific terms used herein have the same meaning as commonly understood by one of ordinary skill in the art to which this invention belongs.

### GLOSSARY

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “a compound” includes a mixture of two or more compounds.

“Breakthrough time” refers to the period of time between elution of the void volume and the front corresponding to the elution of a particular compound (e.g., putative modulator or indicator agent) during frontal affinity chromatography with mass spectroscopy detection.

“Breakthrough volume” refers to the effluent volume passing through the column that allows the output agent concentration to equal the input test agent concentration.

The term “compound library” refers to a mixture or collection of one or more putative modulators generated or obtained in any manner. Any type of molecule that is capable of interacting, binding or has affinity for an Amyloid target, in particular an Aβ target, may be present in the compound library. For example, compound libraries screened using this invention may contain naturally-occurring molecules, such as carbohydrates, monosaccharides, oligosaccharides, polysaccharides, amino acids, peptides, oligopeptides, polypeptides, proteins, receptors, nucleic acids, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such as peptidomimetics and the like; and non-naturally occurring molecules, such as “small molecule” organic compounds generated, for example, using combinatorial chemistry techniques; and mixtures thereof. In aspects of the invention, a compound library comprises cyclohexane polyalcohol compounds and/or derivatives thereof.

A library typically contains more than one putative modulator or member, i.e., a plurality of members or putative modulators. In aspects of the invention, a compound library may comprise less than about 50,000, 25,000, 20,000, 15,000, 10000, 5000, 1000, 500 or 100 putative modulators, in particular from about 5 to about 100, 5 to about 200, 5 to about 300, 5 to about 400, 5 to about 500, 10 to about 100, 10 to about 200, 10 to about 300, 10 to about 400, 10 to about 500, 10 to bout 1000, 20 to about 100, 20 to about 200, 20 to about 300, 20 to about 400, 20 to about 500, 20 to about 1000, 50 to about 100, 50 to about 200, 50 to about 300, 50 to about 400, 50 to about 500, 50 to about 1000, 100 to about 200, 100 to about 300, 100 to about 400, 100 to about 500, 100 to about 1000, 200 to about 300, 200 to about 400, 200 to about 500, 200 to about 1000, 300 to about 500, 300 to about 1000, 300 to 2000, 300 to 3000, 300 to 5000, 300 to 6000, 300 to 10,000, 500 to about 1000, 500 to about 2000, 500 to about 3000, 500 to about 5000, 500 to about 6000, or 500 to about 10,000 putative modulators. In particular aspects, a compound library may comprise less than about 50,000, 25,000, 20,000, 15,000, 10,000, 5,000, 1000, or 500 putative modulators. In particular embodiments a compound library comprises about 5 to about 5000, 20 to about 5000, 50 to about 1000, 5 to about 500, 5 to about 250, 5 to about 100, 50 to about 100, or 5 to about 50 putative modulators. When an indicator agent is employed, a compound library may contain less than about 50,000 members, preferably, less than about 10,000, 5000, 2500, 1000, or 500 members. When an indicator agent is not employed, a compound library may contain less than about 10,000 members; preferably, from about 1 to about 1,000 or about 1 to about 500 members; and more preferably, from about 5 to about 100 members. In some aspects, the members of a compound library have molecular weights less than about 10000 DA, 8000 DA, 7000 DA, 5000 Da, 2500 Da, 2000 Da, 1500 Da, 1000 Da, 750 DA, or 500 Da.

A compound library may be prepared or obtained by any means including, but not limited to, combinatorial chemistry techniques, fermentation methods, plant and cellular extraction procedures and the like. A library may be obtained from synthetic or from natural sources such as for example, microbial, plant, marine, viral and animal materials. Methods for making libraries are well-known in the art. [See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein.] Compound libraries may also be obtained from commercial sources (for example, from Maybridge, ChemNavigator.com, Timtec Corporation, ChemBridge Corporation, A-Syntese-Biotech ApS, Akos-SC, G & J Research Chemicals Ltd., Life Chemicals, Interchim S.A., and Spectrum Info. Ltd.).

The term “modulator” refers to a molecule or group of molecules that directly or indirectly change or alter structural, regulatory, or biochemical functions of amyloid and/or aggregates, fibrils or components thereof (e.g. monomers and oligomers), in particular Aβ, more particularly Aβ aggregates or fibrils; inhibit, reduce, reverse or disrupt aggregation, formation, deposition, accumulation, persistence or assembly of amyloid, in particular Aβ; bind to or interact with amyloid and/or aggregates, fibrils or components thereof (e.g. monomers and oligomers), in particular Aβ, more particularly Aβ aggregates or fibrils; interfere with the binding or interaction of amyloid, in particular Aβ monomers, to an Amyloid target, in particular an Aβ target; and/or breaks down the Amyloid target, in particular Aβ target. Modulators may be organic or inorganic, small to large molecular weight individual compounds, mixtures and combinatorial libraries of inhibitors, agonists, antagonists, and biopolymers such as peptides, nucleic acids, or oligonucleotides. A modulator may be a natural product or a naturally-occurring small molecule organic compound. In particular, a modulator may be a carbohydrate, monosaccharide, oligosaccharide, polysaccharide, amino acid, peptide, oligopeptide, polypeptide, protein, receptor, nucleic acids, nucleoside, nucleotide, oligonucleotide, polynucleotide, including DNA and DNA fragments, RNA and RNA fragments and the like, lipid, retinoid, steroid, glycopeptide, glycoprotein, proteoglycan and the like, and synthetic analogues or derivatives thereof, including peptidomimetics, small molecule organic compounds and the like, and mixtures thereof. A modulator identified according to the invention is preferably useful in the treatment of a disease disclosed herein (e.g. Alzheimer's disease).

The term “putative modulator” refers to a modulator whose activity, affinity or specificity for an Amyloid target, in particular an Aβ target, if any, has not been determined. Putative modulators may comprise individual compounds, mixtures of compounds, or compound libraries, preferably compound libraries.

The term “natural products” refers to compounds isolated from natural sources, such as cells, plants, fungi, animals and the like.

The term “small molecule organic compounds” refers to organic compounds generally having a molecular weight less than about 5000, 4000, 3000, 2000, 1000, 800, 600, 500, 250 or 100 Daltons, preferably less than about 500 Daltons. A small molecule organic compound may be prepared by synthetic organic techniques, such as by combinatorial chemistry techniques, or it may be a naturally-occurring small molecule organic compound.

The term “naturally-occurring small molecule organic compound(s)” refers to a natural product that is an organic compound generally having a molecular weight less than about 5000, 4000, 3000, 2000, 1000, 800, 600, 500, 250 or 100 Daltons, preferably less than about 500 Daltons.

In aspects of the invention, a modulator is a cyclohexane polyalcohol compound including derivatives thereof, in particular a cyclohexane polyalcohol compound with a scyllo- or epi-configuration.

The term “support” refers to an inert material or molecule to which an Amyloid target, in particular an Aβ target, may be immobilized, (e.g., bound or coupled, either directly or through a linking arm). Supports are well-known in the art and many are commercially available. A support may be a solid phase support including without limitation resin beads, glass beads, silica chips, capillaries, dextran, Sephadex, Sepharose, carboxymethyl cellulose polystyrene, ion-exchange resin, amino acid copolymer, or agarose. A support may be in the shape of, for example, a tube, beads, disc, sphere, column, etc. In aspects, a solid phase support in the form of a column is employed comprising from about 1 to 50 nmol, 1 to 25 nmol, 1 to 15 nmol, 1 pmol to 50 nmol, 1 pmol to 25 nmol, 1 pmol to about 15 nmol, 1 pmol to about 10 nmol, 1 pmol to 5 nmol, 5 pmol to 50 nmol, 5 pmol to 25 nmol, 5 pmol to about 15 nmol, 5 pmol to about 10 nmol, 5 pmol to 5 nmol, 10 pmol to 50 nmol, 10 pmol to 25 nmol, 10 pmol to about 15 nmol, 10 pmol to about 10 nmol, 5 pmol to 50 nmol, 1 pmol to 500 pmol, 1 pmol to 250 pmol, 1 pmol to about 150 pmol, 1 pmol to about 100 pmol, 1 pmol to 50 pmol, 10 pmol to 500 pmol, 10 pmol to 250 pmol, 10 pmol to about 150 pmol, 10 pmol to about 100 pmol, 10 pmol to 50 pmol, in particular about 1 pmol to about 10 nmol target binding or interacting sites, more particularly, from about 10 pmol to about 250 pmol target binding or interacting sites.

The term “supplemental diluent” or “make-up flow” refers to a solution or solvent which is combined with the effluent from a column before the effluent passes into a mass spectrometer, in particular an electrospray mass spectrometer. A supplemental diluent can comprise a major amount of an organic solvent and a minor amount of an aqueous buffer. Suitable organic solvents include acetonitrile, methanol and isopropanol.

“Amyloid target” refers to amyloid including without limitation amyloid β-peptide (Aβ), AA amyloid, AL amyloid, IAPP amyloid, PrP amyloid, α2-microglobulin amyloid, transthyretin, prealbumin, procalcitonin, especially Aβ amyloid and IAPP amyloid especially Aβ amyloid, and aggregates, fibrils or components thereof (e.g., monomers and oligomers). In particular aspects of the invention the Amyloid target is an Aβ target. “Aβ target” refers to Aβ oligomers, aggregated Aβ, or Aβ fibrils. In aspects of the invention the Aβ target comprises Aβ fibrils, in particular Aβ fibrils immobilized on a solid support, more particularly Aβ fibrils immobilized on a column. An Amyloid target, in particular an Aβ target, can be prepared using methods known in the art. In an aspect, Aβ fibrils are prepared by the methods of Kheterpal I. et al, Biochemistry 2001, 40(39):11757 and Cannon M J et al., Anal Biochem 2004 328(1):67, and immobilized to a solid support such as beads.

The term “total ion chromatogram” refers to a plot of ion abundance vs. time constructed from a summation of all ion intensities in a scan. In a total ion chromatogram, the number of scans is linearly related to time.

The term “void volume” or “V₀” refers to the volume of solution which passes through a frontal affinity chromatography column from the point of infusion to the point of detection. Putative modulators having no affinity for the Amyloid target, in particular an Aβ target, will typically elute from the column at the void volume.

A “void marker compound” or “void marker” includes a substance that elutes from a column at the void volume. Preferably a void marker compound does not interact with, or has no affinity for the Amyloid target. The void marker compound can be used to identify the void volume of a column used under frontal chromatography conditions. In some cases, putative modulators in a compound library which have no affinity for the target may serve as the void marker compounds. M3 is an example of a void marker compound for use in a method of the present invention.

“Electrospray” refers to the generation of gas-phase ions from a flowing solution. Electrospray is typically performed at atmospheric pressure in an electric field with or without assisted nebulization and solvent evaporation.

“Effluent” refers to a solvent or solution emerging or exiting from a frontal affinity chromatography column.

“Frontal affinity chromatography (FAC) conditions” refers to chromatography conditions in which a solution of putative modulators is applied or infused continuously at constant concentration through a column containing an immobilized Amyloid target, in particular an Aβ target, such that the target is continuously contacted with the putative modulators during the chromatography.

The term “indicator agent” refers to a compound having a known affinity or specificity for an Amyloid Target, in particular an Aβ target, and a measurable breakthrough time under frontal affinity chromatography conditions. In aspects of the invention the indicator agent is a β-amyloid monomer, in particular free β-amyloid monomer, more particularly Aβ1-42 monomers or Aβ1-40 monomers, more particularly Aβ1-42 monomers. In other aspects of the invention, the indicator agent is a cyclohexane polyalcohol compound, in particular a cyclohexane polyalcohol compound with a scyllo- or epi-configuration; more particularly a scyllo-cyclohexanehexyl compound, an epi-cyclohexanehexyl or a myo-cyclohexanehexyl compound.

The terms “interact” and “interacting” in reference to molecules refers to any physical association between molecules (e.g., putative modulators and target). The terms preferably refer to a stable association between two molecules due to, for example, electrostatic, hydrophobic, ionic and/or hydrogen-bond interactions under physiological conditions. An interaction may be either direct or indirect.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. include but are not limited to saline, buffered saline, dextrose, water, glycerol, ethanol, and combinations thereof. The use of such media and agents for an active substance is well known in the art.

A “cyclohexane polyalcohol compound” that can be employed in the invention has the base structure of the formula I:

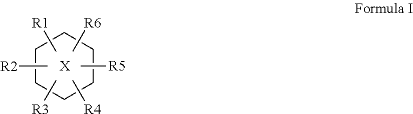

wherein X is a cyclohexane, in particular a myo-, scyllo, epi-, chiro, or allo-inositol radical wherein one or more of R¹, R², R³, R⁴, R⁵, and R⁶ are independently substituted or unsubstituted hydroxyl, alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, carboxylic ester or carboxamide, and a pharmaceutically acceptable salt, isomer, solvate, or prodrug thereof. In an aspect, R¹, R², R³, R⁴, R⁵, or R⁶ are hydroxyl. In aspects of the invention, four or five or all of R¹, R², R³, R⁴, R⁵, and/or R⁶ are hydroxyl. In particular aspects of the invention, a cyclohexanehexyl compound of the formula I is used wherein X is a radical of scyllo-inositol or epi-inositol.

Aspects of the invention use classes of compounds of the formula II:

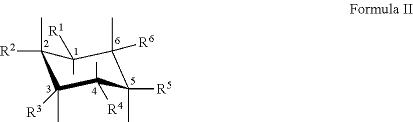

wherein R¹, R², R³, R⁴, R⁵, and R⁶ are hydroxyl, or one or more of R¹, R², R³, R⁴, R⁵, and/or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ are hydroxyl, or a pharmaceutically acceptable salt thereof.

Certain aspects of the invention use classes of compounds of the formula I or II as defined herein with the proviso that when (a) one of R¹, R², R³, R⁴, R⁵, and R⁶ are alkyl or fluorine no more than four of the other of R¹, R², R³, R⁴, R⁵, and R⁶ are hydroxyl, (b) one of R¹, R², R³, R⁴, R⁵, and R⁶ is amino or azide no more than four of R¹, R², R³, R⁴, R⁵, and R⁶ are hydroxyl, (c) two of R¹, R², R³, R⁴, R⁵, and R⁶ are amino, no more than three of R¹, R², R³, R⁴, R⁵, and R⁶ are hydroxyl, and (d) three of R¹, R², R³, R⁴, R⁵, and R⁶ are amino, carboxy, carbamyl, sulfonyl, isoxasolyl, imidazolyl, or thazolyl the other of R¹, R², R³, R⁴, R⁵, and R⁶ cannot all be hydroxyl.

In aspects of the invention, the cyclohexane polyalcohol compound is a compound of the formula III,

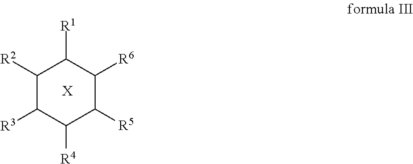

wherein X is a cyclohexane ring, where R¹, R², R³, R⁴, R⁵, and R⁶ are hydroxyl, or at least one of R¹, R², R³, R⁴, R⁵, and R⁶ is independently selected from hydrogen, C₁-C₆alkyl, C₂-C₆alkenyl, C₂-C₆ alkynyl, C₁-C₆alkoxy, C₂-C₆ alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀heterocyclic, C₁-C₆acyl, C₁-C₆acyloxy, —NH₂, —NHR⁷, —NR⁷R⁸, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀cycloalkyl, C₄-C₁₀cycloalkenyl, C₆-C₁₀aryl, C₆-C₁₀ aryl C₁-C₃alkyl, C₆-C₁₀ heteroaryl and C₃-C₁₀heterocyclic, and at least one of the remainder of R¹, R², R³, R⁴, R⁵, or R⁶ is hydroxyl; or a pharmaceutically acceptable salt thereof. In particular aspects the invention utilizes isomers of the compound of the formula III, more particularly scyllo- or epi-isomers.

In aspects of the invention, the cyclohexane polyalcohol compound is a compound of the formula IV,

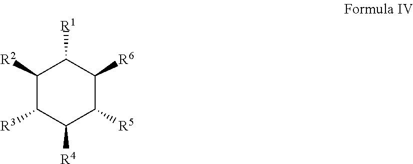

wherein R¹, R², R³, R⁴, R⁵, and R⁶ are defined as for formula III, or a pharmaceutically acceptable salt thereof.

While broad definitions of cyclohexane polyalcohol compounds are described herein for use in the present invention, certain compounds of formula I, II, III or IV may be more particularly described.

Classes of compounds that may be used in the present invention include substantially pure compounds of the formula I, II, III or IV wherein one or more of, two or more of, or three or more of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide and the other of R¹, R², R³, R⁴, R⁵, or R⁶ is a hydroxyl with the proviso that (a) when one of R¹, R², R³, R⁴, R⁵, and R⁶ are alkyl or fluorine no more than four of the other of R¹, R², R³, R⁴, R⁵, and R⁶ are hydroxyl, (b) when one of R¹, R², R³, R⁴, R⁵, and R⁶ is amino or azide no more than four of R¹, R², R³, R⁴, R⁵, and R⁶ are hydroxyl, (c) when two of R¹, R², R³, R⁴, R⁵, and R⁶ are amino, no more than three of R¹, R², R³, R⁴, R⁵, and R⁶ are hydroxyl, and (d) R¹, R², R³, R⁴, R⁵, and R⁶ independently cannot be isopropylidene.

A particular class of compounds that may be used in the present invention includes substantially pure compounds of the formula I, II, III or IV wherein one or more of, two or more of, or three or more of R¹, R², R³, R⁴, R⁵, and R⁶ are independently alkenyl, alkynyl, alkylene, alkenylene, alkoxy, alkenyloxy, cycloalkenyl, cycloalkoxy, aryl, aryloxy, arylalkoxy, aroyl, heteroaryl, heterocyclic, acyl, acyloxy, sulfonyl, sulfenyl, sulfinyl, sulfonate, sulfoxide, sulfate, nitro, cyano, isocyanato, thioaryl, thioalkoxy, seleno, silyl, silyloxy, silylthio, Cl, I, Br, carboxyl, carbonyl, carbamoyl, or carboxamide and the other of R¹, R², R³, R⁴, R⁵, or R⁶ is a hydroxyl.

In embodiments of the invention, the cyclohexane polyalcohol compound is a compound of the formula I, II, III or IV where R² is hydroxyl; and R¹, R³, R⁴, R⁵, and R⁶ are independently selected from C₁-C₆alkyl, C₂-C₆alkenyl, C₂-C₆alkynyl, C₁C₆ alkoxy, C₂-C₆alkenyloxy, C₃-C₁₀cycloalkyl, C₄-C₁₀cycloalkenyl, C₃-C₁₀cycloalkoxy, C₆-C₁₀aryl, C₆-C₁₀aryloxy, C₆-C₁₀aryl-C₁-C₃alkoxy, C₆-C₁₀aroyl, C₆-C₁₀heteroaryl, C₃-C₁₀ heterocyclic, C₁-C₆acyl, C₁-C₆acyloxy, hydroxyl, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆alkyl, C₂-C₆alkenyl, C₂-C₆alkynyl, C₃-C₁₀ cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic; provided that R¹, R², R³, R⁴, R⁵, and R⁶ are not all hydroxyl.

In embodiments of the invention, the cyclohexane polyalcohol compound is a compound of the formula I, II, III or IV where R² is hydroxyl; one of R¹, R³, R⁴, R⁵, and R⁶ is hydroxyl; and four of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀ heteroaryl and C₃-C₁₀ heterocyclic.

In embodiments of the invention, the cyclohexane polyalcohol compound is a compound of the formula I, II, III or IV where R² is hydroxyl; two of R¹, R³, R⁴, R⁵, and R⁶ are hydroxyl; and three of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexane polyalcohol compound is a compound of the formula III or IV where R² is hydroxyl; three of R¹, R³, R⁴, R⁵, and R⁶ is hydroxyl; and two of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀ cycloalkyl, C₄-C₁₀cycloalkenyl, C₃-C₁₀cycloalkoxy, C₆-C₁₀aryl, C₆-C₁₀aryloxy, C₆-C₁₀ aryl-C₁-C₃alkoxy, C₆-C₁₀aroyl, C₆-C₁₀ heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

In embodiments of the invention, the cyclohexane polyalcohol compound is a compound of the formula III or IV where R² is hydroxyl; four of R¹, R³, R⁴, R⁵, and R⁶ are hydroxyl; and one of R¹, R³, R⁴, R⁵, and R⁶ are independently selected from C₁-C₆alkyl, C₂-C₆alkenyl, C₂-C₆alkynyl, C₁C₆alkoxy, C₂-C₆alkenyloxy, C₃-C₁₀cycloalkyl, C₄-C₁₀cycloalkenyl, C₃-C₁₀cycloalkoxy, C₆-C₁₀ aryl, C₆-C₁₀aryloxy, C₆-C₁₀aryl-C₁-C₃alkoxy, C₆-C₁₀aroyl, C₆-C₁₀heteroaryl, C₃-C₁₀heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆alkenyl, C₂-C₆alkynyl, C₃-C₁₀cycloalkyl, C₄-C₁₀cycloalkenyl, C₆-C₁₀aryl, C₆-C₁₀aryl C₁-C₃alkyl, C₆-C₁₀heteroaryl and C₃-C₁₀heterocyclic.

Another particular class of compounds that may be used in the present invention includes substantially pure compounds of the formula I, II, III or IV wherein R² is hydroxyl in an equatorial position, at least one, two, three, or four of R¹, R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfenyl, sulfonyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, and the other of R¹, R³, R⁴, R⁵, and R⁶ are hydroxyl.

Another particular class of compounds that may be used in the present invention includes compounds of the formula I, II, III or IV wherein R² is hydroxyl in an equatorial position, at least two of R¹, R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, and the other of R¹, R³, R⁴, R⁵, and R⁶ are hydroxyl.

Another particular class of compounds that may be used in the present invention includes compounds of the formula I, II, III or IV wherein R² is hydroxyl in an equatorial position, at least one, two, three, or four of R¹, R², R³, R⁴, R⁵, and R⁶ are independently alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, nitro, cyano, nitro, cyano, isocyanato, Cl, Br, I, acyloxy, sulfonyl, sulfenyl, sulfinyl, sulfonate, sulfoxide, sulfate, thioalkoxy, thioaryl, carboxyl, seleno, silyl, silyloxy, silylthio, carbonyl, carbamoyl, or carboxamide, and the other of R¹, R³, R⁴, R⁵, and R⁶ are hydroxyl.

Another particular class of compounds that may be used in the present invention includes compounds of the formula I, II, III or IV wherein two of R¹, R², R³, R⁴, R⁵, and R⁶ are hydroxyl, and two or more of the other of R¹, R², R³, R⁴, R⁵, and R⁶ are independently alkyl, cycloalkyl, alkenyl, cycloalkenyl, alkynyl, alkylene, alkenylene, alkoxy, alkenyloxy, cycloalkoxy, aryl, aryloxy, arylalkoxy, aroyl, heteroaryl, heterocyclic, acyl, or acyloxy, sulfonyl, sulfenyl, sulfinyl, amino, imino, cyano, isocyanato, seleno, silyl, silyloxy, silylthio, thiol, thioalkyl, thioalkoxy, halo, carboxyl, carbonyl, carbamoyl, and carboxamide.

Another particular class of compounds that may be used in the present invention includes a compound of the formula I, II, III or IV wherein two of R¹, R², R³, R⁴, R⁵, and R⁶ are hydroxyl, and three or more of the other of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide.

Another particular class of compounds that may be used in the present invention includes a compound of the formula I, II, III or IV wherein two of R¹, R², R³, R⁴, R⁵, and R⁶ are hydroxyl, and four or more of the other of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide.

Another particular class of compounds that may be used in the present invention includes a compound of the formula I, II, III or IV wherein three of R¹, R², R³, R⁴, R⁵, and R⁶ are hydroxyl, and one or two of the other of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide.

Another particular class of compounds that may be used in the present invention includes a compound of the formula I, II, III or IV wherein three of R¹, R², R³, R⁴, R⁵, and R⁶ are hydroxyl, and two of the other of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide.

Another particular class of compounds that may be used in the present invention includes a compound of the formula I, II, III or IV wherein four of R¹, R², R³, R⁴, R⁵, and R⁶ are hydroxyl, and the other of R¹, R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfonate, sulfenyl, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide.

Another particular class of compounds that may be used in the present invention includes a compound of the formula I, II, III or IV wherein R¹, R², R⁴, R⁵, and R⁶ are hydroxyl, and R³ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide. In an embodiment, R³ is selected from the group consisting of alkenyl, alkynyl, alkylene, alkenylene, alkoxy, alkenyloxy, cycloalkyl, cycloalkenyl, cycloalkoxy, aryl, aryloxy, arylalkoxy, aroyl, imino, heteroaryl, heterocyclic, acyl, acyloxy, sulfonyl, sulfenyl, sulfinyl, sulfoxide, sulfate, thioalkoxy, thioaryl, carboxyl, carbonyl, carbamoyl, or carboxamide, in particular alkoxy, sulfonyl, sulfenyl, sulfinyl, sulfoxide, sulfate, thioalkoxy, carboxyl, carbonyl, carbamoyl, or carboxamide.

Another particular class of compounds that may be used in the present invention includes a compound of the formula I, II, III or IV wherein one, two, three, four or five of R¹, R², R³, R⁴, R⁵, and R⁶ are each independently:
(a) alkyl with 1 to 24 carbon atoms, in particular 1 to 10 or 1 to 6 carbon atoms;
(b) cycloalkyl with 3 to 16 carbon atoms, in particular 3 to 10 or 3 to 6 carbon atoms;
(c) alkenyl with 2 to 24 carbon atoms, in particular 2 to 10 or 2 to 6 carbon atoms;
(d) cycloalkenyl with 4 to 16 carbon atoms, in particular 4 to 10 or 4 to 6 carbon atoms;
(e) aryl with 4 to 24 carbon atoms, in particular 4 to 10, 4 to 8, or 4 to 6 carbon atoms;
(f) aralkyl, alkaryl, aralkenyl, or alkenylaryl;
(g) heterocyclic group comprising at least one atom selected from the group consisting of oxygen, nitrogen, and sulfur.

Another particular class of compounds that may be used in the present invention includes a compound of the formula I, II, III or IV wherein R² is hydroxyl and one, two, three, four or five of R¹, R³, R⁴, R⁵, or R⁶ are independently methyl, ethyl, propyl, butyl, pentyl, hexyl, heptyl, octyl, nonyl, decyl, dodecyl, tetradecyl, pentadecyl, hexadecyl, heptadecyl, octadecyl, eicosyl, docosyl, cyclopropyl, cyclopentyl, cyclohexyl, vinyl, allyl, propenyl, octadienyl, octenyl, decenyl, dodecenyl, tetradecenyl, hexadecenyl, octadecenyl, octadecadienyl, nonadecenyl, octadecatrienyl, arachidonyl, cyclopentenyl, cyclopentadienyl, cyclohexenyl, cyclohexadienyl, phenyl, biphenyl, terphenyl, naphtyl, anthracenyl, phenanthrenyl, pyridyl, furyl, or thiazolyl.

Another particular class of compounds that may be used in the present invention includes a compound of the formula I, II, III or IV wherein one, two, or three of R¹, R², R³, R⁴, R⁵, or R⁶ are independently —OR²⁵ where R²⁵ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide or a carbohydrate.

Another particular class of compounds that may be used in the present invention includes a compound of the formula I, II, III or IV wherein one, two or three of R¹, R², R³, R⁴, R⁵, or R⁶ are independently

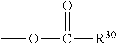

where R³⁰ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, and the other of R¹, R², R³, R⁴, R⁵, or R⁶ is hydroxyl.

Another particular class of compounds that may be used in the present invention includes a compound of the formula I, II, III or IV wherein at least one, two, three or four of R¹, R³, R⁴, R⁵, and R⁶ are hydroxyl and the other of R¹, R³, R⁴, R⁵, and R⁶ are alkyl, halo, alkoxy, sulfonyl, sulfinyl, thiol, thioalkyl, thioalkoxy, carboxyl.

Another particular class of compounds that may be used in the present invention includes a compound of the formula I, II, III or IV wherein R¹, R², R³, R⁴, R⁵, and R⁶ are independently F, N₃, NH₂, SH, NO₂, CF₃, OCF₃, SeH, Cl, Br, I or CN with the proviso that four or five of R¹, R², R³, R⁴, R⁵, and R⁶ are hydroxyl.

Another particular class of compounds that may be used in the present invention includes a compound of the formula I, II, III or IV wherein five of R¹, R², R³, R⁴, R⁵, or R⁶ are hydroxyl and one of R¹, R², R³, R⁴, R⁵, or R⁶, and more particularly R³, is selected from the group consisting of F, SeH, Cl, Br, I and CN.

Another particular class of compounds that may be used in the present invention includes a compound of the formula I, II, III or IV wherein four of R¹, R², R³, R⁴, R⁵, or R⁶ are hydroxyl and two of R¹, R², R³, R⁴, R⁵, or R⁶ are selected from the group consisting of F, —NO₂, SH, SeH, Cl, Br, I and CN.

Another particular class of compounds that may be used in the present invention includes a compound of the formula I, II, III or IV wherein four of R¹, R², R³, R⁴, R⁵, or R⁶ are hydroxyl and the other two of R¹, R², R³, R⁴, R⁵, or R⁶ are independently lower alkyl, especially methyl, ethyl, butyl, or propyl.

Another particular class of compounds that may be used in the present invention includes a compound of the formula I, II, III or IV wherein four of R¹, R², R³, R⁴, R⁵, or R⁶ are hydroxyl and the other two of R¹, R², R³, R⁴, R⁵, or R⁶ are independently lower cycloalkyl, especially cyclopropyl, cyclobutyl, and cyclopentyl.

Another particular class of compounds that may be used in the present invention includes a compound of the formula I, II, III or IV wherein one or two of R¹, R², R³, R⁴, R⁵, or R⁶ are independently carboxyl, carbamyl, sulfonyl, or a heterocyclic comprising a N atom, more particularly N-methylcarbamyl, N-propylcarbamyl, N-cyanocarbamyl, aminosulfonyl, isoxazolyl, imidazolyl, and thiazolyl.

In aspects of the invention X is a radical having a scyllo- or epi-configuration in particular X is a scyllo-inositol or epi-inositol, or configuration isomers thereof.

In embodiments of the invention, the cyclohexane polyalcohol compound is a compound of the formula I, wherein X is a radical of scyllo-inositol, epi-inositol or a configuration isomer thereof, wherein
(a) R¹, R², R³, R⁴, R⁵, and R⁶ are hydroxyl, or
(b) one or more of, two or more of, or three or more of R¹, R², R³, R⁴, R⁵, and/or R⁶ are independently optionally substituted alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carboxylic ester, carbonyl, carbamoyl, or carboxamide and the other of R¹, R², R³, R⁴, R⁵, and/or R⁶ is a hydroxyl.

In embodiments, scyllo-cyclohexanehexyl (e.g., scyllo-inositol), epi-cyclohexanehexyl (e.g., epi-inositol), myo-cyclohexanehexyl (e.g. myo-inositol), chiro-cyclohexanehexyl (e.g. chiro-inositol), or allo-cyclohexanehexyl (e.g., allo-inositol), in particular pure or substantially pure scyllo-cyclohexanehexyl or epi-cyclohexanehexyl, is employed herein.

In particular embodiments of the invention, the cyclohexane polyalcohol compound is a scyllo-cyclohexanehexyl compound, in particular pure or substantially pure scyllo-inositol. The compound “scyllo-inositol” is also referred to herein as AZD-103.

A cyclohexane polyalcohol compound includes a functional derivative of a compound of the formula I, II, III or IV. A “functional derivative” refers to a compound that possesses a biological activity (either functional or structural) that is substantially similar to the biological activity of a compound of the formula I, II, III or IV. The term “functional derivative” is intended to include “variants” “analogs” or “chemical derivatives” of a cyclohexane polyalcohol compound. The term “variant” is meant to refer to a molecule substantially similar in structure and function to a cyclohexane polyalcohol compound or a part thereof. A molecule is “substantially similar” to a cyclohexane polyalcohol compound If both molecules have substantially similar structures or if both molecules possess similar biological activity. The term “analog” refers to a molecule substantially similar in function to a cyclohexane polyalcohol compound. The term “chemical derivative” describes a molecule that contains additional chemical moieties which are not normally a part of the base molecule.

A cyclohexane polyalcohol compound of the invention includes crystalline forms of the compound which may exist as polymorphs. Solvates of the compounds formed with water or common organic solvents are also intended to be encompassed within this invention. In addition, hydrate forms of cyclohexane polyalcohol compounds and their salts, are included within this invention.

“Alkyl”, either alone or within other terms such as “thioalkyl” and “arylalkyl” means a monovalent, saturated hydrocarbon radical which may be a straight chain (i.e. linear) or a branched chain. In certain aspects of the invention, an alkyl radical comprises from about 1 to 24 or 1 to 20 carbon atoms, preferably from about 1 to 15, 1 to 10, 1 to 8, 3 to 8, 1 to 6, or 1 to 3 carbon atoms, more preferably about 1 to 6 carbon atoms. Examples of alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain embodiments of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents at positions that do not significantly interfere with the preparation of cyclohexane polyalcohol compounds and that do not significantly reduce the efficacy of the compounds. An alkyl radical may be optionally substituted with groups as defined herein. In certain aspects, an alkyl radical is substituted with one to five substituents including halo, lower alkoxy, hydroxy, cyano, nitro, thio, amino, substituted amino, carboxyl, sulfonyl, sulfenyl, sulfinyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, aryl (e.g., phenylmethyl (i.e. benzyl)), heteroaryl (e.g., pyridyl), and heterocyclic (e.g. piperidinyl, morpholinyl).

The term “alkenyl” refers to an unsaturated, acyclic branched or straight-chain hydrocarbon radical comprising at least one double bond. Alkenyl radicals may contain from about 2 to 24 or 2 to 10 carbon atoms, preferably from about 3 to 8 carbon atoms, and more preferably about 3 to 6 carbon atoms. Examples of suitable alkenyl radicals include ethenyl, propenyl such as prop-1-en-1-yl, prop-1-en-2-yl, prop-2-en-1-yl(allyl), and prop-2-en-2-yl, buten-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. Preferred alkenyl groups include ethenyl (—CH═CH₂), n-propenyl (—CH₂CH═CH₂), iso-propenyl (—C(CH₃)═CH₂), and the like. An alkenyl radical may be optionally substituted similar to alkyl. An alkenyl radical may be optionally substituted similar to alkyl.

The term “alkynyl” refers to an unsaturated, branched or straight-chain hydrocarbon radical comprising one or more triple bonds. Alkynyl radicals may contain about 1 to 20, 1 to 15, or 2 to 10 carbon atoms, preferably about 3 to 8 carbon atoms, and more preferably about 3 to 6 carbon atoms. In aspects of the invention, “alkynyl” refers to straight or branched chain hydrocarbon groups of 2 to 6 carbon atoms having one to four triple bonds. Examples of suitable alkynyl radicals include ethynyl, propynyls such as prop-1-yn-1-yl, and prop-2-yn-1-yl, butynyls such as but-1-yn-1-yl, but-1-yn-3-yl, and but-3-yn-1-yl, pentynyls such as pentyn-1-yl, pentyn-2-yl, 4-methoxypentyn-2-yl, and 3-methylbutyn-1-yl, hexynyls such as hexyn-1-yl, hexyn-2-yl, hexyn-3-yl, and 3,3-dimethylbutyn-1-yl radicals and the like. This radical may be optionally substituted similar to alkyl. The term “cycloalkynyl” refers to cyclic alkynyl groups.

The term “alkylene” refers to a linear or branched radical having from about 1 to 10, 1 to 8, 1 to 6, or 2 to 6 carbon atoms, preferably from about 1 to 5 carbon atoms, and having attachment points for two or more covalent bonds. Examples of such radicals are methylene, ethylene, ethylidene, methylethylene, and isopropylidene.

The term “alkenylene” refers to a linear or branched radical having from about 2 to 10, 2 to 8, or 2 to 6 carbon atoms, preferably from about 2 to 5 carbon atoms, at least one double bond, and having attachment points for two or more covalent bonds. Examples of such radicals are 1,1-vinylidene (CH₂═C), 1,2-vinylidene (—CH═CH—) and 1,4-butadienyl (—CH═CH—CH═CH—).

The term “halo” refers to a halogen such as fluorine, chlorine, bromine or iodine atoms, preferably fluorine or chlorine.

The term “hydroxyl” or “hydroxy” refers to a single —OH group.

The term “cyano” refers to a carbon radical having three of four covalent bonds shared by a nitrogen atom, in particular —CN.

The term “alkoxy” refers to a linear or branched oxy-containing radical having an alkyl portion of about 1 to 10 carbon atoms, such as a methoxy radical, which may be substituted. Particular alkoxy radicals are “lower alkoxy” radicals having about 1 to 6, 1 to 4 or 1 to 3 carbon atoms. An alkoxy having about 1-6 carbon atoms includes a C₁-C₆ alkyl-O-radical wherein C₁-C₆ alkyl has the meaning set out herein. Illustrative examples of alkoxy radicals include without limitation methoxy, ethoxy, propoxy, butoxy, isopropoxy and tert-butoxy alkyls. An “alkoxy” radical may optionally be further substituted with one or more substitutents disclosed herein including alkyl atoms (in particular lower alkyl) to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropoxy) and “haloalkoxyalkyl” radicals (e.g. fluoromethoxymethyl, chloromethoxyethyl, trifluoromethoxymethyl, difluoromethoxyethyl, and trifluoroethoxymethyl).

The term “alkenyloxy” refers to linear or branched oxy-containing radicals having an alkenyl portion of about 2 to 10 carbon atoms. Particular alkenyloxy radicals are “lower alkenyloxy” radicals having about 2 to 6 carbon atoms. Examples of alkenyloxy radicals include ethenyloxy, propenyloxy, butenyloxy, and isopropenyloxy alkyls. An “alkenyloxy” radical may be substituted with one or more substitutents disclosed herein including halo atoms, such as fluoro, chloro or bromo, to provide “haloalkenyloxy” radicals (e.g. trifluoroethenyloxy, fluoroethenyloxy, difluoroethenyloxy, and fluoropropenyloxy).

The term “cycloalkyl” refers to radicals having from about 3 to 15 carbon atoms and containing one, two, three, or four rings wherein such rings may be attached in a pendant manner or may be fused. In aspects of the invention, “cycloalkyl” refers to an optionally substituted, saturated hydrocarbon ring system containing 1 to 2 rings and 3 to 7 carbons per ring which may be further fused with an unsaturated C₃-C₇ carbocylic ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantyl, and the like. In certain aspects of the invention the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted with groups as disclosed herein.

The term “cycloalkenyl” refers to radicals comprising about 2 to 16, 4 to 16, 2 to 15, 2 to 10, 4 to 10, 3 to 8, 3 to 6, or 4 to 6 carbon atoms, one or more carbon-carbon double bonds, and one, two, three, or four rings wherein such rings may be attached in a pendant manner or may be fused. In certain aspects of the invention the cycloalkenyl radicals are “lower cycloalkenyl” radicals having three to seven carbon atoms, in particular cyclobutenyl, cyclopentenyl, cyclohexenyl and cycloheptenyl. A cycloalkenyl radical may be optionally substituted with groups as disclosed herein.

The term “cycloalkoxy” refers to cycloalkyl radicals attached to an oxy radical. Examples of cycloalkoxy radicals include cyclohexoxy and cyclopentoxy. A cycloalkoxy radical may be optionally substituted with groups as disclosed herein.

The term “aryl”, alone or in combination, refers to a carbocyclic aromatic system containing one, two or three rings wherein such rings may be attached together in a pendant manner or may be fused. The term “fused” means that a second ring is present (i.e, attached or formed) by having two adjacent atoms in common or shared with the first ring. In aspects of the invention an aryl radical comprises 4 to 24 carbon atoms, in particular 4 to 10, 4 to 8, or 4 to 6 carbon atoms. The term “aryl” includes without limitation aromatic radicals such as phenyl, naphthyl, indenyl, benzocyclooctenyl, benzocycloheptenyl, pentalenyl, azulenyl, tetrahydronaphthyl, indanyl, biphenyl, acephthylenyl, fluorenyl, phenalenyl, phenanthrenyl, and anthracenyl, in particular phenyl. An aryl radical may be optionally substituted with one to four substituents such as alkyl, substituted alkyl, alkenyl, substituted alkenyl, alkynyl, substituted alkynyl, aryl, substituted aryl, aralkyl, halo, trifluoromethoxy, trifluoromethyl, hydroxy, alkoxy, alkanoyl, alkanoyloxy, aryloxy, aralkyloxy, amino, alkylamino, arylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like. A substituent may be further substituted by hydroxy, halo, alkyl, alkoxy, alkenyl, alkynyl, aryl or aralkyl. In aspects of the invention an aryl radical is substituted with hydroxyl, alkyl, carbonyl, carboxyl, thiol, amino, and/or halo. The term “aralkyl” refers to an aryl or a substituted aryl group bonded directly through an alkyl group, such as benzyl. Other particular examples of substituted aryl radicals include chlorobenyzl, and amino benzyl.

The term “aryloxy” refers to aryl radicals, as defined above, attached to an oxygen atom. Exemplary aryloxy groups include napthyloxy, quinolyloxy, isoquinolizinyloxy, and the like.

The term “arylalkoxy,” as used herein, refers to an aryl group attached to an alkoxy group. Representative examples of arylalkoxy radicals include, but are not limited to, 2-phenylethoxy, 3-naphth-2-ylpropoxy, and 5-phenylpentyloxy.

The term “aroyl” refers to aryl radicals, as defined above, attached to a carbonyl radical as defined herein, including without limitation benzoyl and toluoyl. An aroyl radical may be optionally substituted with groups as disclosed herein.

The term “heteroaryl” refers to fully unsaturated heteroatom-containing ring-shaped aromatic radicals having from 3 to 15, 3 to 10, 5 to 15, 5 to 10 or 5 to 8 ring members selected from carbon, nitrogen, sulfur and oxygen, wherein at least one ring atom is a heteroatom. A heteroaryl radical may contain one, two or three rings and the rings may be attached in a pendant manner or may be fused. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyrimidyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, isoquinolyl, indazolyl, benzotriazolyl, tetrazolopyridazinyl and the like; an unsaturated 3 to 6-membered heteromonocyclic group containing an oxygen atom, in particular, 2-furyl, 3-furyl, and the like; an unsaturated 5 to 6-membered heteromonocyclic group containing a sulfur atom, in particular, 2-thienyl, 3-thienyl, and the like; unsaturated 5 to 6-membered heteromonocyclic group containing 1 to 2 oxygen atoms and 1 to 3 nitrogen atoms, in particular, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuran, benzothiophene, and the like. A heteroaryl radical may be optionally substituted with groups as disclosed herein.

The term “heterocyclic” refers to saturated and partially saturated heteroatom-containing ring-shaped radicals having from about 3 to 15, 3 to 10, 5 to 15, 5 to 10 or 3 to 8 ring members selected from carbon, nitrogen, sulfur and oxygen, wherein at least one ring atom is a heteroatom. A heterocylic radical may contain one, two or three rings wherein such rings may be attached in a pendant manner or may be fused. Examples of saturated heterocyclic radicals include without limitation a saturated 3 to 6-membered heteromonocyclic group containing 1 to 4 nitrogen atoms [e.g. pyrrolidinyl, imidazolidinyl, piperidinyl, and piperazinyl]; a saturated 3 to 6-membered heteromonocyclic group containing 1 to 2 oxygen atoms and 1 to 3 nitrogen atoms [e.g. morpholinyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyran, dihydrofuran and dihydrothiazole. Illustrative heterocyclic radicals include without limitation 2-pyrrolinyl, 3-pyrrolinyl, pyrrolidinyl, 1,3-dioxolanyl, 2H-pyranyl, 4H-pyranyl, piperidinyl, 1,4-dioxanyl, morpholinyl, 1,4-dithianyl, thiomorpholinyl, and the like.

The term “sulfate”, used alone or linked to other terms, is art recognized and includes a group that can be represented by the formula:

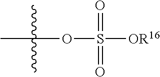

wherein R¹⁶ is an electron pair, hydrogen, alkyl, alkenyl, alkynyl, cycloalkyl, cycloalkenyl, cycloalkynyl, aryl, heterocyclic, carbohydrate, peptide or peptide derivative.

The term “sulfonyl”, used alone or linked to other terms such as alkylsulfonyl or arylsulfonyl, refers to the divalent radicals —SO₂—. In aspects of the invention where one or more of R¹, R³, R⁴, R⁵, or R⁶ is a sulfonyl group, the sulfonyl group may be attached to a substituted or unsubstituted alkyl group, alkenyl group, alkynyl group, aryl group, cycloalkyl group, cycloalkenyl group, cycloalkynyl group, heterocyclic group, carbohydrate, peptide, or peptide derivative.

The term “sulfonate” is art recognized and includes a group represented by the formula:

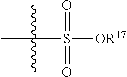

wherein R¹⁷ is an electron pair, hydrogen, alkyl, cycloalkyl, aryl, alkenyl, alkynyl, cycloalkenyl, cycloalkynyl, heterocyclic, carbohydrate, peptide, or peptide derivative.

The term “sulfinyl”, used alone or linked to other terms such as alkylsulfinyl (i.e. —S(O)— alkyl) or arylsulfinyl, refers to the divalent radicals —S(O)—.

The term “sulfoxide” refers to the radical —S═O.

The term “sulfenyl” refers to the radical SR⁹ wherein R⁹ is not hydrogen. R⁹ may be alkyl, alkenyl, alkynyl, cycloalkyl, aryl, silyl, heterocyclic, heteroaryl, carbonyl, or carboxyl.

The term “amino”, alone or in combination, refers to a radical where a nitrogen atom (N) is bonded to three substituents being any combination of hydrogen, hydroxyl, alkyl, cycloalkyl, alkenyl, alkynyl, aryl or silyl with the general chemical formula —NR¹⁰R¹¹ where R¹⁰ and R¹¹ can be any combination of hydrogen, hydroxyl, alkyl, cycloalkyl, alkenyl, alkynyl, aryl, silyl, heteroaryl or heterocyclic, which may or may not be substituted. Optionally one substituent on the nitrogen atom may be a hydroxyl group (—OH) to provide an amine known as a hydroxylamine.

Illustrative examples of amino groups are amino (—NH₂), alkylamino, acylamino, cycloamino, acycloalkylamino, arylamino, arylalkylamino, and lower alkylsilylamino, in particular methylamino, ethylamino, dimethylamino, 2-propylamino, butylamino, isobutylamino, cyclopropylamino, benzylamino, allylamino, hydroxylamino, cyclohexylamino, piperidine, benzylamino, diphenylmethylamino, tritylamino, trimethylsilylamino, and dimethyl-tert.-butylsilylamino.

The term “thiol” means —SH.

The term “thioalkyl”, alone or in combination, refers to a chemical functional group where a sulfur atom (S) is bonded to an alkyl, which may be substituted. Examples of thioalkyl groups are thiomethyl, thioethyl, and thiopropyl.

The term “thioaryl”, alone or in combination, refers to a chemical functional group where a sulfur atom (S) is bonded to an aryl group with the general chemical formula —SR¹² where R¹² is an aryl group which may be substituted. Illustrative examples of thioaryl groups and substituted thioaryl groups are thiophenyl, para-chlorothiophenyl, thiobenzyl, 4-methoxy-thiophenyl, 4-nitro-thiophenyl, and para-nitrothiobenzyl.

The term “thioalkoxy”, alone or in combination, refers to a chemical functional group where a sulfur atom (S) is bonded to an alkoxy group with the general chemical formula —SR¹³ where R¹³ is an alkoxy group which may be substituted. In aspects of the invention a “thioalkoxy” group has 1 to 6 carbon atoms and refers to a —S—(O)—C₁-C₆ alkyl group wherein C₁-C₆ lower alkyl have the meaning as defined above. Illustrative examples of a straight or branched thioalkoxy group or radical having from 1 to 6 carbon atoms, also known as a C₁-C₆ thioalkoxy, include thiomethoxy, and thioethoxy.

The term “carbonyl” refers to a carbon radical having two of the four covalent bonds shared with an oxygen atom.

The term “carboxyl”, alone or in combination, refers to —C(O)OR¹⁴— wherein R¹⁴ is hydrogen, alkyl, alkenyl, allynyl, cycloalkyl, cycloalkenyl, amino, thiol, aryl, heteroaryl, thioalkyl, thioaryl, thioalkoxy, or a heterocyclic ring, which may optionally be substituted. In aspects of the invention, the carboxyl groups are in an esterified form and may contain as an esterifying group lower alkyl groups. In particular aspects of the invention, —C(O)OR¹⁴ provides an ester or an amino acid derivative. An esterified form is also particularly referred to herein as a “carboxylic ester”. In aspects of the invention a “carboxyl” may be substituted, in particular substituted with alkyl which is optionally substituted with one or more of amino, amine, halo, alkylamino, aryl, carboxyl, or a heterocyclic. In particular aspects of the invention, the carboxyl group is methoxycarbonyl, butoxycarbonyl, tert.alkoxycarbonyl such as tert.butoxycarbonyl, arylmethyoxycarbonyl having one or two aryl radicals including without limitation phenyl optionally substituted by, for example, lower alkyl, lower alkoxy, hydroxyl, halo, and/or nitro, such as benzyloxycarbonyl, methoxybenxyloxycarbonyl, diphenylmethoxycarbonyl, 2-bromoethoxycarbonyl, 2-iodoethoxycarbonyltert.butylcarbonyl, 4-nitrobenzyloxycarbonyl, diphenylmethoxy-carbonyl, benzhydroxycarbonyl, di-(4-methoxyphenyl-methoxycarbonyl), 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine or fluorine). Examples of silicon substituents include trimethylsilyl and dimethyltert.butylsilyl.

The term “carboxamide”, alone or in combination, refers to amino, monoalkylamino, dialkylamino, monocycloalkylamino, alkylcycloalkylamino, and dicycloalkylamino radicals, attached to one of two unshared bonds in a carbonyl group.

The term “nitro” means —NO₂—.

The term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like.

The terms used herein for radicals including “alkyl”, “alkoxy”, “alkenyl”, “alkynyl”, “hydroxyl” etc. refer to both unsubstituted and substituted radicals. The term “substituted,” as used herein, means that any one or more moiety on a designated atom (e.g., hydrogen) is replaced with a selection from a group disclosed herein, provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

A radical in a cyclohexane polyalcohol compound may be substituted with one or more substituents apparent to a person skilled in the art including without limitation alkyl, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, sulfonyl, sulfenyl, alkylsulfonyl, sulfinyl, alkylsulfinyl, aralkyl, heteroaralkyl, cycloalkyl, cycloalkenyl, cycloalkoxy, cycloalkenyloxy, amino, oxy, halo, azido, thio, cyano, hydroxyl, phosphonato, phosphinato, thioalkyl, alkylamino, arylamino, arylsulfonyl, alkylcarbonyl, arylcarbonyl, heteroarylcarbonyl, heteroarylsulfinyl, heteroarylsulfony, heteroarylamino, heteroaryloxy, heteroaryloxylalkyl, arylacetamidoyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl, thioaryl, arylthioalkyl, alkoxyalkyl, and acyl groups. In embodiments of the invention, the substituents include alkyl, alkoxy, alkynyl, halo, amino, thio, oxy, and hydroxyl. Cyclohexane polyalcohol compounds can be prepared using conventional processes or they may be obtained from commercial sources. A cyclohexane polyalcohol compound can be prepared using chemical and/or microbial processes. Derivatives of cyclohexane polyalcohol compounds may be produced by introducing substituents using methods well known to a person of ordinary skill in the art.

Scyllo-cyclohexanehexyl compounds can be prepared using conventional processes or they may be obtained from commercial sources. For example, scyllo-cyclohexanehexyl compounds can be prepared using chemical and/or microbial processes. In aspects of the invention, a scyllo-inositol is produced using process steps described by M. Sarmah and Shashidhar, M., Carbohydrate Research, 2003, 338, 999-100, Husson, C., et al, Carbohyrate Research 307 (1998) 163-165; Anderson R. and E. S. Wallis, J. American Chemical Society (US), 1948, 70:2931-2935; Weissbach, A., J Org Chem (US), 1958, 23:329-330; Chung, S. K. et al., Bioorg Med. Chem. 1999, 7(11):2577-89; or Kiely D. E., and Fletcher, H. G., J. American Chemical Society (US) 1968, 90:3289-3290; described in JP09-140388, DE 3,405,663 (Merck Patent GMBH), JP04-126075, JP05-192163, or WO06109479, or described in WO0503577, US20060240534, EP1674578, JP9140388, JP09140388, JP02-184912, JP03-102492 (Hokko Chemical Industries). In particular aspects of the compositions and methods of the invention, a scyllo-inositol is prepared using the chemical process steps described in Husson, C., et al, Carbohydrate Research 307 (1998) 163-165.

In other aspects of the compositions and methods of the invention, a scyllo-inositol is prepared using microbial process steps similar to those described in WO05035774 (EP 1674578 and US20060240534) JP2003102492, or JP09140388 (Hokko Chemical Industries). Derivatives may be produced by introducing substituents into a scyllo-cyclohexanehexyl using methods well known to a person of ordinary skill in the art.

Epi-cyclohexanehexyl compounds can be prepared using conventional processes or they may be obtained from commercial sources. In aspects of the invention, an epi-inositol can be prepared using chemical and/or microbial processes. For example, an epi-inositol may be prepared by the process described by V. Pistarà(Tetrahedron Letters 41, 3253, 2000), Magasanik B., and Chargaff E. (J Biol Chem, 1948, 174:173188), U.S. Pat. No. 7,157,268, or in PCT Published Application No. WO0075355. Derivatives may be produced by introducing substituents into an epi-inositol using methods well known to a person of ordinary skill in the art.

A “disease(s)” includes a condition characterized by abnormal protein folding or aggregation or abnormal amyloid formation, deposition, accumulation or persistence, or amyloid lipid interactions. In particular aspects, the disease is a condition of the central or peripheral nervous system or systemic organ. In more particular aspects the term includes conditions associated with the formation, deposition, accumulation, or persistence of amyloid or amyloid fibrils, comprising an amyloid protein comprising or selected from the group consisting of Aβ amyloid, AA amyloid, AL amyloid, IAPP amyloid, PrP amyloid, α₂-microglobulin amyloid, transthyretin, prealbumin, and procalcitonin, especially Aβ and IAPP amyloid. A disease may be a condition where it is desirable to dissociate abnormally aggregated proteins and/or dissolve or disrupt pre-formed or pre-deposited amyloid or amyloid fibril.

In certain aspects of the invention the disease is an amyloidosis. “Amyloidosis” refers to a diverse group of diseases of acquired or hereditary origin and characterized by the accumulation of one of several different types of amyloid or amyloid fibrils. Amyloid can accumulate in a single organ or be dispersed throughout the body. The disease can cause serious problems in the affected areas, which may include the heart, brain, kidneys and digestive tract. The fibrillar composition of amyloid deposits is an identifying characteristic for various amyloid diseases. Intracerebral and cerebrovascular deposits composed primarily of fibrils of beta amyloid peptide (β-AP) are characteristic of Alzheimer's disease (both familial and sporadic forms); islet amyloid protein peptide (IAPP; amylin) is characteristic of the fibrils in pancreatic islet cell amyloid deposits associated with type II diabetes; and, β-2-microglobulin is a major component of amyloid deposits which form as a consequence of long term hemodialysis treatment. Prion-associated diseases, such as Creutzfeld-Jacob disease, scrapie, bovine spongiform encephalitis, and the like are characterized by the accumulation of a protease-resistant form of a prion protein (designated as AScr ro PrP-27).

Certain disorders are considered to be primary amyloidoses in which there is no evidence for preexisting or coexisting disease. Primary amyloidoses are typically characterized by the presence of “amyloid light chain-type” (AL-type) protein fibrils. In secondary amyloidosis there is an underlying chronic inflammatory or infectious disease state (e.g., rheumatoid arthritis, juvenile chronic arthritis, ankylosing spondylitis, psoriasis, Reiter's syndrome, Adult Still's disease, Behcet's Syndrome, Crohn's disease, chronic microbial infections such as osteomyelitis, tuberculosis, and leprosy, malignant neoplasms such as Hodgkin's lymphoma, renal carcinoma, carcinomas of the gut, lung, and urogenital tract, basel cell carcinoma, and hairy cell carcinoma). Secondary amyloidosis is characterized by deposition of AA type fibrils derived from serum amyloid A protein (ApoSSA). Heredofamilial amyloidoses may have associated neuropathic, renal, or cardiovascular deposits of the ATTR transthyretin type, and they include other syndromes having different amyloid components (e.g., familial Mediterranean fever which is characterized by AA fibrils). Other forms of amyloidosis include local forms, characterized by focal, often tumor-like deposits that occur in isolated organs. In addition, amyloidoses are associated with aging, and are commonly characterized by plaque formation in the heart or brain. Amyloidoses include systemic diseases such as adult-onset diabetes, complications from long-term hemodialysis and consequences of chronic inflammation or plasma cell dyscrasias.

A disease contemplated herein include conditions of the central or peripheral nervous system or a systemic organ that result in the deposition of proteins, protein fragments, and peptides in beta-pleated sheets, fibrils, and/or aggregates or oligomers. In particular the disease is Alzheimer's disease, presenile and senile forms; amyloid angiopathy; mild cognitive impairment; Alzheimer's disease-related dementia (e.g., vascular or Alzheimer dementia); tauopathy (e.g., argyrophilic grain dementia, corticobasal degeneration, dementia pugilistica, diffuse neurofibrillary tangles with calcification, frontotemporal dementia with parkinsonism, Hallervorden-Spatz disease, myotonic dystrophy, Niemann-Pick disease type C, non-Guamanian Motor Neuron disease with neurofibrillary tangles, Pick's disease, postencephalitic parkinsonism, cerebral amyloid angiopathy, progressive subcortical gliosis, progressive supranuclear palsy, subacute sclerosing panencephalitis, and tangle only dementia), alpha-synucleinopathy (e.g., dementia with Lewy bodies, multiple system atrophy with glial cytoplasmic inclusions, Shy-Drager syndrome, spinocerebellar ataxia (e.g., DRPLA or Machado-Joseph Disease); striatonigral degeneration, olivopontocerebellar atrophy, neurodegeneration with brain iron accumulation type I, olfactory dysfunction, and amyotrophic lateral sclerosis); Parkinson's disease (e.g., familial or non-familial); Amyotrophic Lateral Sclerosis; Spastic paraplegia (e.g., associated with defective function of chaperones and/or triple A proteins); Huntington's Disease, spinocerebellar ataxia, Freidrich's Ataxia; neurodegenerative diseases associated with intracellular and/or intraneuronal aggregates of proteins with polyglutamine, polyalanine or other repeats arising from pathological expansions of tri- or tetra-nucleotide elements within corresponding genes; cerebrovascular diseases; Down's syndrome; head trauma with post-traumatic accumulation of amyloid beta peptide; Prion related disease (Creutzfeldt-Jakob disease, Gerstmann-Straussler-Scheinker disease, and variant Creutzfeldt-Jakob disease); Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy (e.g., senile amyloid polyneuropathy or systemic Amyloidosis); Inclusion Body myositis due to amyloid beta peptide; Familial and Finnish Type Amyloidosis; Systemic amyloidosis associated with multiple myeloma; Familial Mediterranean Fever; chronic infections and inflammations; and Type II Diabetes Mellitus associated with islet amyloid polypeptide (LAPP).

In aspects of the invention, the disease is selected from the group consisting of Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, nephropathy with urticaria and deafness (Muckle-Wells syndrome), amyloidosis associated with systemic inflammatory diseases, idiopathic primary amyloidosis associated with myeloma or macroglobulinemia; amyloidosis associated with immunocyte dyscrasia; monoclonal gammopathy; occult dyscrasia; local nodular amyloidosis associated with chronic inflammatory diseases; amyloidosis associated with several immunocyte dyscrasias, familial amyloid polyneuropathy; hereditary cerebral hemorrhage with amyloidosis, Alzheimer's disease and other neurodegenerative diseases, amyloidosis associated with chronic hemodialysis, diabetes type II, insulinoma, the amyloidosis of the prion diseases, (transmissible spongiform encephalopathies prion diseases), Creutzfeldt-Jakob disease, Gerstmann-Straussler syndrome, Kuru, and scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes.

In certain aspects of the invention, the disease is a neuronal disorder (e.g., Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction and dementia).

The modulators identified using methods of the invention may also act to inhibit or prevent α-synuclein/NAC fibril formation, inhibit or prevent α-synuclein/NAC fibril growth, and/or cause disassembly, disruption, and/or disaggregation of preformed α-synuclein/NAC fibrils and α-synuclein/NAC-associated protein deposits. Examples of synuclein diseases or synucleinopathies suitable for treatment with a compound or composition of the invention are diseases associated with the formation, deposition, accumulation, or persistence of synuclein fibrils, especially α-synuclein fibrils, including without limitation Parkinson's disease, familial Parkinson's disease, Lewy body disease, the Lewy body variant of Alzheimer's disease, dementia with Lewy bodies, multiple system atrophy, olivopontocerebellar atrophy, neurodegeneration with brain iron accumulation type I, olfactory dysfunction, and the Parkinsonism-dementia complex of Guam.

In aspects of the invention, the disease is a Motor Neuron Disease associated with filaments and aggregates of neurofilaments and/or superoxide dismutase proteins, the Spastic paraplegia associated with defective function of chaperones and/or triple A proteins, or a spinocerebellar ataxia such as DRPLA or Machado-Joseph Disease.

In embodiments of the invention, the disease is Alzheimer's disease or Parkinson's disease including familial and non-familial types. In particular embodiments, the disease is Alzheimer's disease.

In certain aspects of the invention, the disease may be characterized by an inflammatory process due to the presence of macrophages by an amyloidogenic protein or peptide. A modulator identified by methods of the invention may inhibit macrophage activation and/or inhibit an inflammatory process. A modulator identified by methods of the invention may decrease, slow, ameliorate, or reverse the course or degree of macrophage invasion or inflammation in a patient.

A disease may be a condition that is associated with a molecular interaction that can be disrupted or dissociated with a modulator identified by methods of the invention. “A molecular interaction that can be disrupted or dissociated with a modulator identified by methods of the invention” includes an interaction comprising an amyloid protein and a protein or glycoprotein. An interaction comprising an amyloid protein includes an amyloid protein-amyloid protein interaction, amyloid-proteoglycan interaction, amyloid-proteoglycan/glycosaminoglycan (GAG) interaction and/or amyloid protein-glycosaminoglycan interaction. An interacting protein may be a cell surface, secreted or extracellular protein.

A disease that may be treated or prevented using a modulator identified by methods of the invention includes a disease that would benefit from the disruption or dissolution of a molecular interaction comprising an amyloid protein and an interacting compound including a protein or glycoprotein. Examples of such diseases include infectious diseases caused by bacteria, viruses, prions and fungi, including without limitation, diseases associated with pathogens including Herpes simplex virus, Pseudorabies virus, human cytomegalovirus, human immunodeficiency virus, Bordetella pertussis, Chlamydia trachomatis, Haemophilus influenzae, Helicobacter pylori, Borrelia burgdorferi, Neisseria gonorrhoeae, Mycobacterium tuberculosis, Staphylococcus aureus, Streptococcus mutans, Streptococcus suis, Plasmodium falciparum, Leishmania amazonensi, Trypanozoma cruzi, Listeria monocytogenes, Mycoplasma pneumoniae, enterotoxigenic E. coli, uropathogenic E. coli, and Pseudomonas aeruginosa.

### Methods

Methods of the invention are useful for identifying putative modulators of an amyloid, in particular Aβ. In particular the methods can be employed to analyze the affinity of members of a compound library for an Amyloid target, in particular an Aβ target, which binds or interacts with putative modulators in the library. The present invention facilitates the determination of selective modulators of any amyloid, in particular Aβ.

In aspects of the invention, mass spectrometric methods are employed for the screening of an Amyloid target, in particular an Aβ target, against compound libraries, in particular mixtures of compounds or combinatorial libraries. Prior to mass spectrometry, putative modulators may be separated using separation methods known in the art, including liquid chromatography, HPLC, CE, affinity column methods, affinity capillary electrophoresis, and size-exclusion chromatography. In particular aspects of the invention, affinity column methods are employed that select for, and separate, complexes between putative modulators and targets.

In certain methods of the invention, an Amyloid target, in particular an Aβ target, is optionally immobilized to a support. Accordingly, an Amyloid target, in particular an Aβ target, may be bound or coupled to a solid support. In aspects of the invention, an Amyloid target, in particular an Aβ target, is covalently bound or coupled to a solid support. In particular aspects of the invention porous resin beads are employed as the solid support. In other particular aspects, the solid support is porous polystyrene-divinylbenzene polymer beads, such as POROS beads (available from Perseptive Biosystems, Framingham, Mass.). In further particular aspects of the invention, the solid support comprises controlled-pore glass beads (e.g. CBX1000C beads available from Millipore.

An Amyloid target, in particular an Aβ target, can be immobilized to a support using methods known in the art. For example, an Amyloid target, in particular an Aβ target, can be bound to a support using direct immobilization techniques (e.g., covalently binding the target via a sulfhydryl, amino or carboxyl group and the like). An Amyloid target, in particular an Aβ target, may also be indirectly bound to a solid support by covalent binding through a linking or spacer arm, biotin-avidin binding, biotin-streptavidin binding, antibody binding, GST-glutathione binding, ion exchange absorption, hydrophobic interaction, fusion of the Amyloid target, in particular an Aβ target, with a peptide which binds selectively to an affinity column, and the like. Such methods are well-known in the art and kits for practicing many of these methods are commercially available. [See, for example, Stammers et al., FEBS Lett. 1991, 283, 298-302; Herman et al., Anal. Biochemistry 1986, 156, 48; Smith et al., FEBS Lett. 1987, 215, 305; Kilmartin et al., J. Cell. Biol. 1982, 93, 576-582; Skinner et al., J. Biol. Chem. 1991, 266, 14163-14166; Hopp et al., Bio/Technology 1988, 6, 1204-1210; H. M. Sassenfeld, TIBTECH 1990, 8, 88-93; Hanke et al., J. General Virology 1992, 73, 654-660; Ellison et al., J. Biol. Chem. 1991, 267, 21150-21157; U. K. Pati, Gene 1992, 114, 285-288; Wadzinski et al., J. Biol. Chem. 1992, 267, 16883-16888; Field et al., Mol. Cell. Biol. 1988, 8, 2159-2165; Gerard et al., Biochemistry 1990, 29, 9274-9281; Ausselbergs et al., Fibrinolysis 1993, 7, 1-13; Hopp et al., Biotechnology 1988, 6, 1205-1210; Blanar et al., Science 1992, 256, 1014-1018; Lin et al., J. Org. Chem. 1991, 56, 6850-6856; Zastrow et al., J. Biol. Chem. 1992, 267, 3530-3538; Goldstein et al., EMBO Jrnl. 1992, 11, 0000-0000; Lim et al., J. Infectious Disease 1990, 162, 1263-1269; Goldstein et al., Virology 1992, 190, 889-893; and the articles in IBI FLAG Epitope Vol. 1: No. 1, September 1992; and references cited therein.]

In aspects of this invention, the Amyloid target, in particular an Aβ target, is bound or coupled to a solid support using biotin-avidin, biotin-streptavidin or like binding reagents (e.g., NHS-LC-biotin available from Pierce). In this procedure, the Amyloid target, in particular an Aβ target, is typically biotinylated with a biotin reagent containing a spacer arm. The biotinylated Amyloid target, in particular an Aβ target, is then contacted with an avidin-containing solid support. The resulting biotin-avidin complex binds the Amyloid target, in particular an Aβ target, to the solid support.

Procedures for biotinylating biomolecules are well-known in the art and various biotin reagents are commercially available. See, for example, E. A. Bayer et al., Meth. Enzymol. 1990, 184, 51; U. Bickel et al., Bioconj. Chem. 1995, 6, 211; H. Hagiwara et al., J. Chromatog. 1992, 597, 331; “Avidin-Biotin Chemistry Handbook” (available from Pierce, Rockford, Ill., Catalog Item No. 15055) and references cited therein.

The extent of biotin incorporation using biotin reagents can be monitored by, for example, matrix-assisted laser desorption/ionization as described in D. C. Schriemer and L. Li, Anal. Chem. 1996, 68, 3382-3387, or by other art-recognized methods as described in the “Avidin-Biotin Chemistry Handbook” (Pierce). In particular aspects of the invention, an average of about 1 to about 50 biotins are incorporated per target molecule, more preferably about 1 to about 10 biotins per target molecule.

Avidin- or streptavidin-containing solid supports or related materials are commercially available or can be prepared by art-recognized procedures. Examples of avidin-containing supports include Ultralink-immobilized avidin (available from Pierce) and POROS 20 immobilized streptavidin (available from Perseptive Biosystems). A biotinylated Amyloid target can be coupled with an avidin-containing support by contacting the target with the support in a suitable buffer, such as phosphate buffered saline (pH 7), for about 0.5 to 4 hours at a temperature ranging from about 4° C. to about 37° C. After coupling the biotinylated target to the avidin-containing support, any remaining avidin binding sites on the support may be blocked by contacting the solid support with an excess of free biotin.

An Amyloid target, in particular an Aβ target, may be bound or coupled to a solid support either prior to or after introducing the solid support material into a column. For example, a biotinylated target may be contacted or incubated with the avidin- or streptavidin-containing solid support and the resulting solid support containing the target subsequently introduced into a column. Alternatively, the avidin- or streptavidin-containing solid support can be first introduced into the column and the biotinylated target can then be cycled through the column to form the solid support containing the target in the column. Either of these methods may also be used with any of the other previously mentioned procedures for coupling a target to a solid support.

Solid support material may be introduced into a column using conventional procedures. For example, a solid support may be slurried in a suitable diluent and the resulting slurry pressure packed or pumped into a column. Suitable diluents include, by way of example, buffers such as phosphate buffered saline (PBS) solutions, preferably containing a preservative such as sodium azide, and the like.

The nature of the Amyloid target, in particular an Aβ target, may determine the size of the column employed in this invention. Typically, the column employed in this invention will have an internal diameter (i.d.) ranging from about 1 μm to about 10 mm, 1 μm to about 5 mm, 1 μm to about 1 mm, 1 μm to about 500 μm, 1 μm to about 250 μm, 10 μm to about 10 mm, 10 μm to about 5 mm, 10 μm to about 4.6 mm, 10 μm to about 1 mm, 10 μm to about 500 μm, 10 μm to about 250 μm, 25 μm to about 10 mm, 25 μm to about 5 mm, 25 μm to about 1 mm, 25 μm to about 500 μm, 25 μm to about 250 μm, 50 μm to about 10 mm, 50 μm to about 5 mm, 50 μm to about 1 mm, 50 μm to about 500 μm, 50 μm to about 250 μm, 100 μm to about 10 mm, 100 μm to about 5 mm, 100 μm to about 1 mm, 100 μm to about 500 μm, 100 μm to about 250 μm, in particular about 10 μm to about 4.6 mm. In aspects of the invention, the internal diameter of the column will be in the range of from about 100 μm to about 250 μm. The column can typically range in length from about 1 cm to about 50 cm, 1 cm to about 40 cm, 1 cm to about 30 cm, 1 cm to about 20 cm, 2 cm to about 50 cm, 2 cm to about 40 cm, 2 cm to about 30 cm, 2 cm to about 20 cm, in particular from about 2 cm to about 20 cm. In an aspect, the column has from about 1 to 50 nmol, 1 to 25 nmol, 1 to 15 nmol, 1 pmol to 50 nmol, 1 pmol to 25 nmol, 1 pmol to about 15 nmol, 1 pmol to about 10 nmol, 1 pmol to 5 nmol, 5 pmol to 50 nmol, 5 pmol to 25 nmol, 5 pmol to about 15 nmol, 5 pmol to about 10 nmol, 5 pmol to 5 nmol, 10 pmol to 50 nmol, 10 pmol to 25 nmol, 10 pmol to about 15 nmol, 10 pmol to about 10 nmol, 5 pmol to 50 nmol, 1 pmol to 500 pmol, 1 pmol to 250 pmol, 1 pmol to about 150 pmol, 1 pmol to about 100 pmol, 1 pmol to 50 pmol, 10 pmol to 500 pmol, 10 pmol to 250 pmol, 10 pmol to about 150 pmol, 10 pmol to about 100 pmol, 10 pmol to 50 pmol, in particular about 1 pmol to about 10 nmol of Amyloid target, in particular an Aβ target, binding or interacting sites per column; more particularly, from about 10 pmol to about 250 pmol of target binding or interacting sites per column.

If an indicator agent is employed, the length of the column and its i.d. will also depend upon the K^{d} of the indicator agent (i.e., a smaller column may be used when the indicator has a higher affinity for the Amyloid target, in particular an Aβ target). Typically, when an indicator agent is employed, the column length and i.d. are selected so that the indicator agent elutes a measurable quantity after the void volume.

The body of a column employed in the invention may be comprised of any conventional column body material including, by way of illustration, poly(ether ether ketone) (PEEK), fused silica, silicon microchips, stainless steel, nylon, polyethylene, polytetrafluoroethylene (Teflon) and the like. In an aspect, the column body is comprised of poly(ether ether ketone).

After the solid support containing the target is introduced or formed in the column, the column is typically flushed with a suitable diluent to remove any unbound target or impurities. Suitable diluents for flushing the column include, for example, phosphate buffered saline, TRIS buffers and the like. If desired, a detergent may also be included in the buffer to facilitate removal of unbound target or impurities.

After the column is flushed, the column is typically equilibrated with a buffer suitable for frontal affinity chromatography and compatible with mass spectrometry. Volatile buffers are generally preferred for use with mass spectrometry. For frontal affinity chromatography, a buffer is typically selected to promote interaction of the Amyloid target, in particular an Aβ target, with putative modulators. Suitable buffers for use in FAC-MS include, by way of example, ammonium acetate, ammonium formate and the like.

Following equilibration of a column, a compound library is continuously applied to the column under frontal affinity chromatography conditions. Typically, when applied to the column, the compound library comprises a solution of the putative modulators in a suitable diluent. The diluent may be the buffer solution used to equilibrate the column. The concentration of the putative modulators in the diluent can range from about 0.01 μM to about 50 μM, 0.01 μM to about 20 μM, 0.01 μM to about 10 μM, 0.01 μM to about 5 μM, 0.1 μM to about 50 μM, 0.1 μM to about 20 μM, 0.1 μM to about 10 μM, 0.1 μM to about 5 μM, 1 μM to about 50 μM, 1 μM to about 20 μM, 1 μM to about 10 μM, or 1 μM to about 5 μM.

Procedures for conducting frontal affinity chromatography are well-known in the art. See, for example, K.-I. Kasai et al., Journal of Chromatography 1986, 376, 33-47; D. S. Hage et al., Journal of Chromatography B, 1997, 669, 449-525 and references cited therein. Generally, a compound library is continuously applied or infused into the column containing the target. Under these conditions, the target is continuously contacted or challenged with each of the putative modulators of the compound library. The column is driven to dynamic equilibrium by continuously applying the compound library to the column. Putative modulators having different binding affinities or constants to the Amyloid target, in particular an Aβ target, display different breakthrough times or hold-up volumes on the column, i.e., those members having a higher affinity for the target have a longer breakthrough time on the column or a larger hold-up volume until they begin to elute from or break through the column at their initial infusion concentration.

Frontal affinity chromatography is performed under suitable conditions known in the art. During the frontal affinity chromatography, the column is typically at a temperature in the range from about 0° C. to about 100° C., 0° C. to about 90° C., 0° C. to about 80° C., 0° C. to about 70° C., 2° C. to about 90° C., 2° C. to about 80° C., 2° C. to about 70° C., 3° C. to about 90° C., 3° C. to about 80° C., 3° C. to about 70° C., 3° C. to about 60° C., 5° C. to about 100° C., 5° C. to about 90° C., 5° C. to about 80° C., 5° C. to about 70° C., 5° C. to about 60° C., 10° C. to about 100° C., 10° C. to about 90° C., 10° C. to about 80° C., 10° C. to about 70° C., 10° C. to about 60° C., 10° C. to about 50° C., 10° C. to about 40° C., 15° C. to about 100° C., 15° C. to about 90° C., 15° C. to about 80° C., 15° C. to about 70° C., 15° C. to about 60° C., 15° C. to about 50° C., 15° C. to about 40° C., 20° C. to about 100° C., 20° C. to about 90° C., 20° C. to about 80° C., 20° C. to about 70° C., 20° C. to about 60° C., 20° C. to about 50° C., 20° C. to about 40° C., in particular from about 4° C. to about 60° C.; more particularly from about 20° C. to about 40° C.

When a putative modulator has a very high affinity for an Amyloid target, in particular an Aβ target, it may be desirable to pre-equilibrate the column with the compound library before conducting FAC-MS analysis. The column can be pre-equilibrated by either infusing the compound library through the column for a period sufficient to allow the column to reach equilibrium, (e.g., for about 0.25 to 24 hours), or by infusing the compound library into the column, stopping the flow, and allowing the system to come to equilibrium for a period of up to one day before conducting the analysis. If desired, a sequence of stop-flow cycles may also be conducted.

In aspects of methods of this invention, a mass spectrometer is coupled to the column to analyze the effluent. Mass spectrometry is particularly useful since it allows for both detection and identification of putative modulators present in the effluent. In this regard, mass spectrometry allows the eluting putative modulators of the library to be identified based on their mass/charge ratio.

Prior to analyzing the effluent from a column by mass spectrometry, the effluent is optionally diluted with a supplemental diluent or “make-up flow” and the combined flow is directed into, for example, an electrospray mass spectrometer. A supplemental diluent may comprise a major amount of an organic solvent and a minor amount of an aqueous buffer. An organic solvent may be selected so as to promote a stable and efficient electrospray. Suitable organic solvents for use in the supplemental diluent include, by way of example, acetonitrile, methanol, isopropanol and the like. In certain aspects, the organic solvent is acetonitrile. The amount of supplemental diluent employed is generally adjusted so that the combined flow rate of the effluent and the supplemental diluent is less than about 200, 150, 100, 90, 80, 70, 60, 50, 40, 30, 20 or 15 μL/min, in particular less than about 100 μL/min. In aspects of the invention, the combined flow rate entering the mass spectrometer ranges from about 100 μL/min to about 20 μL/min.

Methods for analyzing effluents using mass spectrometry are well-known in the art. Any type of mass spectrometry which is capable of directly or indirectly analyzing components present in a solution may be employed in this invention including, for example, tandem mass spectrometry (MSⁿ), collisionally activated dissociation (CAD), collisionally induced dissociation (CID), infrared multiphoton dissociation (IRMPD), atmospheric pressure chemical ionization (APCI), membrane introduction mass spectrometry (MIMS), electrospray mass spectrometry (ES-MS), continuous flow fast atom bombardment (cf-FAB), thermospray techniques, particle beam, moving belt interfaces and the like. A variety of ionization techniques may be used, including, but not limited to, electrospray, Matrix-Assisted Laser Desorption/Ionization (MALDI), and AFAB. Particular aspects of the invention employ an electrospray mass spectrometry. Apparatus and techniques for conducting electrospray mass spectrometric analysis are described, for example, in S. J. Gaskell, “Electrospray: Principles and Practice”, J. Mass. Spectrom. 1997, 32, 677-688, and references cited therein. The mass spectrometer may be of any type (i.e., scanning or dynamic) including, by way of illustration, quadrupole, time of flight, ion trap, Fourier transform ion cyclotron resonance (FT-ICR) and the like. Mass detectors that may be used in the methods of the invention include but are not limited to Fourier transform ion cyclotron resonance (FT-ICR) mass spectrometry, ion trap, quadrupole, magnetic sector, time of flight (TOF), Q-TOF, and triple quadrupole.

Mass spectrometer parameters are generally set to provide the highest sensitivity for the eluting compounds. Generally, when an electrospray mass spectrometer is employed, such adjustments will involve optimization of, for example, nebulizer pressure, drying gas flow rate, ion transmission and electrospray needle position. For example, the nebulizer pressure will typically range from about 0 psi to about 100 psi, 0 psi to about 90 psi, 0 psi to about 80 psi, 0 psi to about 70 psi or 0 psi to about 60 psi, in particular about 0 psi to about 60 psi; and the drying gas flow rate will range from about 0 L/min to about 100 L/min, about 0 L/min to about 90 L/min, about 0 L/min to about 80 L/min, about 0 L/min to about 70 L/min, about 0 L/min to about 60 L/min, or about 0 L/min to about 50 L/min, in particular about 0 L/min to about 50 L/min. A total ion chromatogram is typically measured and monitored in real-time. The size of the column, the concentration of the compound library and the flow rate will generally determine the run-time. Run times may range from about 1 min to about 80 min, about 1 min to about 70 min, about 1 min to about 60 min, about 1 min to about 50 min, in particular about 1 min to about 60 min.

Upon completion of the frontal affinity chromatography, the column may be regenerated by washing with a large volume of the binding buffer, with or without a competitive modulator. Thus, the columns may be re-used many times generally with no observable loss of activity or leaching of the Amyloid target, in particular an Aβ target.

Suitable apparatus for conducting frontal affinity chromatography are described in U.S. Pat. No. 6,054,047.

Aspects of this invention provide a method for screening a compound library to determine if any member of the library has an affinity for an Amyloid target, in particular an Aβ target, interferes with the binding or interaction of a pre-selected indicator agent or a mixture of indicator agents with the target, and/or break downs the target. In this aspect, the breakthrough time of an indicator agent having a known affinity for the target is determined after the column has been equilibrated with the compound library and compared to the breakthrough time for the indicator agent in the absence of the compound library. If the indicator agent has a shorter breakthrough time after equilibration with the compound library, the compound library contains one or more putative modulators of amyloid, for example, modulators of Aβ, having an overall affinity for the Amyloid target, in particular an Aβ target, which is higher than the indicator agent. Since an indicator agent can be selected having a relatively short breakthrough time on the column, a significant advantage of this embodiment is that compound libraries can be rapidly screened to identify those libraries having a pre-determined minimum level of activity or affinity for the Amyloid target, in particular an Aβ target. When a compound library is identified as having the pre-determined minimum level of activity or affinity for the Amyloid target, in particular an Aβ target, the library can be further analyzed using FAC-MS to identify the putative modulators interacting with or binding to the Amyloid target, in particular an Aβ target.

One advantage of using an indicator agent is that the screening time for each library is significantly reduced since only the indicator agent needs to be monitored. Additionally, in certain aspects the indicator agent binds to the Amyloid target, in particular an Aβ target, at the active site of interest, and a change in the breakthrough time for the indicator agent is only observed when a member of the library binds to, or interacts with, the same active site as the indicator agent. Accordingly, non-specific binding of the library to the Amyloid target, in particular an Aβ target, does not provide false leads.

In aspects of the invention, an indicator agent is selected that has a weak affinity for the Amyloid target, in particular an Aβ target. This permits the indicator agent to rapidly elute or breakthrough the column, thus shortening the period of time necessary to monitor the effluent. An indicator agent having a breakthrough time on the column less than about 30, 20, 15, 10, 8, 6, 5, 4, 3, 2, or 1 minutes, in particular less than about 15, 10, 5, or 1 minutes, more particularly less than about 10 or 5 minutes, in the absence of the compound library may be selected. In other aspects, an indicator having a strong affinity for the Amyloid target, in particular an Aβ target, may be used thereby allowing smaller columns to be employed. When an indicator agent having a strong affinity is used, the compound library will typically be applied to the column at a higher concentration. The breakthrough time for the indicator agent on the column in the absence of the compound library is determined using FAC-MS procedures. The affinity of the indicator agent for the Amyloid target, in particular an Aβ target, can be determined using conventional techniques, such as microcalorimetry and the like; or by using FAC-MS methods. An indicator agent may also have a unique mass in comparison to the members of the compound library so that the indicator agent can be unambiguously identified by mass spectrometry. Generally, when using an indicator agent and a quadrupole mass spectrometer, only the mass of the indicator agent is monitored to provide for better sensitivity.

Representative examples of indicator agents suitable for use with an Aβ target, include, by way of illustration, β-amyloid monomers (e.g. Aβ1-42 monomers). Additionally, more than one indicator agent may be employed. The indicator agent may also be coupled or conjugated to another molecule or contain an atom or isotope which facilitates its detection. For example, the indicator agent can be conjugated to polyethylene glycols (PEGs) so that the mass spectra would contain peaks differing by 44 units thereby facilitating detection of the of indicator agent.

When using an indicator agent, the breakthrough time for the indicator agent is first determined by applying the indicator agent to the column containing the Amyloid target, in particular an Aβ target, under frontal affinity chromatography conditions. The column is then typically equilibrated with the compound library to be screened. Generally, the compound library is applied or infused into the column for a time sufficient to allow all of the library members to breakthrough the column. In some cases, such as when very strong binding modulators are present, not all members of the library will achieve equilibrium. The effluent during this period may be presented to the mass spectrometer for analysis or may be collected for recycling or disposal. Once the column has been equilibrated (or partially equilibrated) with the compound library, a mixture comprising the compound library and the indicator agent is applied to or infused into the column using the frontal affinity chromatography procedures described herein. In an aspect, an indicator agent may be present in the mixture in an amount ranging from about 1 nM to about 10 μM, more particularly from about 1 nM to about 5 about 5 nM to about 5 μM, about 10 nM to about 5 μM, about 10 nM to about 10 μM, about 20 nM to about 5 μM, about 30 nM to about 5 μM, about 40 nM to about 5 μM, about 50 nM to about 5 μM, about 100 nM to about 5 μM, about 100 nM to about 10 μM, about 0.5 μM to about 5 μM, about 0.5 μM to about 2 μM, or about 0.5 μM to about 1 μM. The effluent from the column is analyzed to determine the breakthrough time for the indicator agent on the compound library-equilibrated column and this time period is compared to the pre-determined breakthrough time for the indicator agent to ascertain whether the compound library has a higher affinity for the Amyloid target, in particular an Aβ target, relative to the indicator agent, interferes with the interaction of the compound library and target, and/or breaks down the target.

Alternatively, the indicator agent alone can be applied or infused into the column after equilibration of the column with the compound library. This technique would allow very strongly bound modulators or those with slow off rates to be detected.

In addition to detecting the indicator agent using mass spectrometry, it is also contemplated that other methods of detection may be employed. For example, an indicator agent can be detected in the effluent from the column using, by way of example, fluorescence, infra-red absorption, UV-visible absorption, nuclear magnetic resonance (NMR), atomic spectroscopy (i.e., AAS, ICP-OES, etc.), flow cytometry and the like.

The methods of this invention allow a plurality of FAC-MS analyses to be conducted simultaneously using a single mass spectrometer to intermittently monitor each column. For example, using the methods of this invention, at least about 100 columns can be conducted simultaneously. When employing multiple columns, each column is typically monitored for a brief period of time before switching to the next column. For example, with a quadrupole mass spectrometer, each column is typically monitored sequentially for a period of about 0.5 to about 5 seconds, 0.5 seconds to about 10 seconds, about 0.5 seconds to about 20 seconds, about 0.5 seconds to about 30 seconds, about 0.5 seconds to about 40 seconds, about 1 to about 3 seconds, about 1 to about 5 seconds, about 1 to about 8 seconds, about 1 to about 10 seconds, about 1 to about 20 seconds, about 1 to about 30 seconds, about 1 to about 40 seconds, in particular for about 1 second to about 5 seconds, before switching to the next column. The effluent from each column is analyzed as described herein using mass spectrometry. Generally, a single running file is used to collect all of the data from the multiple columns thereby generating a composite total ion chromatogram. Subsequently, separate total ion chromatograms for each column are recreated by synchronizing column switching with mass spectrometry data acquisition.

In an aspect, each column has an individual electrospray needle for injection of the column's effluent into the electrospray mass spectrometer. Any geometric arrangement of multiple electrospray needles that allows for fast and repetitive sequences of needle advancement may be employed. A suitable apparatus for the injection of multiple effluents into an electrospray mass spectrometer is described in U.S. Pat. No. 6,191,418. Alternatively, a linear moving row of electrospray needles (sprayers) and the like may be employed. See, for example, Q. Xue et al., Anal. Chem. 1997, 69, 426-430 and references cited therein.

When using a plurality of columns to evaluate compound libraries using an indicator agent, each column may be run sequentially, if desired, since the run time for each of the columns is relatively short, e.g., typically about 3 minutes per column. When using an indicator agent, sequential runs of multiple columns may be advantageous since this allows the retention time for the indicator agent to be more accurately determined.

The methods of this invention also permit the determination of absolute affinity or dissociation constants, K_{d}, for selected putative modulators of a compound library. In this regard, ligands having an affinity for the Amyloid target, in particular an Aβ target, breakthrough the column at volumes (i.e., breakthrough times) related to their concentrations and K_{d} values, according to the following equation:

$${V}_{x} - {V}_{0} = \frac{{B}_{t}}{{[ X ]}_{0} + {( {K}_{d} )}_{x}}$$

where Bₜ represents the dynamic binding capacity of the column; [X]₀ is the infusion concentration of the modulator in the compound library; K^{d} is the thermodynamic dissociation constant for the modulator; V₀ is the void volume; and Vₓ represents the volume at the mid-point of the front corresponding to the breakthrough of the modulator. This simple equation indicates that, once Bₜ and the concentration of the modulator are known, the dissociation constant of a modulator can be determined from a single measurement of its Vₓ−V₀.

In order to determine Bₜ, a representative compound, e.g., compound X, is infused through the column at various concentrations and the corresponding Vₓ−V₀ values measured. A plot of ([X](V−V₀))⁻¹ versus [X]⁻¹ is generated, where the y-intercept indicates the dynamic binding capacity of the column (Bₜ) (analogous to a Lineweaver-Burk plot).

Once the dynamic binding capacity of the column has been determined, the dissociation constants for individual putative modulators of the compound library can be determined from a single FAC-MS run. For example, the K_{d} for compounds where [X]<<(K_{d})x is determined simply from Bₜ/(V−V₀). For those putative modulators with a low dissociation constant, knowledge of their concentration or infusion of the compound library at higher dilution is required to determine K₄.

The methods of the invention can further comprise determining the percentage shift in breakthrough time of an indicator agent by a putative modulator using the following equation:

% Shift=(t_{I}−t)/(t_{I}−t_{NSB})×100%

where t is the breakthrough time difference, measured at the inflection point, of the sigmoidal fronts between an indicator agent and void marker in the presence of a competing ligand(s) (i.e., modulator), t_{NSB} is the non-specific breakthrough time difference in the absence of immobilized target (and is a constant for the indicator agent used) and t_{I} is the breakthrough time difference in the absence of any competing ligands. The % Shift in the breakthrough time of an indicator compound by a putative modulator can be compared with the % Shift in the breakthrough time of an indicator agent by a compound that is known to disrupt aggregation of Aβ or Aβ oligomers such as cyclohexane polyalcohol compounds, in particular scyllo-cyclohexanehexyl (e.g, scyllo-inositol).

Therapeutic efficacy and toxicity of modulators identified using a method according to the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred. By way of example, one or more of the therapeutic effects may be demonstrated in a subject or disease model, for example, a TgCRND8 mouse with symptoms of Alzheimer's disease.

The following non-limiting example is illustrative of the present invention:

### Example

Amyloid beta (Aβ) fibrils were prepared by the methods disclosed in Kheterpal, I et al, Biochemistry, 2001 40(39):11757 and Cannon M J et al, Anal Biochem. 2004 328(1):67. The fibrils were immobilized on an affinity column and assayed by FAC-MS using the methods described in Leticia Toledo-Sherman, et al, J. Med. Chem. 2005, 48: 3221 or Slon-Usakiewicz J. J. et al, Clin. Proteom. J. 2004, 1:227-234. In particular, Aβ fibrils were immobilized to CBX1000C(COOH-modified) beads (Millipore) as follows. CBX1000C (5 mg) was activated by reaction with EDAC/NHS in 0.1M MES buffer containing 0.5 M NaCl, pH 6.4. After 45 min of mixing at room temperature the beads were centrifuged and supernatant was removed and washed with 1×MES. The beads were resuspended in 250 μL of MES buffer and 100 μg of Aβ fibrils (in 1×PBS) was added. The mixture was incubated for 2 h at room temperature and then overnight at 4° C. with 360° vertical rotation followed by incubation with 1×PBS. After loading immobilized Aβ fibrils, the FAC-MS capillary columns (250 μm id×2.5 cm) were washed with 50 μL (at 200 μL/h) of 1×PBS buffer followed by 50 μL of the running buffer (20 mM NH₄OAc containing 1% DMSO). The activity of the immobilized amyloid fibrils was determined using Aβ monomer (1 μM) as the indicator and M3 (1 μM) as the void marker in 20 mM NH₄OAc containing 1% DMSO. The makeup buffer was 90% methanol containing 0.1% acetic acid in water. Analyte solutions contained Aβ monomer (1 μM) as the indicator and M3 (1 μM) as the void marker and compounds or compound libraries ranging from 1-10 μM in 20 mM NH₄OAc containing 1% DMSO. The flow rates used were 80 μL/h for the makeup buffer and 100 μl/h for the FAC-MS columns. The column was connected to an AB/Sciex API 3000 triple-quadrupole mass spectrometer (Concord, Ontario, Canada) and syringe pumps (Harvard Biosciences, Holliston, Mass.) and was allowed to equilibrate with the running buffer until the Aβ monomer (M+H) signal was stable, then data was acquired. After 1 min, the system was switched to the analyte solution and data collection continued until the Aβ monomer signal had maximized for at least 10 min. The column was washed with running buffer until the Aβ monomer signal had reduced to its background level to regenerate the column. The data was analyzed using a customized Excel macro to determine the breakthrough times of amyloid beta and M3.

The % shift is determined from the equation:

% Shift=(t_{I}−t)/(t_{I}−t_{NSB})×100%

where t is the breakthrough time difference, measured at the inflection point, of the sigmoidal fronts between the indicator and void marker in the presence of any competing ligand(s), t_{NSB} is the non-specific breakthrough time difference in the absence of immobilized target (and is a constant for the indicator used) and t_{I} is the breakthrough time difference in the absence of any competing ligands.

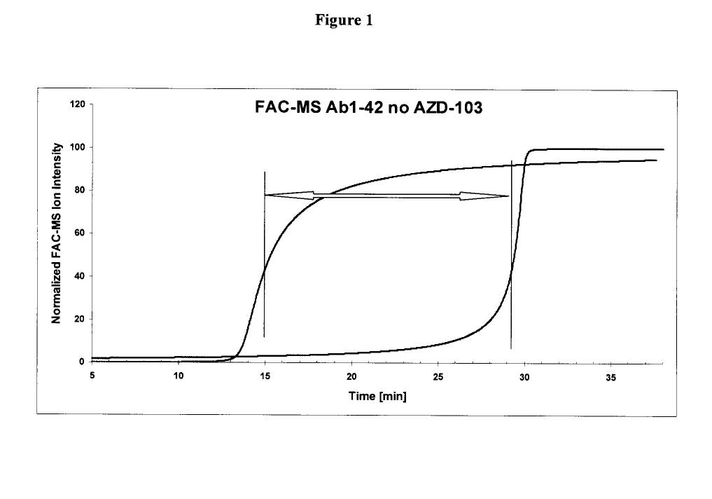

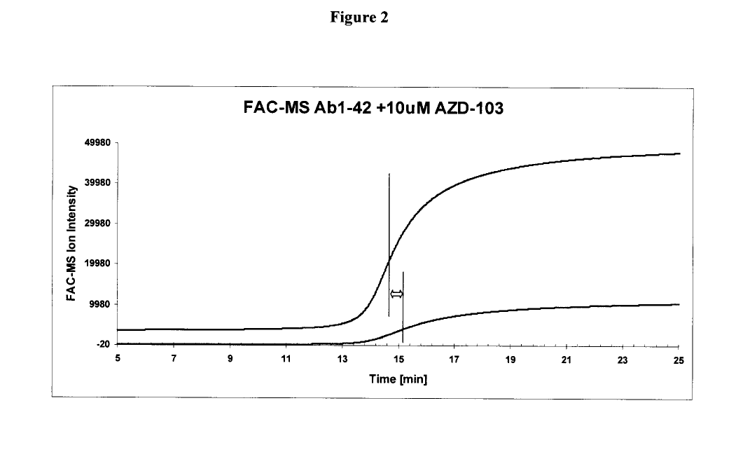

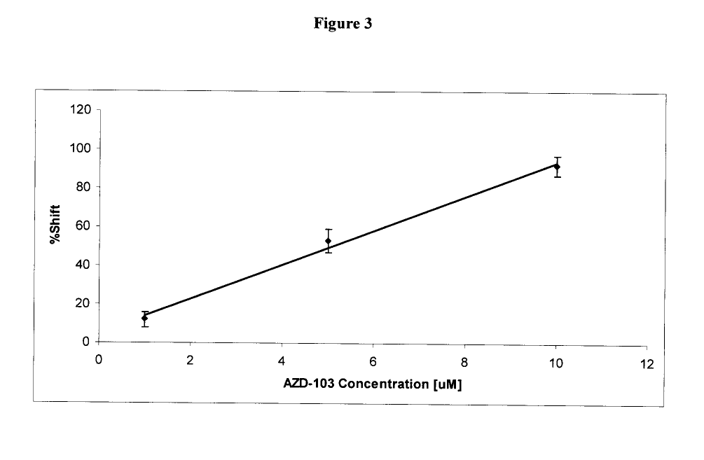

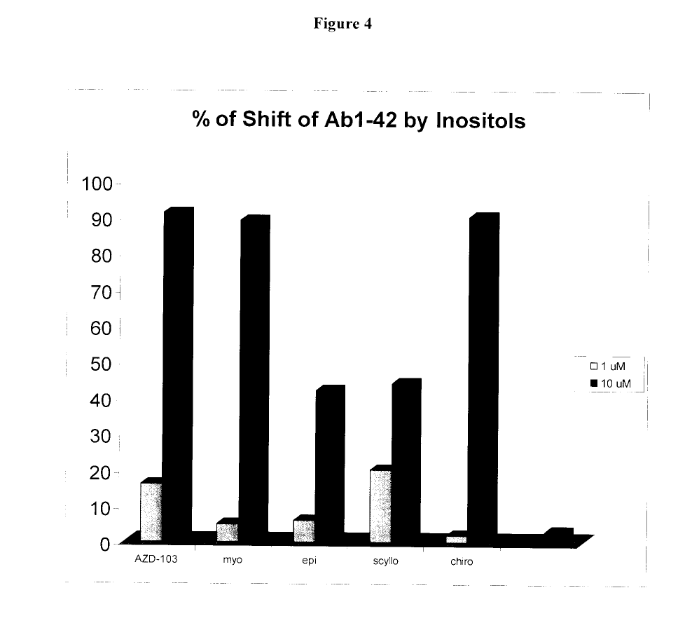

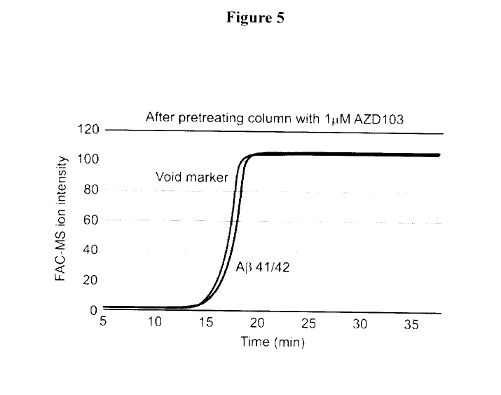

The binding time observed for the breakthrough front of free Aβ monomer was 14 minutes above the breakthrough front of the void marker, M3, indicating that binding of the Aβ monomer to the Aβ fibrils was visible by FAC-MS (FIG. 1). When 10 μM of a compound known to disrupt aggregation of Aβ or Aβ oligomers (i.e., a scyllo-cyclohexanehexyl, in particular scyllo-inositol) was added to the free Aβ monomer (1 μM) flowing through the FAC-MS column, the free Aβ monomer binding time was reduced to one minute (FIG. 2). Increasing concentrations of the scyllo-cyclohexanehexyl caused dose dependent reduction in the free Aβ monomer binding time, i.e., the breakthrough curves of the free Aβ monomer shifted to the left (greater % shift) with increasing concentrations of scyllo-cyclohexanehexyl (FIG. 3). Thus, the system was validated for screening for agents that modulate Aβ aggregation, Aβ, and/or Aβ oligomer, in particular agents that are capable of disrupting aggregation of Aβ or Aβ oligomers. Shown in FIG. 4 are the FAC-MS % shift results of the free Aβ monomer assayed with immobilized Aβ fibrils in the presence of various cyclohexanehexyls at 1 and 10 μM. FIG. 5 is a graph showing that pretreatment of immobilized Aβ aggregates with AZD-103 (1 to 10 μM) prevents binding and/or breaks down of Aβ fibrils.

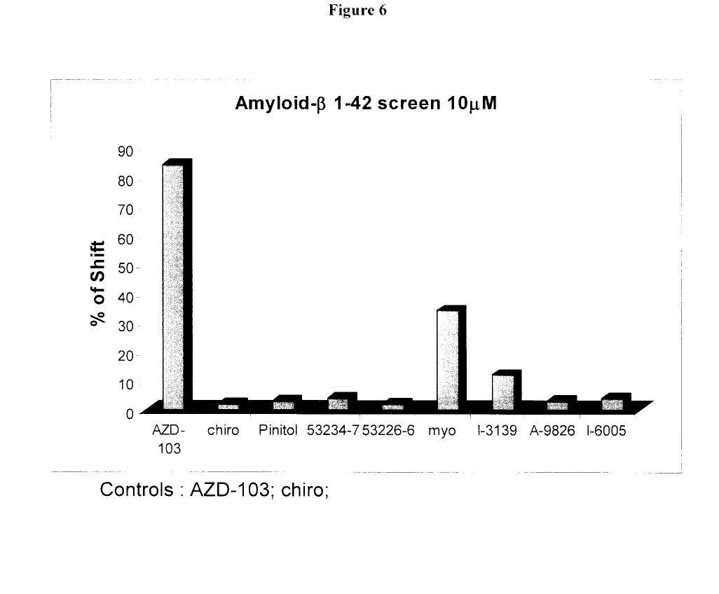

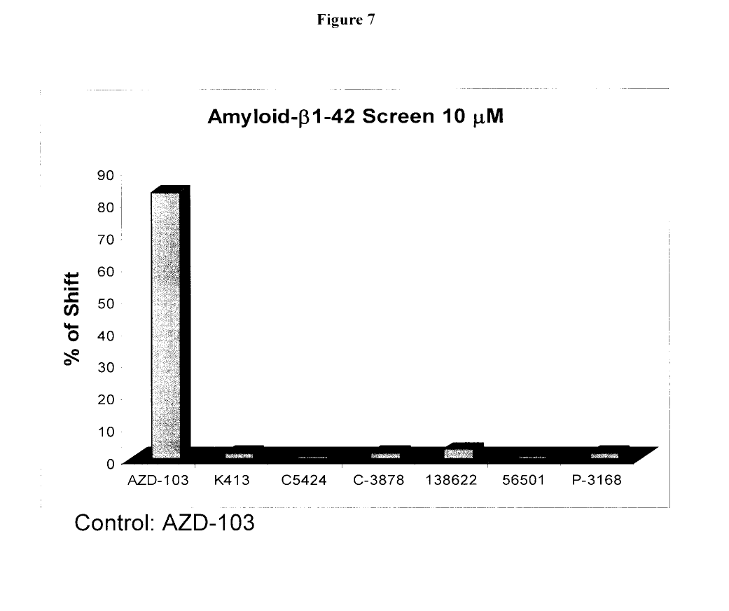

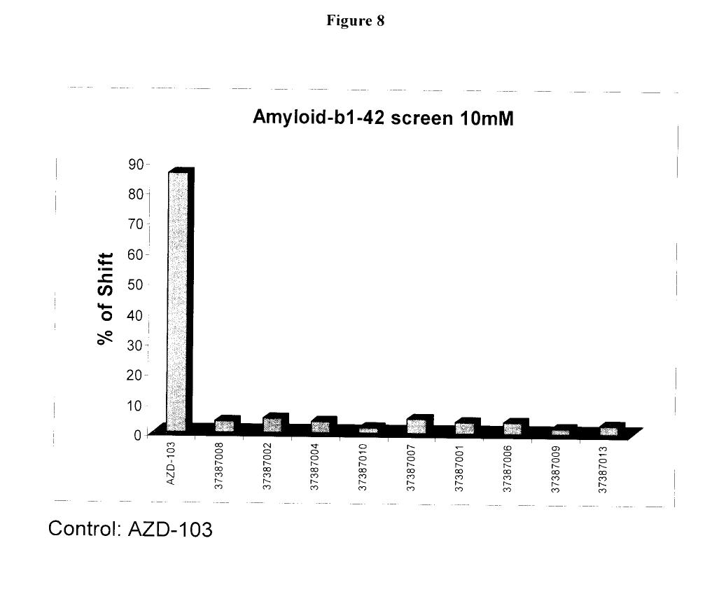

The system was used to screen for compounds that modulate Aβ aggregation. Table 1, Table 2 and FIGS. 6, 7 and 8 show the results of the screen.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the processes, methodologies etc. which are reported therein which might be used in connection with the invention. Nothing herein is to be construed as an admission that the invention is not entitled to antedate such disclosure by virtue of prior invention.

TABLE 1

| Compound | | % of |
|---|---|---|
| Identifier | Compound Name | Shift |
| AZD-103 | Scyllo-Inositol | 84 |
| Chiro | D-(+)-Chiro-Inositol | 2 |
| D-Pinitol | Methyl-D-Chiro-Inositol | 3 |
| 53234-7 | 1,2,5,6-bis-O-(1-Methylethyldene)-3-methyl-1D-chiro-inositol | 4 |
| 53226-6 | 1,2,3,4-DL-O-cyclohexyldene-5-O-methyl-L-chiro-inositol | 2 |
| Myo | Myo-Inositol | 34 |
| I-3139 | D-Myo-inositol-2,4-bis-phosphate | 12 |
| A-9826 | 2-O-b-L-Arabinopyranosyl myo-inositol | 3 |
| I-6005 | myo-inositol hexasulfate hexapotasium | 4 |
| K413 | Kasugamycin hydrochloride | 2 |
| C5424 | Conduritol B epoxide | 1 |
| C-3878 | Chlorogenic acid | 2 |
| 138622 | 1R,3R,4R,5R-Quinic acid | 3 |
| 56501 | Streptomycin sulfate | 1 |
| P-3168 | Phytic acid | 2 |
| 37387008 | 2,3,4,5,6-pentakis[(2,2-dimethylpropanoyl)oxy]cyclohexyl pivalate | 4 |
| 37387002 | 2,3,4,5-tetrakis[(2,2-dimethylpropanoyl)oxy]-6-hydroxycyclohexyl | 5 |
| | pivalate | |
| 37387004 | 2,5-bis(acetoxy)-3,4,6-tris[(2,2-dimethylpropanoyl)oxy]cyclohexyl | 4 |
| | pivalate | |
| 37387010 | 2,3,5,6-tatrakis(benzoyloxy)-4-hydroxycyclohexyl benzoate | 2 |
| 37387007 | 2,3,4,5,6-pentakis(isobutyryloxy)cyclohexyl 2-methylproanoate | 5 |
| 37387001 | 2-hydroxy-3,4,5,6-tetrakis(isobutyryloxy)cyclohexyl 2- | 4 |
| | methylpropanoate | |
| 37387006 | 2,3,4,5,6-pentakis(propionyloxy)cyclohexyl propionate | 4 |
| 37387009 | 2-hydroxy-3-4,5,6-tetrakis[(3-methylbutanoyl)oxo]cyclohexyl 3- | 2 |
| | methylbutanoate | |
| 37387013 | 2-(acetyloxy)-3,4,5,6-tetrakis[(3-methylbutanoyl)oxy]cyclohexanyl 3- | 3 |
| | methylbutanoate | |

TABLE 2

| | | | Mol | | |
|---|---|---|---|---|---|
| | Structure | Fmla | Weight | | Aβ |
| Structure | composition | Structure | Structure | Source | Shift |
| 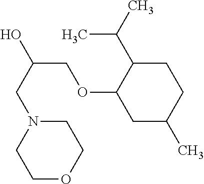 | C 68.19% H 11.11% N 4.68% O 16.03% | C17H33NO3 | 299.45746 | Asinex | 1 |
| 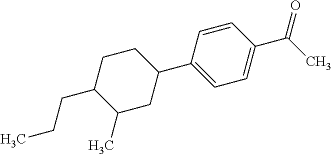 | C 83.67% H 10.14% O 6.19% | C18H26O | 258.40732 | Asinex | 1 |
| 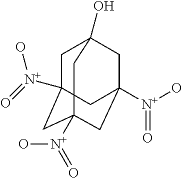 | C 41.82% H 4.56% N 14.63% O 38.99% | C10H13N3O7 | 287.23101 | Asinex | 1 |
| 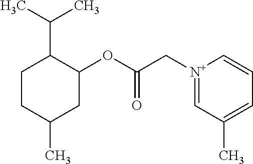 | C 74.44% H 9.72% N 4.82% O 11.02% | C18H28NO2 | 290.42936 | Asinex | 1 |
| 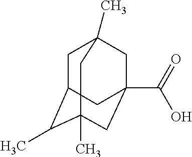 | C 75.63% H 9.97% O 14.39% | C14H22O2 | 222.33024 | Asinex | 1 |
| 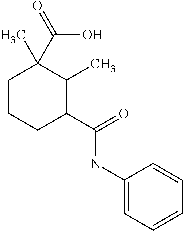 | C 69.79% H 7.69% N 5.09% O 17.43% | C16H21NO3 | 275.35067 | Asinex | 1 |
| 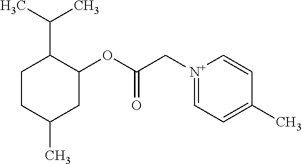 | C 74.44% H 9.72% N 4.82% O 11.02% | C18H28NO2 | 290.42936 | Asinex | 1 |
| 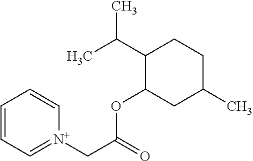 | C 73.87% H 9.48% N 5.07% O 11.58% | C17H26NO2 | 276.40227 | Asinex | 1 |
| 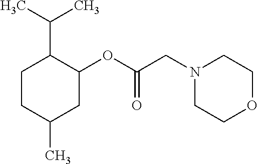 | C 67.81% H 10.31% N 4.94% O 16.94% | C16H29NO3 | 283.41443 | Asinex | 1 |
| 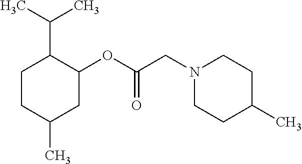 | C 73.17% H 11.26% N 4.74% O 10.83% | C18H33NO2 | 295.46921 | Asinex | 1 |
| 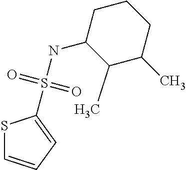 | C 52.72% H 7.00% N 5.12% O 11.70% S 23.45% | C12H19NO2S2 | 273.41873 | Asinex | 1 |
| 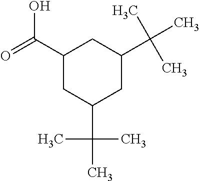 | C 74.95% H 11.74% O 13.31% | C15H28O2 | 240.38921 | Asinex | 1 |
| 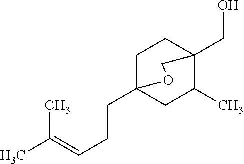 | C 75.58% H 10.99% O 13.42% | C15H26O2 | 238.37327 | Asinex | 1 |
| 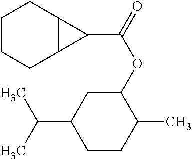 | C 77.65% H 10.86% O 11.49% | C18H30O2 | 278.4386 | Asinex | 1 |
| 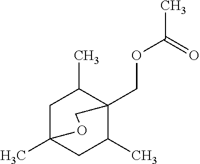 | C 68.99% H 9.80% O 21.21% | C13H22O3 | 226.31849 | Asinex | 1 |
| 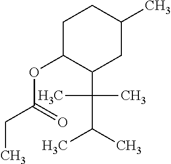 | C 75.54% H 11.89% O 12.58% | C16H30O2 | 254.4163 | Asinex | 1 |
| 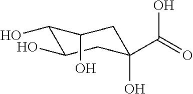 | C 43.75% H 6.29% O 49.95 | C7H12O6 | 192.17009 | Chembridge | 1 |
| 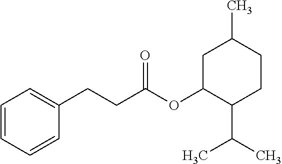 | C 79.12% H 9.79% O 11.09% | C19H28O2 | 288.43381 | Chembridge | 1 |
| 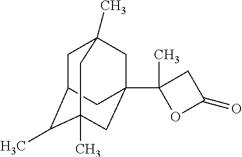 | C 77.82% H 9.99% O 12.19% | C17H26O2 | 262.39557 | Chembridge | 1 |
| 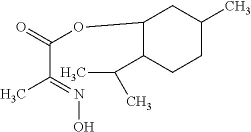 | C 64.70% H 9.61% N 5.80% O 19.89% | C13H23NO3 | 241.33316 | Chembridge | 1 |
| 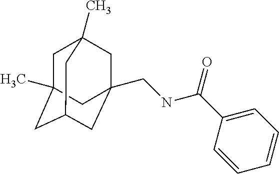 | C 80.76% H 9.15% N 4.71% O 5.38% | C20H27NO | 297.44429 | Chembridge | 1 |
| 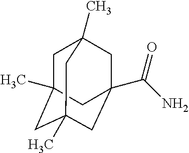 | C 75.97% H 10.47% N 6.33% O 7.23% | C14H23NO | 221.34551 | Chembridge | 1 |
| 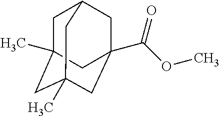 | C 75.63% H 9.97% O 14.39% | C14H22O2 | 222.33024 | Chembridge | 1 |
| 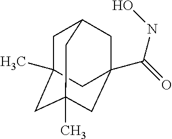 | C 69.92% H 9.48% N 6.27% O 14.33% | C13H21NO2 | 223.31782 | Chembridge | 1 |
| 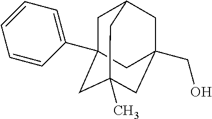 | C 84.32% H 9.44% O 6.24% | C18H24O | 256.39138 | Chembridge | 1 |
| 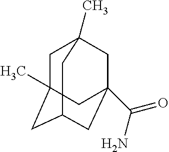 | C 75.32% H 10.21% N 6.76% O 7.72% | C13H21NO | 207.31842 | Chembridge | 1 |
| 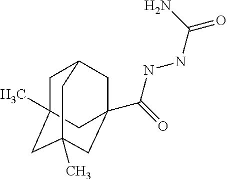 | C 63.37% H 8.74% N 15.84% O 12.06% | C14H23N3O2 | 265.35831 | Chembridge | 1 |
| 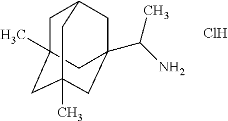 | C 68.97% H 10.75% Cl 14.54% N 5.74% | C14H26ClN | 243.82302 | Chembridge | 1 |
| 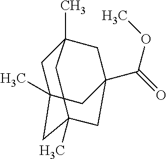 | C 76.23% H 10.24% O 13.54% | C15H24O2 | 236.35733 | Chembridge | 1 |
| 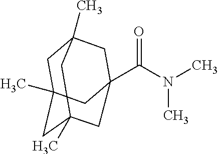 | C 77.06% H 10.91% N 5.62% O 6.42% | C16H27NO | 249.39969 | Chembridge | 1 |
| 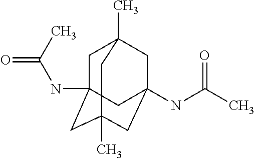 | C 69.03% H 9.41% N 10.06% O 11.49% | C16H26N2O2 | 278.39782 | Chembridge | 1 |
| 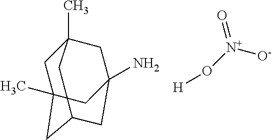 | C 59.48% H 9.15% N 11.56% O 19.81% | C12H22N2O3 | 242.32074 | Chembridge | 1 |
| 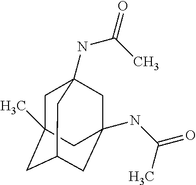 | C 68.15% H 9.15% N 10.60% O 12.10% | C15H24N2O2 | 264.37073 | Chembridge | 1 |
| 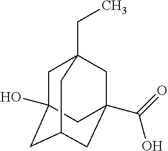 | C 69.61% H 8.99% O 21.40% | C13H20O3 | 224.30255 | Chembridge | 1 |
| 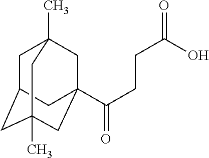 | C 72.69% H 9.15% O 18.16% | C16H24O3 | 264.36788 | Chembridge | 1 |
| 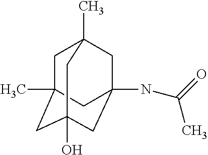 | C 70.85% H 9.77% N 5.90% O 13.48% | C14H23NO2 | 237.34491 | Chembridge | 1 |
| 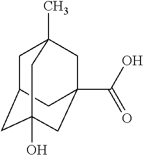 | C 68.55% H 8.63% O 22.83% | C12H18O3 | 210.27546 | Chembridge | 1 |
| 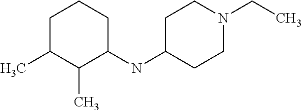 | C 75.57% H 12.68% N 11.75% | C15H30N2 | 238.41975 | Chembridge | 1 |
| 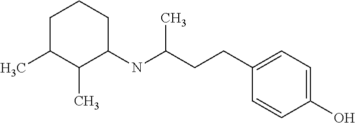 | C 78.49% H 10.61% N 5.09% O 5.81% | C18H29NO | 275.43793 | Chembridge | 1 |
| 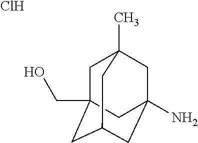 | C 62.19% H 9.57% Cl 15.30% N 6.04% O 6.90% | C12H22ClNO | 231.76824 | Chembridge | 1 |
| 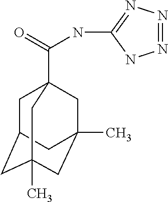 | C 61.07% H 7.69% N 25.43% O 5.81% | C14H21N5O | 275.35637 | Chembridge | 1 |
| 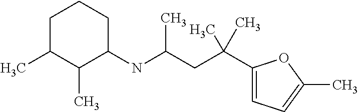 | C 78.29% H 11.41% N 4.81% O 5.49% | C19H33NO | 291.48096 | Chembridge | 1 |
| 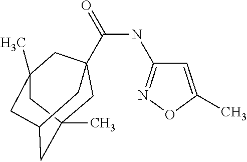 | C 70.80% H 8.39% N 9.71% O 11.10% | C17H24N2O2 | 288.39303 | Chembridge | 1 |
| 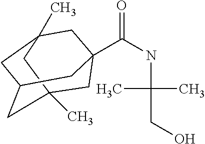 | C 73.07% H 10.46% N 5.01% O 11.45% | C17H29NO2 | 279.42618 | Chembridge | 1 |
| 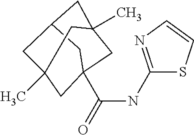 | C 66.17% H 7.64% N 9.65% O 5.51% S 11.04% | C16H22N2OS | 290.43054 | Chembridge | 1 |
| 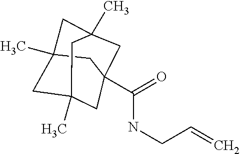 | C 78.11% H 10.41% N 5.36% O 6.12% | C17H27NO | 261.41084 | Chembridge | 1 |
| 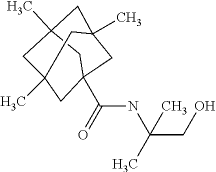 | C 73.67% H 10.65% N 4.77% O 10.90% | C18H31NO2 | 293.45327 | Chembridge | 1 |
| 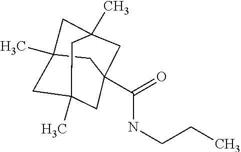 | C 77.51% H 11.10% N 5.32% O 6.07% | C17H29NO | 263.42678 | Chembridge | 1 |
| 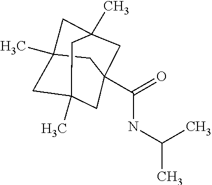 | C 77.51% H 11.10% N 5.32% O 6.07% | C17H29NO | 263.42678 | Chembridge | 1 |
| 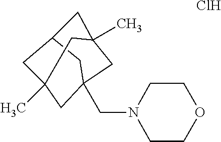 | C 68.09% H 10.08% Cl 11.82% N 4.67% O 5.34% | C17H30ClNO | 299.88775 | Chembridge | 1 |
| 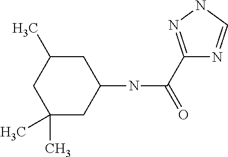 | C 60.99% H 8.53% N 23.71% O 6.77% | C12H20N4O | 236.3194 | Chembridge | 1 |
| 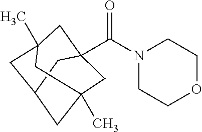 | C 73.61% H 9.81% N 5.05% O 11.53% | C17H27NO2 | 277.41024 | Chembridge | 1 |
| 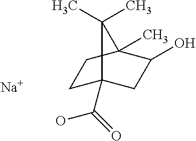 | C 59.99% H 7.78% Na 10.44% O 21.79% | C11H17NaO3 | 220.24614 | Chembridge | 1 |
| 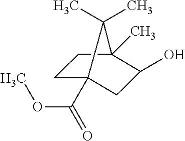 | C 67.89% H 9.50% O 22.61% | C12H20O3 | 212.2914 | Chembridge | 1 |
| 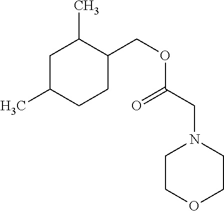 | C 66.88% H 10.10% N 5.20% O 17.82% | C15H27NO3 | 269.38734 | Chembridge | 1 |
| 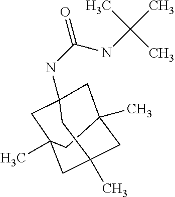 | C 73.92% H 11.03% N 9.58% O 5.47% | C18H32N2O | 292.46854 | Chembridge | 1 |
| 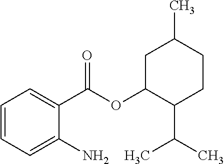 | C 74.14% H 9.15% N 5.09% O 11.62% | C17H25NO2 | 275.3943 | Chembridge | 1 |
| 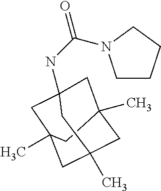 | C 74.44% H 10.41% N 9.64% O 5.51% | C18H30N2O | 290.4526 | Chembridge | 1 |
| 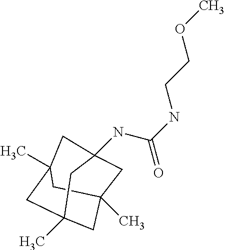 | C 69.35% H 10.27% N 9.51% O 10.87% | C17H30N2O2 | 294.44085 | Chembridge | 1 |
| 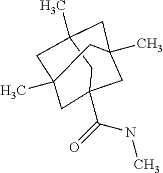 | C 76.55% H 10.71% N 5.95% O 6.80% | C15H25NO | 235.3726 | Chembridge | 1 |
| 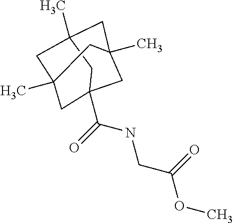 | C 69.59% H 9.28% N 4.77% O 16.36% | C17H27NO3 | 293.40964 | Chembridge | 1 |
| 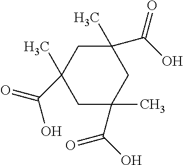 | C 55.81% H 7.03% O 37.17% | C12H18O6 | 258.27366 | Chembridge | 1 |
| 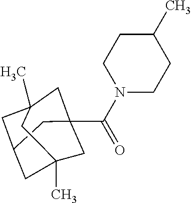 | C 78.84% H 10.80% N 4.84% O 5.53% | C19H31NO | 289.46502 | Chembridge | 1 |
| 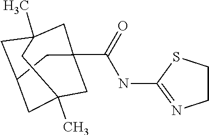 | C 65.71% H 8.27% N 9.58% O 5.47% S 10.96% | C16H24N2OS | 292.44648 | Chembridge | 1 |
| 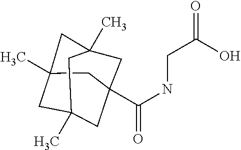 | C 68.79% H 9.02% N 5.01% O 17.18% | C16H25NO3 | 279.38255 | Chembridge | 1 |
| 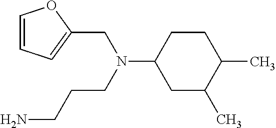 | C 72.68% H 10.67% N 10.59% O 6.05% | C16H28N2O | 264.41436 | Chembridge | 1 |
| 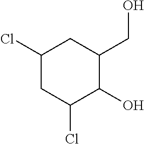 | C 42.23% H 6.08% Cl 35.62% O 16.07% | C7H12Cl2O2 | 199.07849 | Specs | 0 |
| 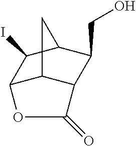 | C 36.76% H 3.77% I 43.15% O 16.32% | C9H11IO3 | 294.09062 | Specs | 0 |
| 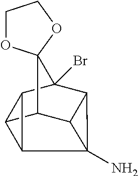 | C 48.91% H 4.48% Br 29.58% N 5.19% O 11.85% | C11H12BrNO2 | 270.12779 | Specs | 0 |
| 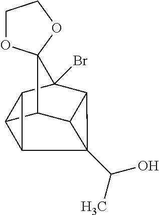 | C 52.19% H 5.05% Br 26.71% O 16.04% | C13H15BrO3 | 299.1667 | Specs | 0 |
| 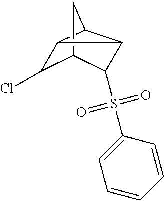 | C 58.10% H 4.88% Cl 13.19% O 11.91% S 11.93% | C13H13ClO2S | 268.76436 | Specs | 0 |
| 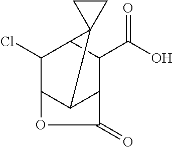 | C 54.45% H 4.57% Cl 14.61% O 26.37% | C11H11ClO4 | 242.66092 | Specs | 0 |
| 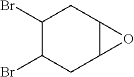 | C 28.16% H 3.15% Br 62.44% O 6.25% | C6H8Br2O | 255.93806 | Specs | 0 |
| 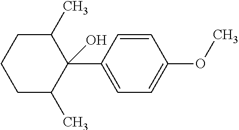 | C 76.88% H 9.46% O 13.65% | C15H22O2 | 234.34139 | Specs | 0 |
| 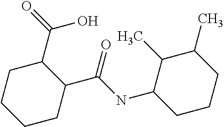 | C 68.29% H 9.67% N 4.98% O 17.06% | C16H27NO3 | 281.39849 | Specs | 0 |
| 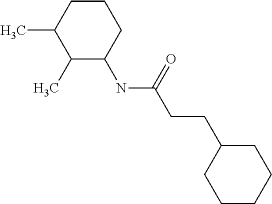 | C 76.92% H 11.77% N 5.28% O 6.03% | C17H31NO | 265.44272 | Specs | 0 |
| 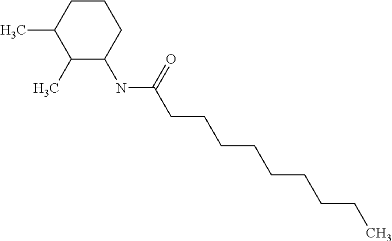 | C 76.81% H 12.53% N 4.98% O 5.68% | C18H35NO | 281.48575 | Specs | 0 |
| 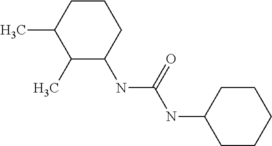 | C 71.38% H 11.18% N 11.10% O 6.34% | C15H28N2O | 252.40321 | Specs | 0 |
| 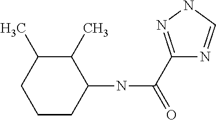 | C 59.44% H 8.16% N 25.20% O 7.20% | C11H18N4O | 222.29231 | Specs | 0 |
| 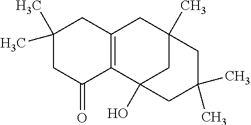 | C 78.21% H 10.21% O 11.58% | C18H28O2 | 276.42266 | Specs | 0 |
| 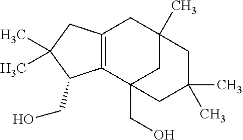 | C 78.03% H 11.03% O 10.94% | C19H32O2 | 292.46569 | Specs | 0 |
| 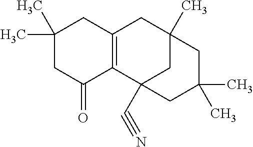 | C 79.95% H 9.53% N 4.91% O 5.61% | C19H27NO | 285.43314 | Specs | 0 |
| 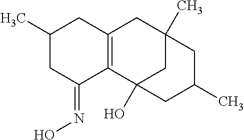 | C 72.97% H 9.57% N 5.32% O 12.15% | C16H25NO2 | 263.38315 | Specs | 0 |
| 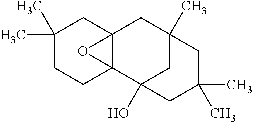 | C 77.65% H 10.86% O 11.49% | C18H30O2 | 278.4386 | Specs | 0 |
| 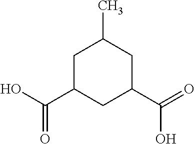 | C 58.05% H 7.58% O 34.37% | C9H14O4 | 186.20953 | Specs | 0 |
| 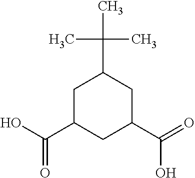 | C 63.14% H 8.83% O 28.03% | C12H20O4 | 228.2908 | Specs | 0 |
| 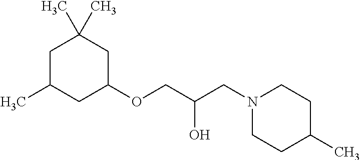 | C 72.68% H 11.86% N 4.71% O 10.76% | C18H35NO2 | 297.48515 | Specs | 0 |
| 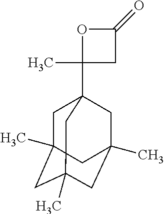 | C 77.82% H 9.99% O 12.19% | C17H26O2 | 262.39557 | Specs | 0 |
| 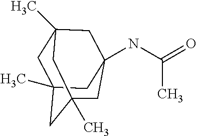 | C 76.55% H 10.71% N 5.95% O 6.80% | C15H25NO | 235.3726 | Specs | 0 |
| 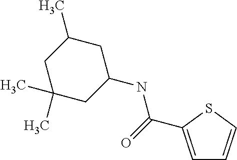 | C 66.89% H 8.42% N 5.57% O 6.36% S 12.75% | C14H21NOS | 251.39357 | Specs | 0 |
| 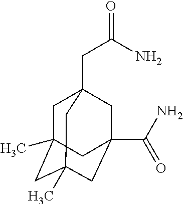 | C 68.15% H 9.15% N 10.60% O 12.10% | C15H24N2O2 | 264.37073 | Specs | 0 |
| 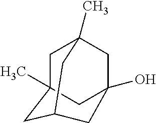 | C 79.94% H 11.18% O 8.87% | C12H20O | 180.2926 | Specs | 0 |
| 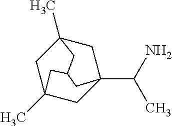 | C 81.09% H 12.15% N 6.75% | C14H25N | 207.36205 | Specs | 0 |
| 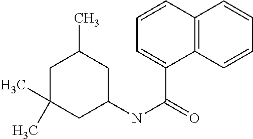 | C 81.31% H 8.53% N 4.74% O 5.42% | C20H25NO | 295.42835 | Specs | 0 |
| 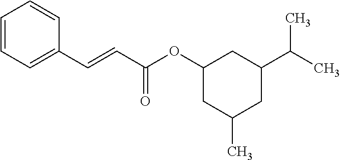 | C 79.68% H 9.15% O 11.17% | C19H26O2 | 286.41787 | Specs | 0 |
| 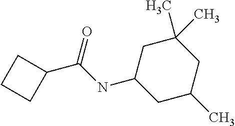 | C 75.28% H 11.28% N 6.27% O 7.16% | C14H25NO | 223.36145 | Specs | 0 |
| 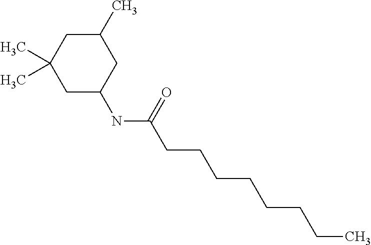 | C 76.81% H 12.53% N 4.98% O 5.68% | C18H35NO | 281.48575 | Specs | 0 |
| 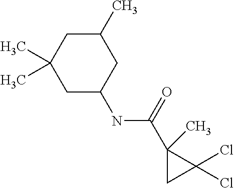 | C 57.54% H 7.93% Cl 24.26% N 4.79% O 5.47% | C14H23Cl2NO | 292.25151 | Specs | 0 |
| 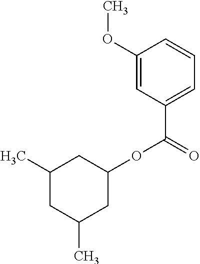 | C 73.25% H 8.45% O 18.30% | C16H22O3 | 262.35194 | Specs | 0 |
| 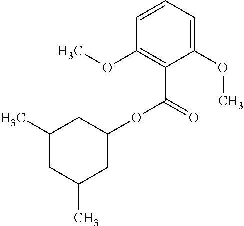 | C 69.84% H 8.27% O 21.89% | C17H24O4 | 292.37843 | Specs | 0 |
| 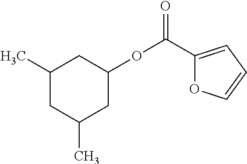 | C 70.24% H 8.16% O 21.59% | C13H18O3 | 222.28661 | Specs | 0 |
| 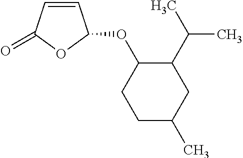 | C 70.56% H 9.30% O 20.14% | C14H22O3 | 238.32964 | Specs | 0 |
| 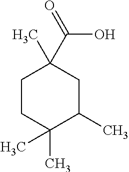 | C 71.70% H 10.94% O 17.36% | C11H20O2 | 184.28085 | Specs | 0 |
| 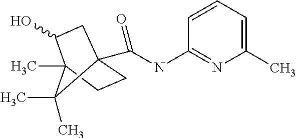 | C 70.80% H 8.39% N 9.71% O 11.10% | C17H24N2O2 | 288.39303 | Specs | 0 |
| 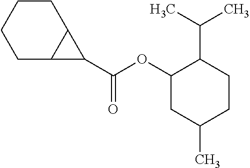 | C 77.65% H 10.86% O 11.49% | C18H30O2 | 278.4386 | Specs | 0 |
| 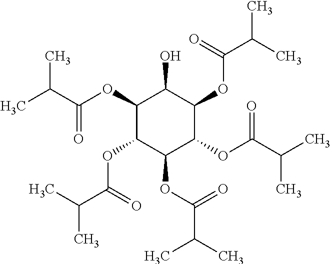 | C 58.85% H 7.98% O 33.17% | C26H42O11 | 530.61804 | Specs | 4 |
| 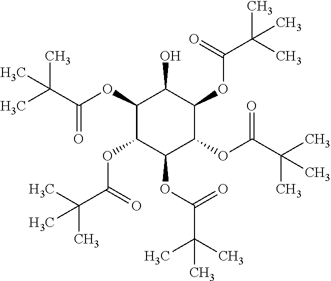 | C 61.98% H 8.73% O 29.30% | C31H52O11 | 600.75349 | Specs | 5 |
| 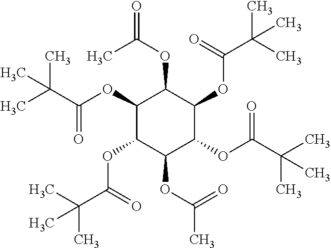 | C 59.98% H 8.05% O 31.96% | C30H48O12 | 600.70986 | Specs | 4 |
| 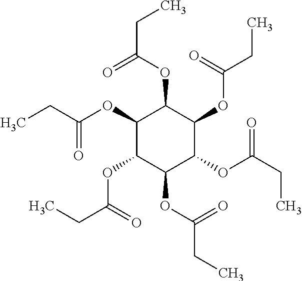 | C 55.81% H 7.03% O 37.17% | C24H36O12 | 516.54732 | Specs | 4 |
| 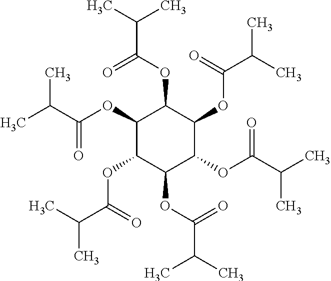 | C 59.98% H 8.05% O 31.96% | C30H48O12 | 600.70986 | Specs | 5 |
| 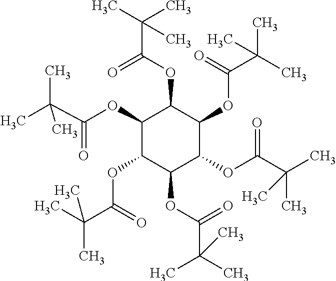 | C 63.14% H 8.83% O 28.03% | C36H60O12 | 684.8724 | Specs | 4 |
| 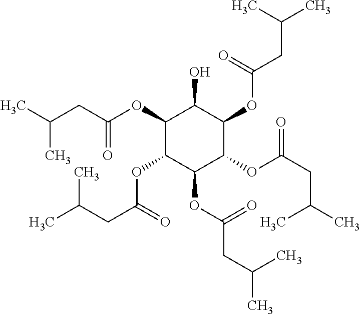 | C 61.98% H 8.73% O 29.30% | C31H52O11 | 600.75349 | Specs | 2 |
| 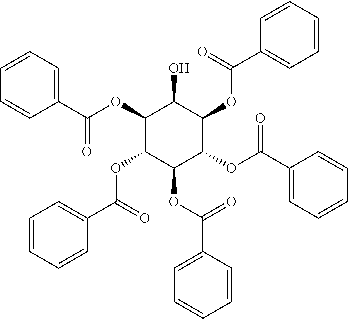 | C 70.28% H 4.60% O 25.12% | C41H32O11 | 700.70559 | Specs | 2 |
| 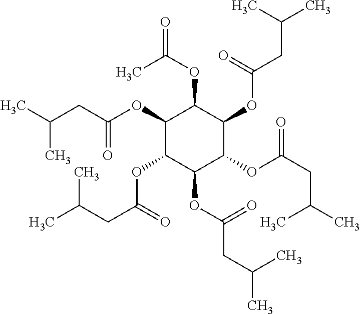 | C 61.66% H 8.47% O 29.87% | C33H54O12 | 642.79113 | Specs | 3 |
| 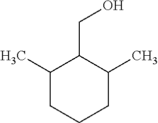 | C 76.00% H 12.76% O 11.25% | C9H18O | 142.24321 | ASDI | 1 |
| 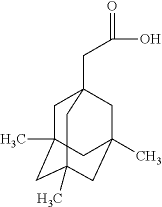 | C 76.23% H 10.24% O 13.54% | C15H24O2 | 236.35733 | ASDI_PRIM | 1 |
| 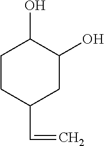 | C 67.57% H 9.92% O 22.50% | C8H14O2 | 142.19958 | ASDI | 1 |
| 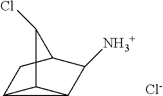 | C 46.69% H 6.16% Cl 39.38% N 7.78% | C7H11Cl2N | 180.07842 | ASDI | 1 |
| 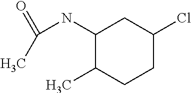 | C 56.99% H 8.50% Cl 18.69% N 7.38% O 8.43% | C9H16ClNO | 189.68697 | ASDI | 1 |
| 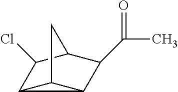 | C 63.35% H 6.50% Cl 20.78% O 9.38% | C9H11ClO | 170.64042 | ASDI | 1 |
| 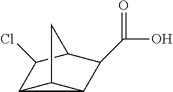 | C 55.67% H 5.26% Cl 20.54% O 18.54% | C8H9ClO2 | 172.61273 | ASDI | 1 |
| 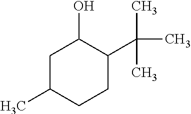 | C 77.58% H 13.02% O 9.39% | C11H22O | 170.29739 | ASDI | 1 |
| 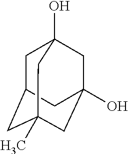 | C 72.49% H 9.95% O 17.56% | C11H18O2 | 182.26491 | ASDI_PRIM | 1 |
| 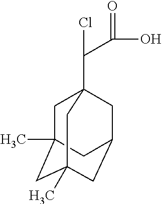 | C 65.49% H 8.24% Cl 13.81% O 12.46% | C14H21ClO2 | 256.77527 | ASDI_PRIM | 1 |
| 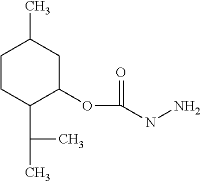 | C 61.65% H 10.35% N 13.07% O 14.93% | C11H22N2O2 | 214.31019 | ASDI | 1 |
| 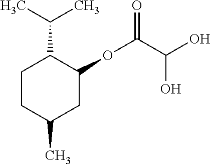 | C 62.58% H 9.63% O 27.79% | C12H22O4 | 230.30674 | ASDI | 1 |
| 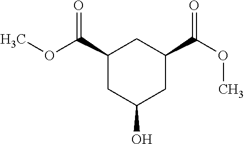 | C 55.55% H 7.46% O 37.00% | C10H16O5 | 216.23602 | ASDI_PRIM | 1 |
| 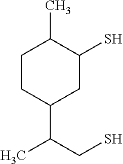 | C 58.76% H 9.86% S 31.37% | C10H20S2 | 204.3989 | ASDI | 1 |
| 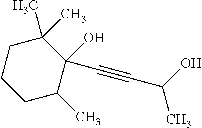 | C 74.24% H 10.54% O 15.21% | C13H22O2 | 210.31909 | ASDI | 1 |
| 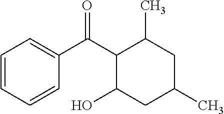 | C 77.55% H 8.68% O 13.77% | C15H20O2 | 232.32545 | ASDI | 1 |
| 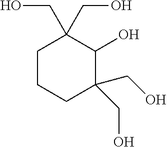 | C 54.53% H 9.15% O 36.32% | C10H20O5 | 220.2679 | ASDI | 1 |
| 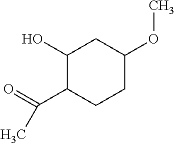 | C 62.77% H 9.36% O 27.87% | C9H16O3 | 172.22607 | ASDI | 1 |
| 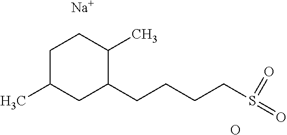 | C 53.31% H 8.57% Na 8.50% O 17.75% S 11.86% | C12H23NaO3S | 270.36911 | ASDI | 1 |
| 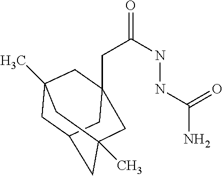 | C 64.49% H 9.02% N 15.04% O 11.45% | C15H25N3O2 | 279.3854 | ChemDiv | 2 |
| 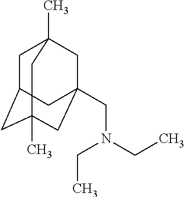 | C 81.86% H 12.53% N 5.62% | C17H31N | 249.44332 | ChemDiv | 2 |
| 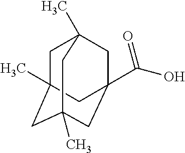 | C 75.63% H 9.97% O 14.39% | C14H22O2 | 222.33024 | ChemDiv | 2 |
| 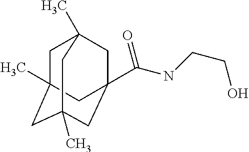 | C 72.41% H 10.25% N 5.28% O 12.06% | C16H27NO2 | 265.39909 | ChemDiv | 2 |
| 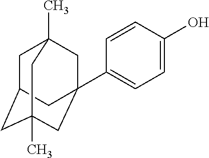 | C 84.32% H 9.44% O 6.24% | C18H24O | 256.39138 | ChemDiv | 2 |
| 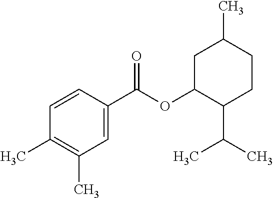 | C 79.12% H 9.79% O 11.09% | C19H28O2 | 288.43381 | ChemDiv | 2 |
| 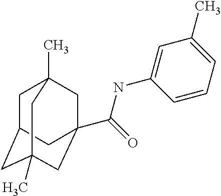 | C 80.76% H 9.15% N 4.71% O 5.38% | C20H27NO | 297.44429 | ChemDiv | 2 |
| 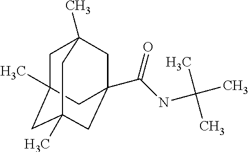 | C 77.92% H 11.26% N 5.05% O 5.77% | C18H31NO | 277.45387 | ChemDiv | 2 |
| 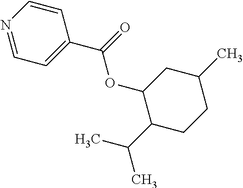 | C 73.53% H 8.87% N 5.36% O 12.24% | C16H23NO2 | 261.36721 | ChemDiv | 1 |
| 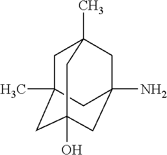 | C 73.80% H 10.84% N 7.17% O 8.19% | C12H21NO | 195.30727 | ChemDiv | 1 |
| 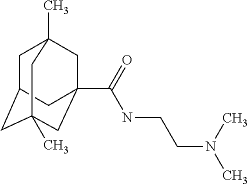 | C 73.33% H 10.86% N 10.06% O 5.75% | C17H30N2O | 278.44145 | ChemDiv | 1 |
| 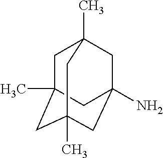 | C 80.76% H 11.99% N 7.24% | C13H23N | 193.33496 | ChemDiv | 1 |
| 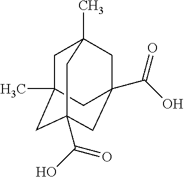 | C 66.65% H 7.99% O 25.36% | C14H20O4 | 252.3131 | ChemDiv | 1 |
| 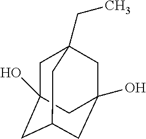 | C 73.43% H 10.27% O 16.30% | C12H20O2 | 196.292 | ChemDiv | 1 |
| 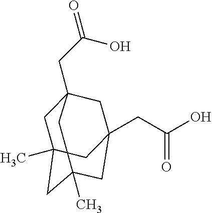 | C 68.55% H 8.63% O 22.83% | C16H24O4 | 280.36728 | ChemDiv | 1 |
| 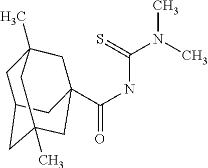 | C 65.26% H 8.90% N 9.51% O 5.43% S 10.89% | C16H26N2OS | 294.46242 | ChemDiv | 1 |
| 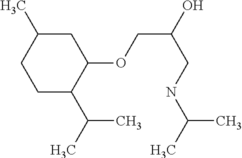 | C 70.80% H 12.25% N 5.16% O 11.79% | C16H33NO2 | 271.44691 | ChemDiv | 1 |
| 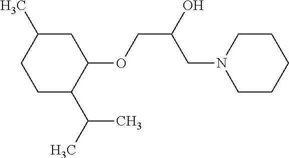 | C 72.68% H 11.86% N 4.71% O 10.76% | C18H35NO2 | 297.48515 | ChemDiv | 1 |
| 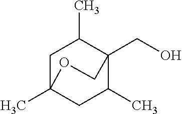 | C 71.70% H 10.94% O 17.36% | C11H20O2 | 184.28085 | ChemDiv | 1 |
| 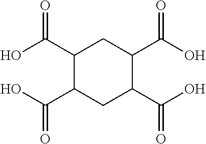 | C 46.16% H 4.65% O 49.19% | C10H12O8 | 260.20234 | ChemDiv | 1 |
| 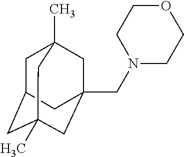 | C 77.51% H 11.10% N 5.32% O 6.07% | C17H29NO | 263.42678 | ChemDiv | 1 |
| 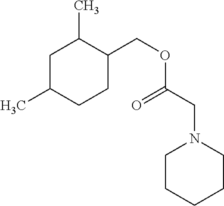 | C 71.87% H 10.93% N 5.24% O 11.97% | C16H29NO2 | 267.41503 | ChemDiv | 1 |
| 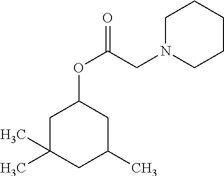 | C 71.87% H 10.93% N 5.24% O 11.97% | C16H29NO2 | 267.41503 | ChemDiv | 1 |
| 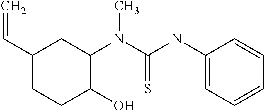 | C 66.17% H 7.64% N 9.65% O 5.51% S 11.04% | C16H22N2OS | 290.43054 | ChemDiv | 1 |
| 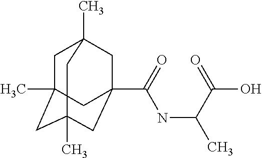 | C 69.59% H 9.28% N 4.77% O 16.36% | C17H27NO3 | 293.40964 | ChemDiv | 2 |
| 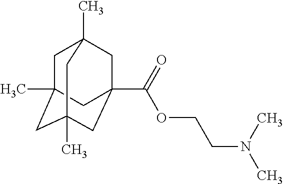 | C 73.67% H 10.65% N 4.77% O 10.90% | C18H31NO2 | 293.45327 | ChemDiv | 2 |
| 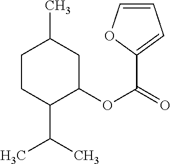 | C 71.97% H 8.86% O 19.17% | C15H22O3 | 250.34079 | ChemDiv | 2 |
| 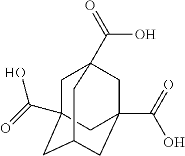 | C 58.20% H 6.01% O 35.78% | C13H16O6 | 268.26887 | ChemDiv | 2 |
| 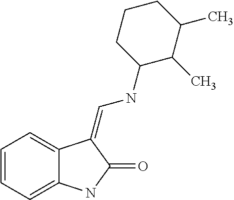 | C 75.52% H 8.20% N 10.36% O 5.92% | C17H22N2O | 270.37769 | ChemDiv | 2 |
| 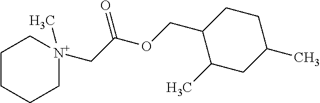 | C 72.29% H 11.42% N 4.96% O 11.33% | C17H32NO2 | 282.45009 | ChemDiv | 2 |
| 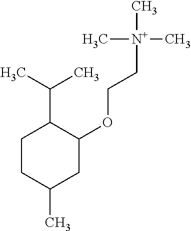 | C 74.32% H 13.31% N 5.78% O 6.60% | C15H32NO | 242.42839 | ChemDiv | 2 |
| 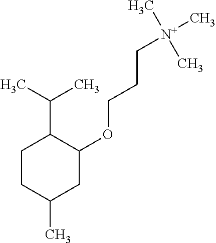 | C 74.94% H 13.36% N 5.46% O 6.24% | C16H34NO | 256.45548 | ChemDiv | 2 |
| 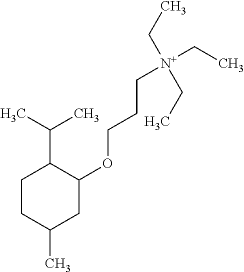 | C 76.44% H 13.51% N 4.69% O 5.36% | C19H40NO | 298.53675 | ChemDiv | 2 |
| 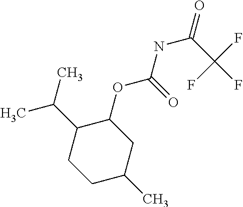 | C 52.88% H 6.83% F 19.03% N 4.74% O 16.25% | C13H20F3NO3 | 295.30445 | ChemDiv | 2 |
| 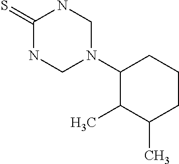 | C 58.11% H 9.31% N 18.48% S 14.10% | C11H21N3S | 227.37412 | ChemDiv | 2 |
| 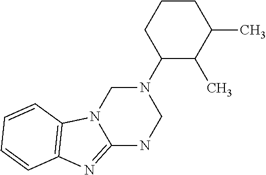 | C 71.79% H 8.51% N 19.70% | C17H24N4 | 284.40763 | ChemDiv | 2 |
| 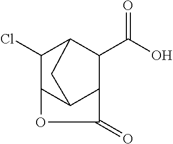 | C 49.90% H 4.19% Cl 16.37% O 29.54% | C9H9ClO4 | 216.62268 | ChemDiv | 2 |
| 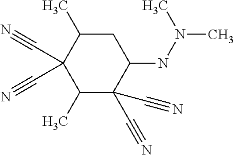 | C 62.20% H 6.71% N 31.09% | C14H18N6 | 270.33976 | ChemDiv | 2 |
| 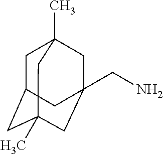 | C 80.76% H 11.99% N 7.24% | C13H23N | 193.33496 | ChemDiv | 2 |
| 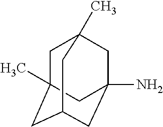 | C 80.38% H 11.81% N 7.81% | C12H21N | 179.30787 | ChemDiv | 2 |
| 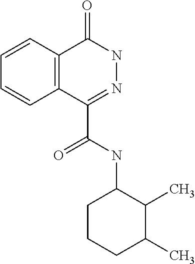 | C 68.21% H 7.07% N 14.04% O 10.69% | C17H21N3O2 | 299.37582 | ChemDiv | 2 |
| 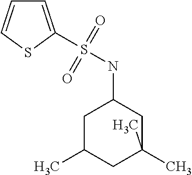 | C 54.32% H 7.36% N 4.87% O 11.13% S 22.31% | C13H21NO2S2 | 287.44582 | ChemDiv | 2 |
| 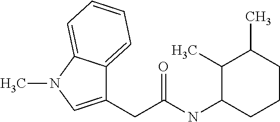 | C 76.47% H 8.78% N 9.39% O 5.36% | C19H26N2O | 298.43187 | ChemDiv | 2 |
| 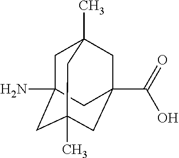 | C 69.92% H 9.48% N 6.27% O 14.33% | C13H21NO2 | 223.31782 | ChemDiv | 2 |
| 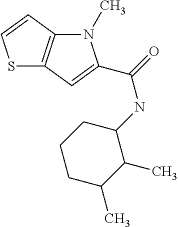 | C 66.17% H 7.64% N 9.65% O 5.51% S 11.04% | C16H22N2OS | 290.43054 | ChemDiv | 2 |
| 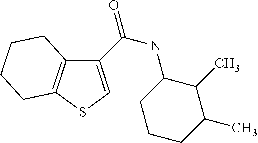 | C 70.06% H 8.65% N 4.81% O 5.49% S 11.00% | C17H25NOS | 291.4589 | ChemDiv | 2 |
| 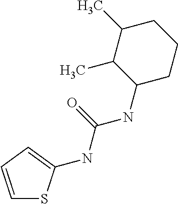 | C 61.87% H 7.99% N 11.10% O 6.34% S 12.70% | C13H20N2OS | 252.38115 | ChemDiv | 2 |
| 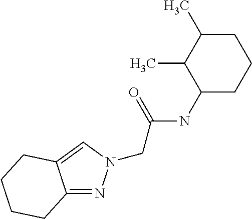 | C 70.55% H 9.40% N 14.52% O 5.53% | C17H27N3O | 289.42424 | ChemDiv | 1 |
| 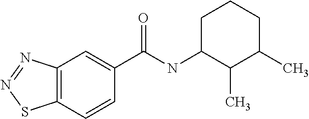 | C 62.25% H 6.62% N 14.52% O 5.53% S 11.08% | C15H19N3OS | 289.40218 | ChemDiv | 1 |
| 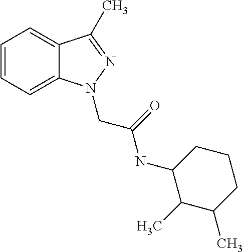 | C 72.21% H 8.42% N 14.03% O 5.34% | C18H25N3O | 299.41945 | ChemDiv | 1 |
| 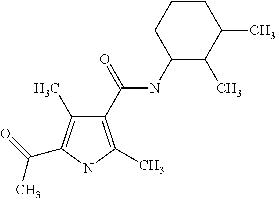 | C 70.31% H 9.02% N 9.65% O 11.02% | C17H26N2O2 | 290.40897 | ChemDiv | 1 |
| 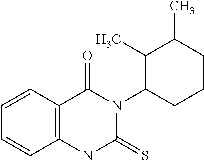 | C 66.63% H 6.99% N 9.71% O 5.55% S 11.12% | C16H20N2OS | 288.4146 | ChemDiv | 1 |
| 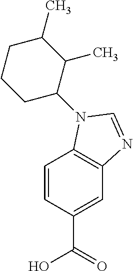 | C 70.56% H 7.40% N 10.29% O 11.75% | C16H20N2O2 | 272.35 | ChemDiv | 1 |
| 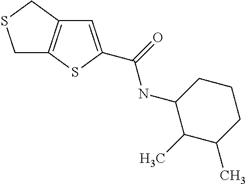 | C 60.98% H 7.16% N 4.74% O 5.41% S 21.70% | C15H21NOS2 | 295.46872 | ChemDiv | 1 |
| 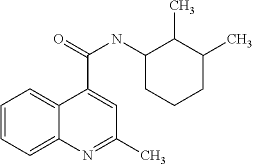 | C 76.99% H 8.16% N 9.45% O 5.40% | C19H24N2O | 296.41593 | ChemDiv | 1 |
| 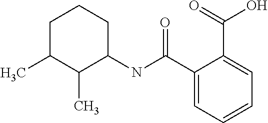 | C 69.79% H 7.69% N 5.09% O 17.43% | C16H21NO3 | 275.35067 | ChemDiv | 2 |
| 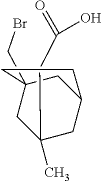 | C 54.37% H 6.67% Br 27.82% O 11.14% | C13H19BrO2 | 287.19918 | Enamine | 1 |
| 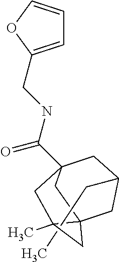 | C 75.22% H 8.77% N 4.87% O 11.13% | C18H25NO2 | 287.40545 | Enamine | 1 |
| 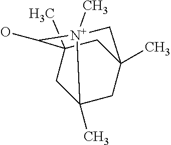 | C 74.59% H 11.08% N 6.69% O 7.64% | C13H23NO | 209.33436 | Enamine | 1 |
| 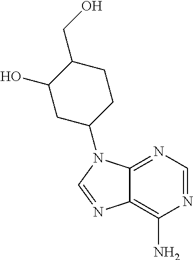 | C 54.74% H 6.51% N 26.60% O 12.15% | C12H17N5O2 | 263.30159 | Enamine | 1 |
| 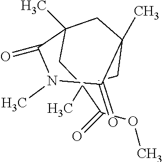 | C 62.90% H 7.92% N 5.24% O 23.94% | C14H21NO4 | 267.32777 | Enamine | 1 |
| 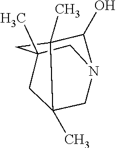 | C 73.80% H 10.84% N 7.17% O 8.19% | C12H21NO | 195.30727 | Enamine | 1 |
| 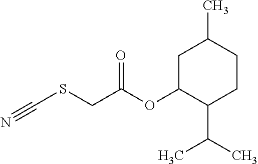 | C 61.14% H 8.29% N 5.48% O 12.53% S 12.56% | C13H21NO2S | 255.38182 | Enamine | 1 |
| 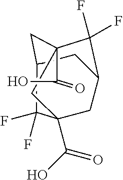 | C 48.66% H 4.08% F 25.65% O 21.60% | C12H12F4O4 | 296.22064 | Enamine | 1 |
| 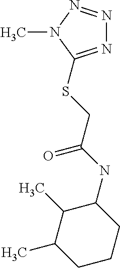 | C 50.86% H 7.47% N 24.71% O 5.65% S 11.31% | C12H21N5OS | 283.39807 | Enamine | 1 |
| 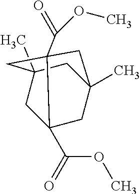 | C 68.55% H 8.63% O 22.83% | C16H24O4 | 280.36728 | Enamine | 1 |
| 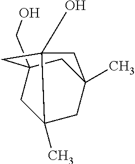 | C 74.95% H 10.78% O 14.26% | C14H24O2 | 224.34618 | Enamine | 1 |
| 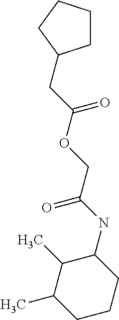 | C 69.12% H 9.89% N 4.74% O 16.25% | C17H29NO3 | 295.42558 | Enamine | 1 |
| 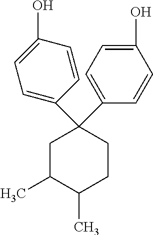 | C 81.04% H 8.16% O 10.80% | C20H24O2 | 296.41308 | Enamine | 1 |
| 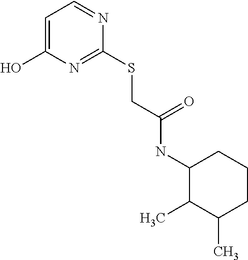 | C 56.92% H 7.17% N 14.22% O 10.83% S 10.85% | C14H21N3O2S | 295.40637 | Enamine | 1 |
| 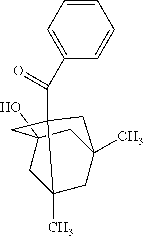 | C 80.24% H 8.51% O 11.25% | C19H24O2 | 284.40193 | Enamine | 1 |
| 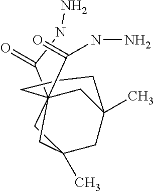 | C 59.98% H 8.63% N 19.98% O 11.41% | C14H24N4O2 | 280.37298 | Enamine | 1 |
| 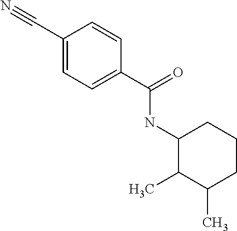 | C 74.97% H 7.86% N 10.93% O 6.24% | C16H20N2O | 256.3506 | Enamine | 1 |
| 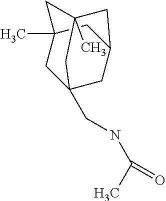 | C 76.55% H 10.71% N 5.95% O 6.80% | C15H25NO | 235.3726 | Enamine | 1 |
| 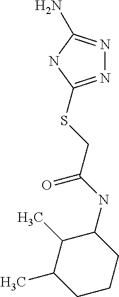 | C 50.86% H 7.47% N 24.71% O 5.65% S 11.31% | C12H21N5OS | 283.39807 | Enamine | 1 |
| 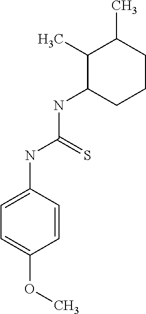 | C 65.71% H 8.27% N 9.58% O 5.47% S 10.96% | C16H24N2OS | 292.44648 | Enamine | 1 |
| 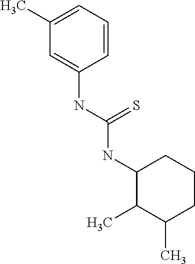 | C 69.52% H 8.75% N 10.13% S 11.60% | C16H24N2S | 276.44708 | Enamine | 1 |
| 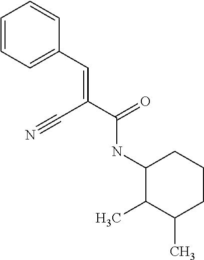 | C 76.56% H 7.85% N 9.92% O 5.67% | C18H22N2O | 282.38884 | Enamine | 1 |
| 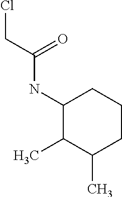 | C 58.96% H 8.91% Cl 17.40% N 6.88% O 7.85% | C10H18ClNO | 203.71406 | Enamine | 1 |
| 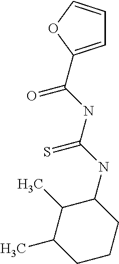 | C 59.97% H 7.19% N 9.99% O 11.41% S 11.44% | C14H20N2O2S | 280.3917 | Enamine | 1 |
| 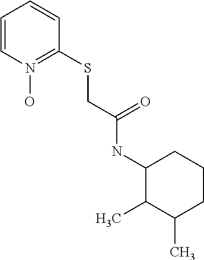 | C 61.19% H 7.53% N 9.51% O 10.87% S 10.89% | C15H22N2O2S | 294.41879 | Enamine | 1 |
| 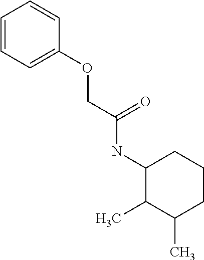 | C 73.53% H 8.87% N 5.36% O 12.24% | C16H23NO2 | 261.36721 | Enamine | 1 |
| 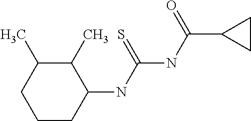 | C 61.38% H 8.72% N 11.01% O 6.29% S 12.60% | C13H22N2OS | 254.39709 | Enamine | 1 |
| 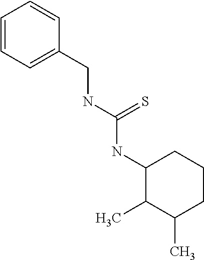 | C 69.52% H 8.75% N 10.13% S 11.60% | C16H24N2S | 276.44708 | Enamine | 1 |
| 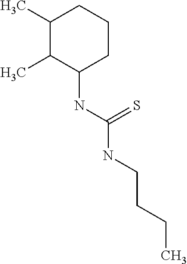 | C 64.41% H 10.81% N 11.56% S 13.23% | C13H26N2S | 242.42957 | Enamine | 1 |
| 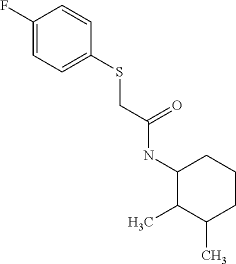 | C 65.05% H 7.51% F 6.43% N 4.74% O 5.42% S 10.85% | C16H22FNOS | 295.42224 | Enamine | 1 |
| 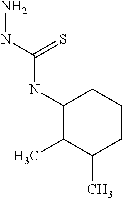 | C 53.69% H 9.51% N 20.87% S 15.93% | C9H19N3S | 201.33588 | Enamine | 1 |
| 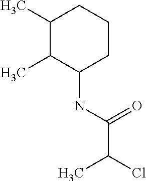 | C 60.68% H 9.26% Cl 16.28% N 6.43% O 7.35% | C11H20ClNO | 217.74115 | Enamine | 1 |
| 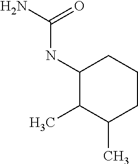 | C 63.49% H 10.66% N 16.45% O 9.40% | C9H18N2O | 170.25661 | Enamine | 1 |
| 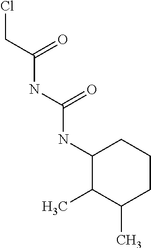 | C 53.55% H 7.76% Cl 14.37% N 11.35% O 12.97% | C11H19ClN2O2 | 246.73928 | Enamine | 1 |
| 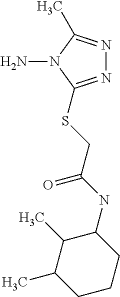 | C 52.50% H 7.79% N 23.55% O 5.38% S 10.78% | C13H23N5OS | 297.42516 | Enamine | 1 |
| 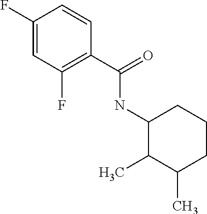 | C 67.40% H 7.16% F 14.21% N 5.24% O 5.99% | C15H19F2NO | 267.32158 | Enamine | 1 |
| 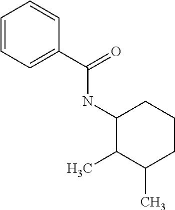 | C 77.88% H 9.15% N 6.05% O 6.92% | C15H21NO | 231.34072 | Enamine | 1 |
| 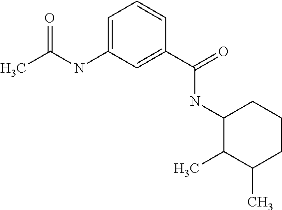 | C 70.80% H 8.39% N 9.71% O 11.10% | C17H24N2O2 | 288.39303 | Enamine | 1 |
| 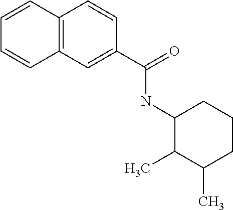 | C 81.10% H 8.24% N 4.98% O 5.69% | C19H23NO | 281.40126 | Enamine | 1 |
| 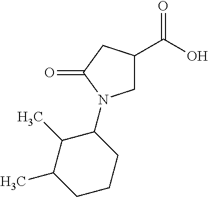 | C 65.25% H 8.85% N 5.85% O 20.06% | C13H21NO3 | 239.31722 | Enamine | 1 |
| 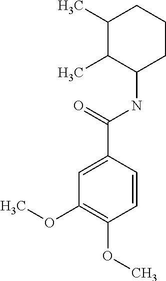 | C 70.07% H 8.65% N 4.81% O 16.47% | C17H25NO3 | 291.3937 | Enamine | 1 |
| 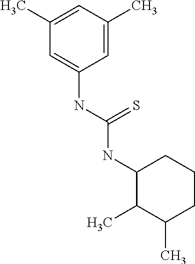 | C 70.30% H 9.02% N 9.64% S 11.04% | C17H26N2S | 290.47417 | Enamine | 1 |
| 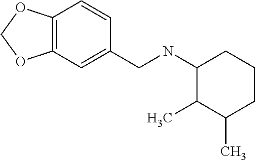 | C 73.53% H 8.87% N 5.36% O 12.24% | C16H23NO2 | 261.36721 | Enamine | 1 |
| 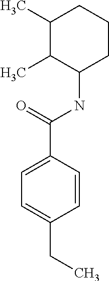 | C 78.72% H 9.71% N 5.40% O 6.17% | C17H25NO | 259.3949 | Enamine | 1 |
| 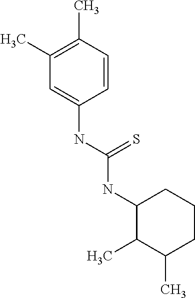 | C 70.30% H 9.02% N 9.64% S 11.04% | C17H26N2S | 290.47417 | Enamine | 1 |
| 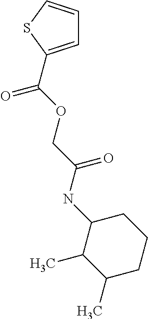 | C 60.99% H 7.17% N 4.74% O 16.25% S 10.85% | C15H21NO3S | 295.40352 | Enamine | 1 |
| 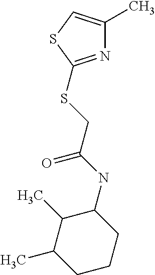 | C 56.34% H 7.43% N 9.39% O 5.36% S 21.49% | C14H22N2OS2 | 298.47224 | Enamine | 1 |
| 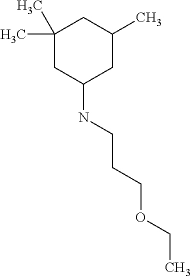 | C 73.95% H 12.86% N 6.16% O 7.04% | C14H29NO | 227.39333 | Enamine | 1 |
| 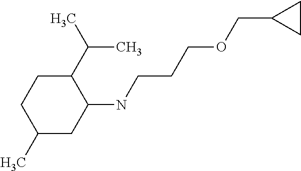 | C 76.34% H 12.44% N 5.24% O 5.98% | C17H33NO | 267.45866 | Enamine | 1 |
| 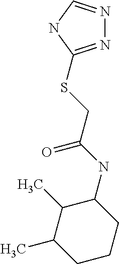 | C 53.70% H 7.51% N 20.88% O 5.96% S 11.95% | C12H20N4OS | 268.3834 | Enamine | 1 |
| 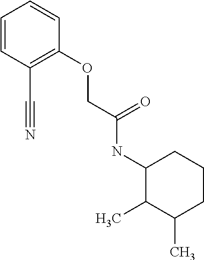 | C 71.30% H 7.74% N 9.78% O 11.17% | C17H22N2O2 | 286.37709 | Enamine | 1 |
| 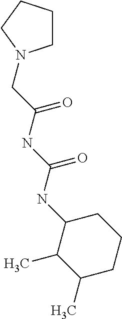 | C 64.03% H 9.67% N 14.93% O 11.37% | C15H27N3O2 | 281.40134 | Enamine | 1 |
| 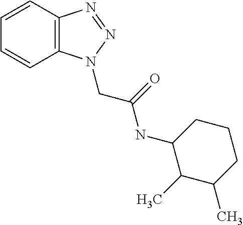 | C 67.11% H 7.74% N 19.56% O 5.59% | C16H22N4O | 286.37994 | Enamine | 1 |
| 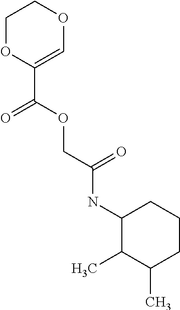 | C 60.59% H 7.80% N 4.71% O 26.90% | C15H23NO5 | 297.35426 | Enamine | 1 |
| 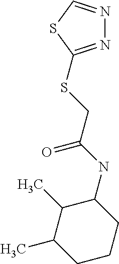 | C 50.50% H 6.71% N 14.72% O 5.61% S 22.47% | C12H19N3OS2 | 285.43273 | Enamine | 1 |
| 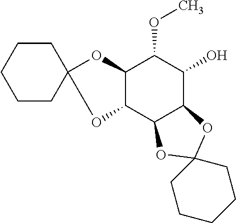 | C 64.39% H 8.53% O 27.08% | C19H30O6 | 354.44735 | Sigma- Aldrich | 3 |
| 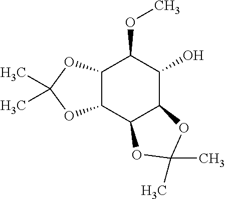 | C 56.92% H 8.08% O 34.99% | C13H22O6 | 274.31669 | Sigma- Aldrich | 2 |
| 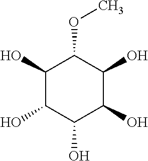 | C 43.30% H 7.27% O 49.44% | C7H14O6 | 194.18603 | Sigma- Aldrich | 3 |
| 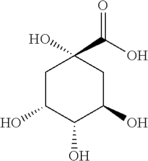 | C 43.75% H 6.29% O 49.95% | C7H12O6 | 192.17009 | Sigma- Aldrich | 3 |
| 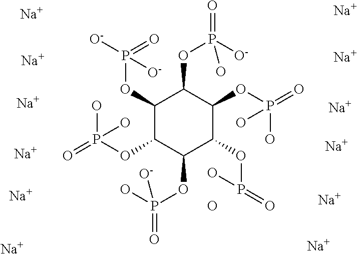 | C 7.80% H 0.65% Na 29.86% O 41.56% P 20.12% | C6H6Na12O24P6 | 923.82072 | Sigma- Aldrich | 2 |
| 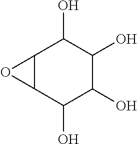 | C 44.45% H 6.22% O 49.34% | C6H10O5 | 162.1436 | Sigma- Aldrich | 1 |
| 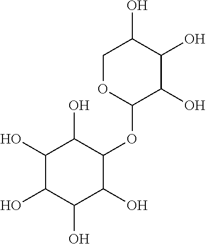 | C 42.31% H 6.46% O 51.23% | C11H20O10 | 312.27605 | Sigma- Aldrich | 3 |
| 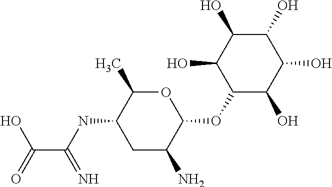 | C 42.32% H 6.85% N 10.57% O 40.26% | C14H27N3O10 | 397.38539 | Sigma- Aldrich | 2 |
| 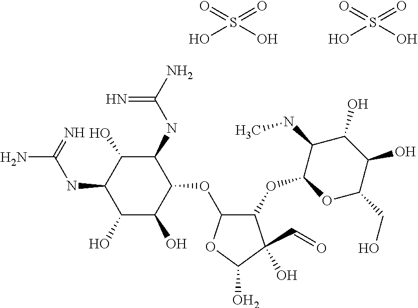 | C 32.43% H 5.57% N 12.61% O 41.14% S 8.25% | C21H43N7O20S2 | 777.73976 | Sigma- Aldrich | 2 |
| 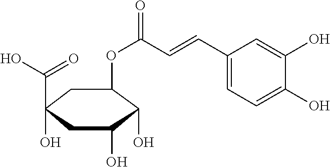 | C 54.24% H 5.12% O 40.64% | C16H18O9 | 354.31646 | Sigma- Aldrich | 2 |
| 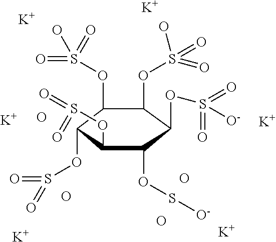 | C 8.11% H 0.68% K 26.39% O 43.19% S 21.64% | C6H6K6O24S6 | 889.09632 | Sigma- Aldrich | 4 |
| 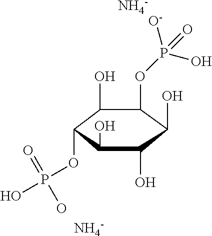 | C 19.26% H 5.39% N 7.49% O 51.31% P 16.56% | C6H20N2O12P2 | 374.1801 | Sigma- Aldrich | 12 |
| 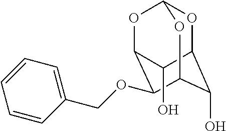 | C 60.00% H 5.75% O 34.25% | C14H16O6 | 280.28002 | AMRI | 3 |
| 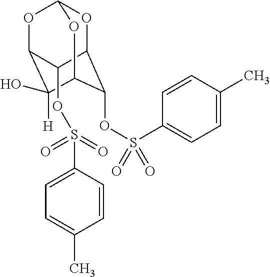 | C 50.60% H 4.45% O 32.09% S 12.86% | C21H22O10S2 | 498.53149 | AMRI | 3 |
| 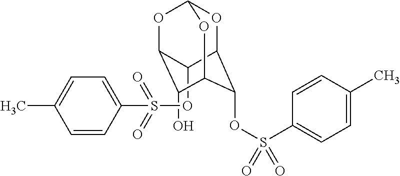 | C 50.60% H 4.45% O 32.09% S 12.86% | C21H22O10S2 | 498.53149 | AMRI | 4 |
| 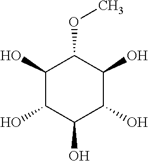 | C 43.30% H 7.27% O 49.44% | C7H14O6 | 194.18603 | Dalton | 48 |
| 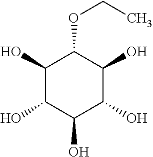 | C 46.15% H 7.75% O 46.10% | C8H16O6 | 208.21312 | Dalton | 14 |
| 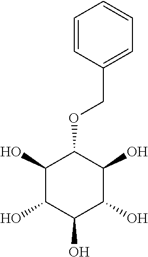 | C 57.77% H 6.71% O 35.52% | C13H18O6 | 270.28481 | Dalton | 3 |
| 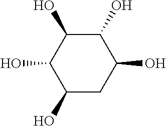 | C 43.90% H 7.37% O 48.73% | C6H12O5 | 164.15954 | J. McLaurin | 15 |
| 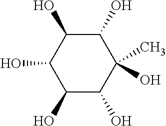 | C 43.30% H 7.27% O 49.44% | C7H14O6 | 194.18603 | J. McLaurin | 22 |
| 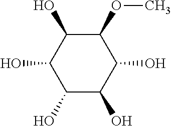 | C 43.30% H 7.27% O 49.44% | C7H14O6 | 194.18603 | J. McLaurin | 5 |
| 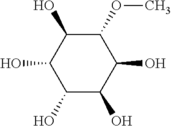 | C 43.30% H 7.27% O 49.44% | C7H14O6 | 194.18603 | J. McLaurin | 2 |
| 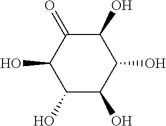 | C 40.45% H 5.66% O 53.89% | C6H10O6 | 178.143 | J. McLaurin | 11 |
| 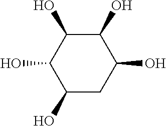 | C 43.90% H 7.37% O 48.73% | C6H12O5 | 164.15954 | J. McLaurin | 4 |
| 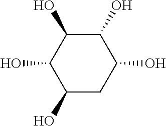 | C 43.90% H 7.37% O 48.73% | C6H12O5 | 164.15954 | J. McLaurin | 5 |
| 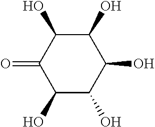 | C 40.45% H 5.66% O 53.89% | C6H10O6 | 178.143 | J. McLaurin | 7 |
| 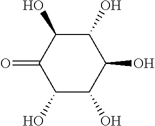 | C 40.45% H 5.66% O 53.89% | C6H10O6 | 178.143 | J. McLaurin | 5 |
| 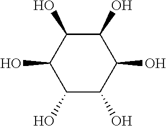 | C 40.00% H 6.71% O 53.28% | C6H12O6 | 180.15894 | J. McLaurin | 10 |
| 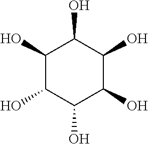 | C 40.00% H 6.71% O 53.28% | C6H12O6 | 180.15894 | Sigma- Aldrich | 4 |
| 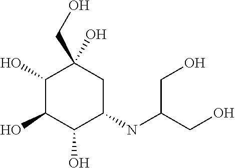 | C 44.94% H 7.92% N 5.24% O 41.90% | C10H21NO7 | 267.28137 | Molcan | 3 |
| 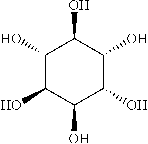 | C 40.00% H 6.71% O 53.28% | C6H12O6 | 180.15894 | IRL | 2 |
| 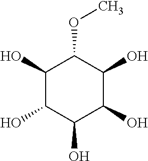 | C 43.30% H 7.27% O 49.44% | C7H14O6 | 194.18603 | IRL | 4 |
| 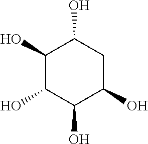 | C 43.90% H 7.37% O 48.73% | C6H12O5 | 164.15954 | IRL | 2 |
| 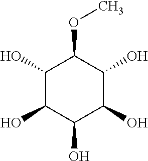 | C 43.30% H 7.27% O 49.44% | C7H14O6 | 194.18603 | IRL | 3 |
| 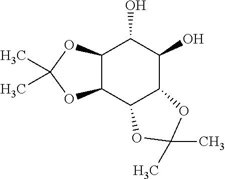 | C 55.37% H 7.75% O 36.88% | C12H20O6 | 260.2896 | IRL | 2 |
| 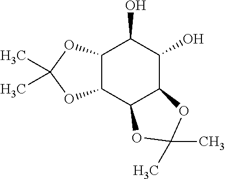 | C 55.37% H 7.75% O 36.88% | C12H20O6 | 260.2896 | IRL | 2 |
| 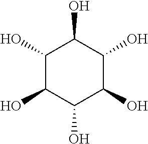 | C 40.00% H 6.71% O 53.28% | C6H12O6 | 180.15894 | Sigma- Aldrich | 89 |
| 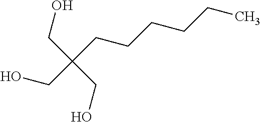 | C 63.12% H 11.65% O 25.22% | C10H22O3 | 190.28504 | ChemDiv | 0 |
| 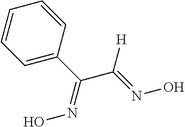 | C 58.53% H 4.91% N 17.06% O 19.49% | C8H8N2O2 | 164.16516 | ChemDiv | 0 |
| 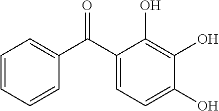 | C 67.82% H 4.38% O 27.80% | C13H10O4 | 230.22225 | ChemDiv | 0 |
| 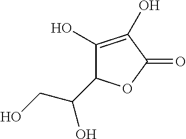 | C 40.92% H 4.58% O 54.50% | C6H8O6 | 176.12706 | ChemDiv | 0 |
| 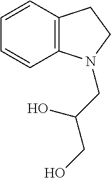 | C 68.37% H 7.82% N 7.25% O 16.56% | C11H15NO2 | 193.2477 | ChemDiv | 0 |
| 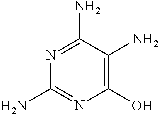 | C 34.04% H 5.00% N 49.62% O 11.34% | C4H7N5O | 141.13329 | ChemDiv | 0 |
| 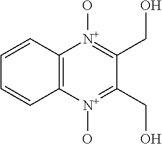 | C 54.06% H 4.54% N 12.61% O 28.80% | C10H10N2O4 | 222.2022 | ChemDiv | 0 |
| 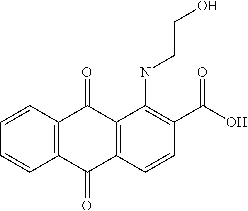 | C 65.59% H 4.21% N 4.50% O 25.70% | C17H13NO5 | 311.29686 | ChemDiv | 0 |
| 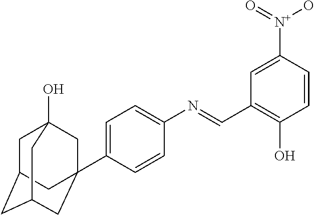 | C 70.39% H 6.16% N 7.14% O 16.31% | C23H24N2O4 | 392.45873 | ChemDiv | 0 |
| 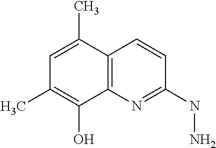 | C 65.01% H 6.45% N 20.67% O 7.87% | C11H13N3O | 203.24576 | ChemDiv | 0 |
| 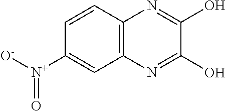 | C 46.39% H 2.43% N 20.29% O 30.89% | C8H5N3O4 | 207.14675 | ChemDiv | 0 |
| 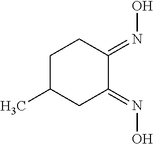 | C 53.83% H 7.74% N 17.94% O 20.49% | C7H12N2O2 | 156.18589 | ChemDiv | 0 |
| 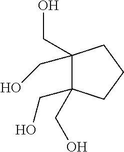 | C 56.82% H 9.54% O 33.64% | C9H18O4 | 190.24141 | ChemDiv | 0 |
| 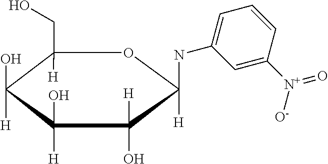 | C 48.00% H 5.37% N 9.33% O 37.30% | C12H16N2O7 | 300.27052 | ChemDiv | 0 |
| 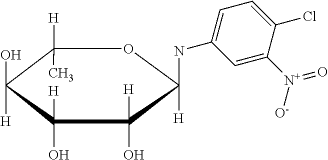 | C 45.22% H 4.74% Cl 11.12% N 8.79% O 30.12% | C12H15ClN2O6 | 319.71615 | ChemDiv | 0 |
| 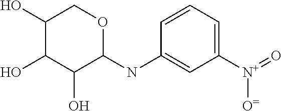 | C 48.89% H 5.22% N 10.37% O 35.52% | C11H14N2O6 | 270.24403 | ChemDiv | 0 |
| 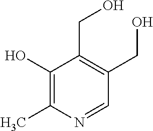 | C 56.80% H 6.55% N 8.28% O 28.37% | C8H11NO3 | 169.18177 | ChemDiv | 0 |
| 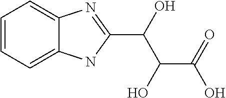 | C 54.06% H 4.54% N 12.61% O 28.80% | C10H10N2O4 | 222.2022 | ChemDiv | 0 |
| 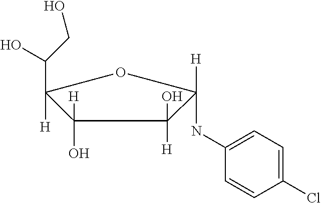 | C 49.75% H 5.57% Cl 12.24% N 4.83% O 27.61% | C12H16ClNO5 | 289.71802 | ChemDiv | 0 |
| 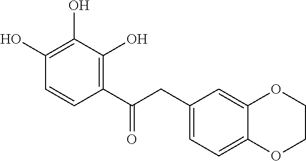 | C 63.57% H 4.67% O 31.76% | C16H14O6 | 302.28638 | ChemDiv | 0 |
| 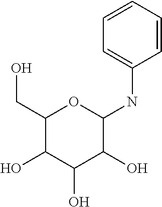 | C 56.46% H 6.71% N 5.49% O 31.34% | C12H17NO5 | 255.27299 | ChemDiv | 0 |
| 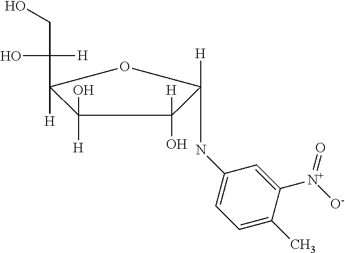 | C 49.68% H 5.77% N 8.91% O 35.63% | C13H18N2O7 | 314.29761 | ChemDiv | 0 |
| 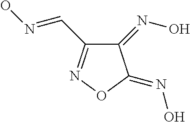 | C 28.08% H 1.77% N 32.75% O 37.41% | C4H3N4O4 | 171.09291 | ChemDiv | 0 |
| 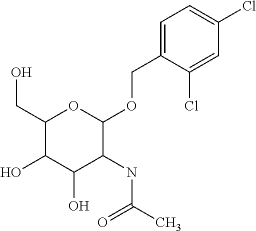 | C 47.38% H 5.04% Cl 18.65% N 3.68% O 25.25% | C15H19Cl2NO6 | 380.22778 | ChemDiv | 0 |
| 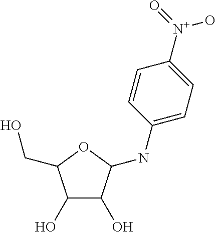 | C 48.89% H 5.22% N 10.37% O 35.52% | C11H14N2O6 | 270.24403 | ChemDiv | 0 |
| 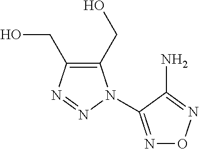 | C 33.97% H 3.80% N 39.61% O 22.62% | C6H8N6O3 | 212.16906 | ChemDiv | 0 |
| 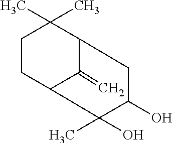 | C 74.24% H 10.54% O 15.21% | C13H22O2 | 210.31909 | ChemDiv | 0 |
| 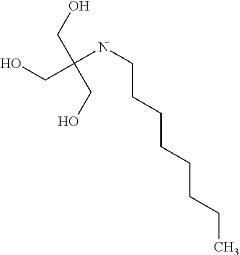 | C 61.77% H 11.66% N 6.00% O 20.57% | C12H27NO3 | 233.35389 | ChemDiv | 0 |
| 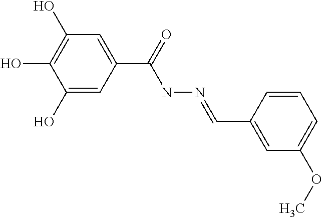 | C 59.60% H 4.67% N 9.27% O 26.46% | C15H14N2O5 | 302.28923 | ChemDiv | 0 |
| 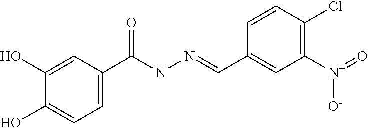 | C 50.09% H 3.00% Cl 10.56% N 12.52% O 23.83% | C14H10ClN3O5 | 335.7059 | ChemDiv | 0 |
| 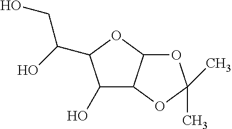 | C 49.09% H 7.32% O 43.59% | C9H16O6 | 220.22427 | ChemDiv | 0 |
| 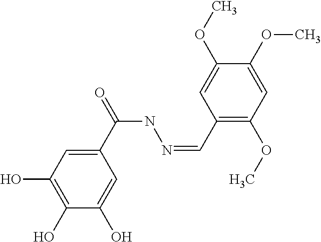 | C 56.35% N 5.01% N 7.73% O 30.91% | C17H18N2O7 | 362.34221 | ChemDiv | 0 |
| 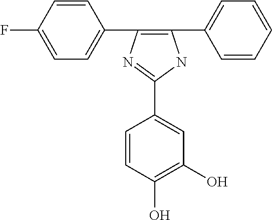 | C 72.82% H 4.37% F 5.49% N 8.09% O 9.24% | C21H15FN2O2 | 346.3643 | ChemDiv | 0 |
| 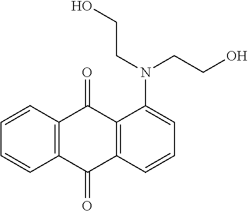 | C 69.44% H 5.50% N 4.50% O 20.56% | C18H17NO4 | 311.34049 | ChemDiv | 0 |
| 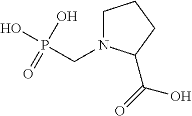 | C 34.46% H 5.78% N 6.70% O 38.25% P 14.81% | C6H12NO5P | 209.14004 | ChemDiv | 0 |
| 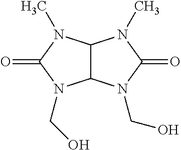 | C 41.74% H 6.13% N 24.34% O 27.80% | C8H14N4O4 | 230.22518 | ChemDiv | 0 |
| 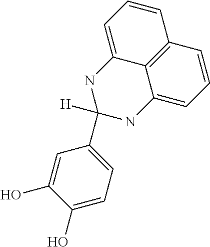 | C 73.37% H 5.07% N 10.07% O 11.50% | C17H14N2O2 | 278.31333 | ChemDiv | 0 |
| 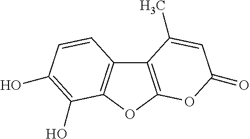 | C 62.07% H 3.47% O 34.45% | C12H8O5 | 232.19456 | ChemDiv | 0 |
| 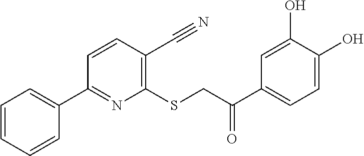 | C 66.28% H 3.89% N 7.73% O 13.24% S 8.85% | C20H14N2O3S | 362.41018 | ChemDiv | 0 |
| 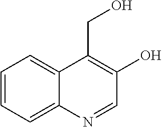 | C 68.56% H 5.18% N 8.00% O 18.27% | C10H9NO2 | 175.18873 | ChemDiv | 0 |
| 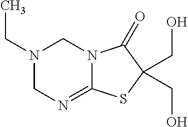 | C 44.07% H 6.16% N 17.13% O 19.57% S 13.07% | C9H15N3O3S | 245.3022 | ChemDiv | 0 |
| 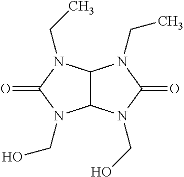 | C 46.50% H 7.02% N 21.69% O 24.78% | C10H18N4O4 | 258.27936 | ChemDiv | 0 |
| 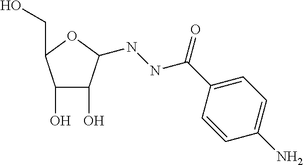 | C 50.88% H 6.05% N 14.83% O 28.24% | C12H17N3O5 | 283.28639 | ChemDiv | 0 |
| 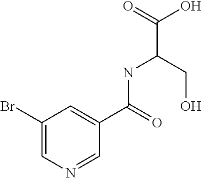 | C 37.39% H 3.14% Br 27.64% N 9.69% O 22.14% | C9H9BrN2O4 | 289.08708 | ChemDiv | 0 |
| 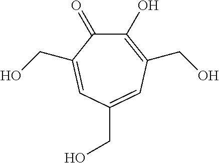 | C 56.60% H 5.70% O 37.70% | C10H12O5 | 212.20414 | ChemDiv | 0 |
| 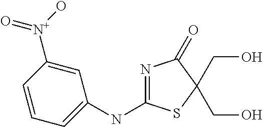 | C 44.44% H 3.73% N 14.13% O 26.91% S 10.79% | C11H11N3O5S | 297.29142 | ChemDiv | 0 |
| 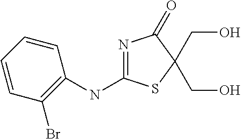 | C 39.89% H 3.35% Br 24.13% N 8.46% O 14.49% S 9.68% | C11H11BrN2O3S | 331.18992 | ChemDiv | 0 |
| 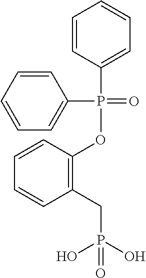 | C 58.77% H 4.67% O 20.60% P 15.95% | C19H18O5P2 | 388.29991 | ChemDiv | 0 |
| 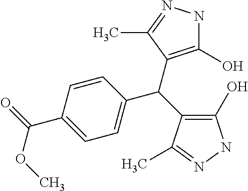 | C 59.64% H 5.30% N 16.37% O 18.69% | C17H18N4O4 | 342.35741 | ChemDiv | 0 |
| 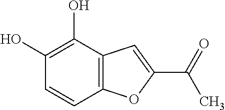 | C 62.50% H 4.20% O 33.30% | C10H8O4 | 192.17286 | ChemDiv | 0 |
| 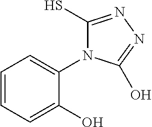 | C 45.93% H 3.37% N 20.08% O 15.29% S 15.32% | C8H7N3O2S | 209.22789 | ChemDiv | 0 |
| 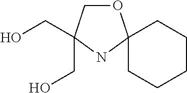 | C 59.68% H 9.52% N 6.96% O 23.85% | C10H19NO3 | 201.26783 | ChemDiv | 0 |
| 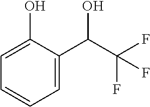 | C 50.01% H 3.67% F 29.66% O 16.65% | C8H7F3O2 | 192.13899 | ChemDiv | 0 |
| 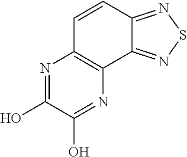 | C 43.64% H 1.83% N 25.44% O 14.53% S 14.56% | C8H4N4O2S | 220.21068 | ChemDiv | 0 |
| 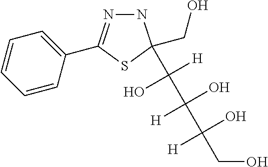 | C 49.67% H 5.77% N 8.91% O 25.45% S 10.20% | C13H18N2O5S | 314.36281 | ChemDiv | 0 |
| 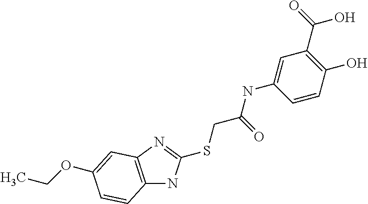 | C 55.81% H 4.42% N 10.85% O 20.65% S 8.28% | C18H17N3O5S | 387.41729 | ChemDiv | 0 |
| 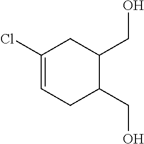 | C 54.40% H 7.42% Cl 20.07% O 18.11% | C8H13ClO2 | 176.64461 | ChemDiv | 0 |
| 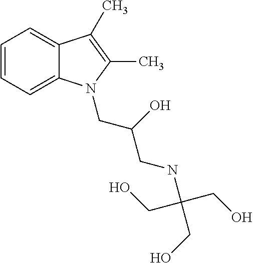 | C 63.33% H 8.13% N 8.69% O 19.85% | C17H26N2O4 | 322.40777 | ChemDiv | 0 |
| 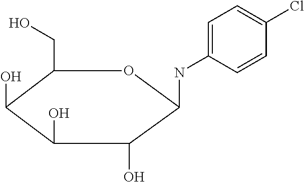 | C 49.75% H 5.57% Cl 12.24% N 4.83% O 27.61% | C12H16ClNO5 | 289.71802 | ChemDiv | 0 |
| 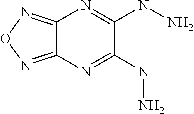 | C 26.38% H 3.32% N 61.52% O 8.78% | C4H6N8O | 182.14542 | ChemDiv | 0 |
| 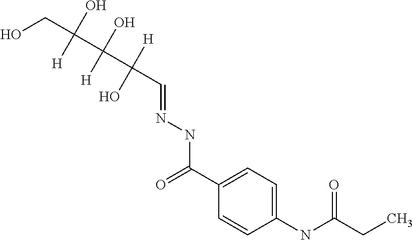 | C 53.09% H 6.24% N 12.38% O 28.29% | C15H21N3O6 | 339.35112 | ChemDiv | 0 |
| 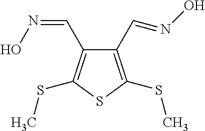 | C 36.62% H 3.84% N 10.68% O 12.20% S 36.66% | C8H10N2O2S3 | 262.3731 | ChemDiv | 0 |
| 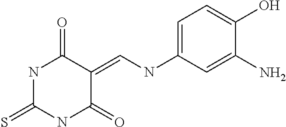 | C 47.48% H 3.62% N 20.13% O 17.25% S 11.52% | C11H10N4O3S | 278.29135 | ChemDiv | 0 |
| 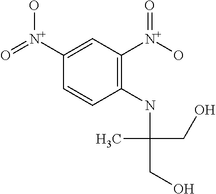 | C 44.28% H 4.83% N 15.49% O 35.39% | C10H13N3O6 | 271.23161 | ChemDiv | 0 |
| 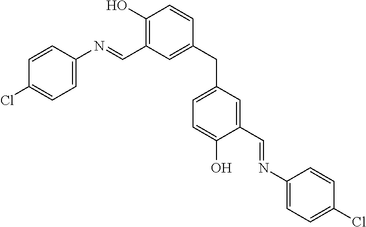 | C 68.22% H 4.24% Cl 14.92% N 5.89% O 6.73% | C27H20Cl2N2O2 | 475.37865 | ChemDiv | 0 |
| 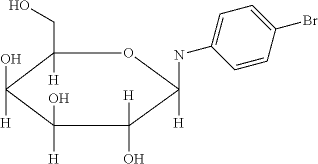 | C 43.13% H 4.83% Br 23.91% N 4.19% O 23.94% | C12H16BrNO5 | 334.16902 | ChemDiv | 0 |
| 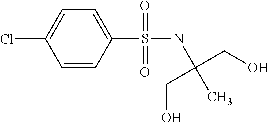 | C 42.94% H 5.04% Cl 12.67% N 5.01% O 22.88% S 11.46% | C10H14ClNO4S | 279.74439 | ChemDiv | 0 |
| 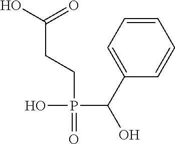 | C 49.19% H 5.37% O 32.76% P 12.68% | C10H13O5P | 244.18591 | ChemDiv | 0 |
| 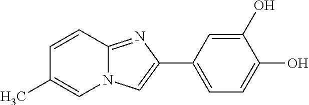 | C 69.99% H 5.03% N 11.66% O 13.32% | C14H12N2O2 | 240.26394 | ChemDiv | 0 |
| 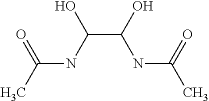 | C 40.91% H 6.87% N 15.90% O 36.33% | C6H12N2O4 | 176.17354 | ChemDiv | 0 |
| 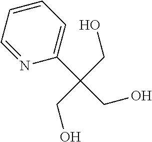 | C 59.00% H 7.15% N 7.65% O 26.20% | C9H13NO3 | 183.20886 | ChemDiv | 0 |
| 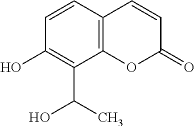 | C 64.08% H 4.89% O 31.04% | C11H10O4 | 206.19995 | ChemDiv | 0 |
| 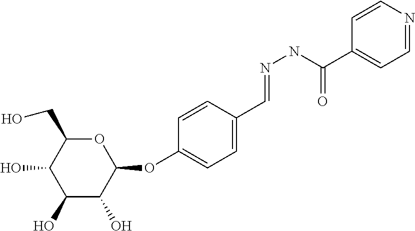 | C 56.57% H 5.25% N 10.42% O 27.76% | C19H21N3O7 | 403.39512 | ChemDiv | 0 |
| 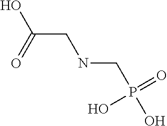 | C 21.31% H 4.77% N 8.28% O 47.31% P 18.32% | C3H8NO5P | 169.07471 | ChemDiv | 0 |
| 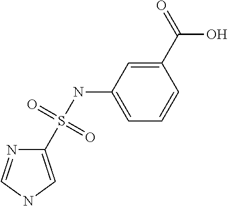 | C 44.94% H 3.39% N 15.72% O 23.95% S 12.00% | C10H9N3O4S | 267.26493 | ChemDiv | 0 |
| 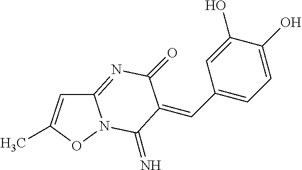 | C 58.95% H 3.89% N 14.73% O 22.43% | C14H11N3O4 | 285.26147 | ChemDiv | 0 |
| 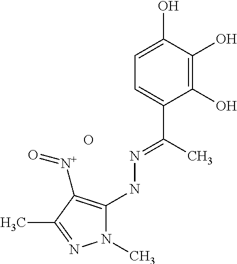 | C 48.60% H 4.71% N 21.80% O 24.90% | C13H15N5O5 | 321.295 | ChemDiv | 0 |
| 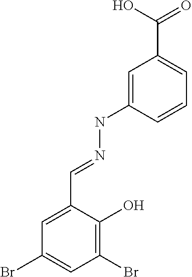 | C 40.61% H 2.43% Br 38.60% N 6.77% O 11.59% | C14H10Br2N2O3 | 414.0554 | ChemDiv | 0 |
| 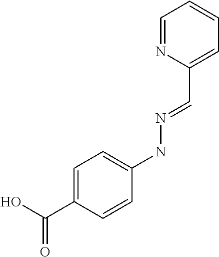 | C 64.72% H 4.60% N 17.42% O 13.26% | C13H11N3O2 | 241.25152 | ChemDiv | 0 |
| 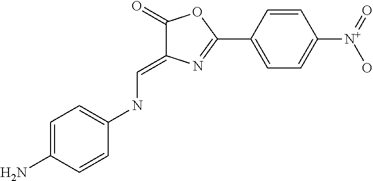 | C 59.26% H 3.73% N 17.28% O 19.73% | C16H12N4O4 | 324.29844 | ChemDiv | 0 |
| 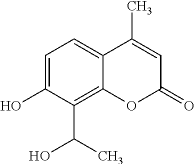 | C 65.45% H 5.49% O 29.06% | C12H12O4 | 220.22704 | ChemDiv | 0 |
| 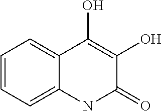 | C 61.02% H 3.98% N 7.91% O 27.09% | C9H7NO3 | 177.16104 | ChemDiv | 0 |
| 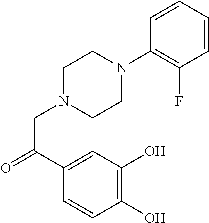 | C 65.44% H 5.80% F 5.75% N 8.48% O 14.53% | C18H19FN2O3 | 330.36213 | ChemDiv | 0 |
| 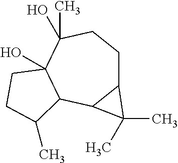 | C 75.58% H 10.99% O 13.42% | C15H26O2 | 238.37327 | ChemDiv | 0 |
| 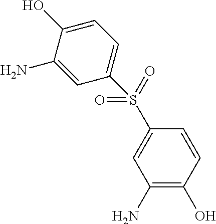 | C 51.42% H 4.32% N 9.99% O 22.83% S 11.44% | C12H12N2O4S | 280.30444 | ChemDiv | 0 |
| 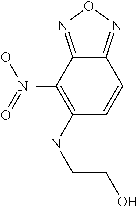 | C 42.86% H 3.60% N 24.99% O 28.55% | C8H8N4O4 | 224.17736 | ChemDiv | 0 |
| 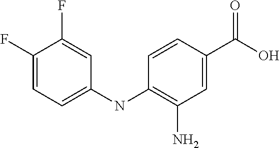 | C 59.09% H 3.81% F 14.38% N 10.60% O 12.11% | C13H10F2N2O2 | 264.23365 | ChemDiv | 0 |
| 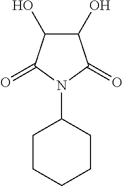 | C 56.33% H 7.09% N 6.57% O 30.01% | C10H15NO4 | 213.23535 | ChemDiv | 0 |
| 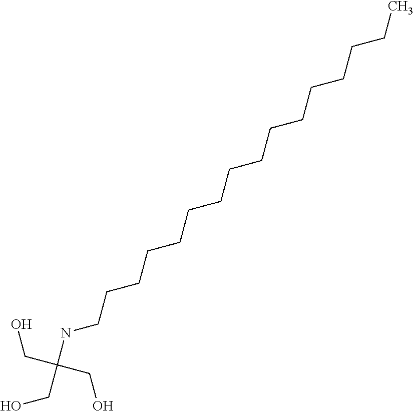 | C 69.51% H 12.54% N 4.05% O 13.89% | C20H43NO3 | 345.57061 | ChemDiv | 0 |
| 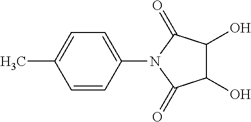 | C 59.73% H 5.01% N 6.33% O 28.93% | C11H11NO4 | 221.21462 | ChemDiv | 0 |
| 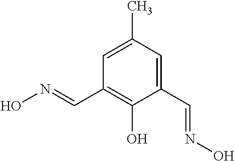 | C 55.67% H 5.19% N 14.43% O 24.72% | C9H10N2O3 | 194.19165 | ChemDiv | 0 |
| 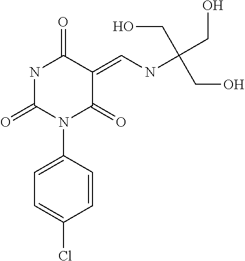 | C 48.72% H 4.36% Cl 9.59% N 11.36% O 25.96% | C15H16ClN3O6 | 369.76427 | ChemDiv | 0 |
| 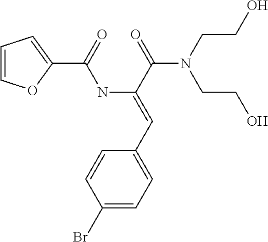 | C 51.08% H 4.52% Br 18.88% N 6.62% O 18.90% | C18H19BrN2O5 | 423.26653 | ChemDiv | 0 |
| 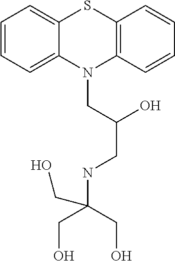 | C 60.62% H 6.43% N 7.44% O 17.00% S 8.52% | C19H24N2O4S | 376.47813 | ChemDiv | 0 |
| 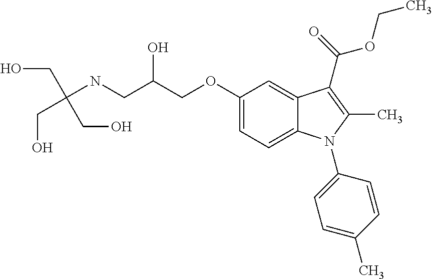 | C 64.18% H 7.04% N 5.76% O 23.02% | C26H34N2O7 | 486.57008 | ChemDiv | 0 |
| 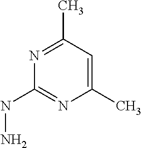 | C 52.16% H 7.30% N 40.55% | C6H10N4 | 138.1734 | Specs | 1 |
| 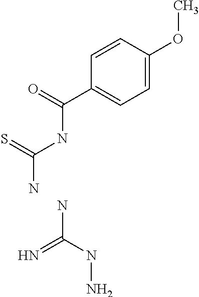 | C 42.54% H 5.00% N 29.77% O 11.33% S 11.36% | C10H14N6O2S | 282.32608 | Specs | 1 |
| 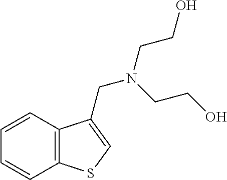 | C 62.12% H 6.82% N 5.57% O 12.73% S 12.76% | C13H17NO2S | 251.34994 | Specs | 1 |
| 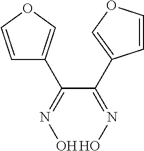 | C 54.55% H 3.66% N 12.72% O 29.07% | C10H8N2O4 | 220.18626 | Specs | 1 |
| 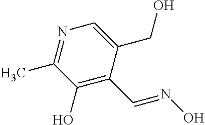 | C 52.74% H 5.53% N 15.38% O 26.35% | C8H10N2O3 | 182.1805 | Specs | 1 |
| 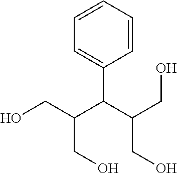 | C 64.98% H 8.39% O 26.63% | C13H20O4 | 240.30195 | Specs | 1 |
| 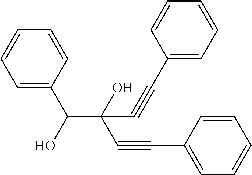 | C 85.18% H 5.36% O 9.46% | C24H18O2 | 338.40986 | Specs | 1 |
| 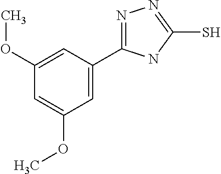 | C 50.62% H 4.67% N 17.71% O 13.49% S 13.51% | C10H11N3O2S | 237.28207 | Specs | 1 |
| 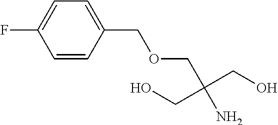 | C 57.63% H 7.04% F 8.29% N 6.11% O 20.94% | C11H16FNO3 | 229.25347 | Specs | 1 |
| 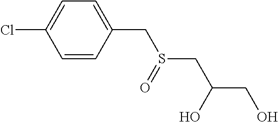 | C 48.29% H 5.27% Cl 14.25% O 19.30% S 12.89% | C10H13ClO3S | 248.73031 | Specs | 1 |
| 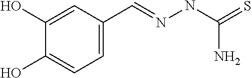 | C 45.49% H 4.29% N 19.89% O 15.15% S 15.18% | C8H9N3O2S | 211.24383 | Specs | 1 |
| 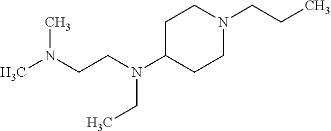 | C 69.65% H 12.94% N 17.41% | C14H31N3 | 241.42327 | Specs | 1 |
| 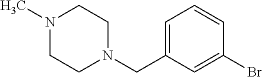 | C 53.54% H 6.37% Br 29.68% N 10.41% | C12H17BrN2 | 269.18669 | Specs | 1 |
| 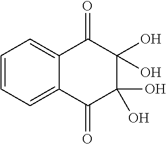 | C 53.58% H 3.60% O 42.82% | C10H8O6 | 224.17166 | Specs | 1 |
| 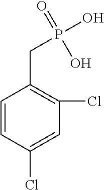 | C 34.89% H 2.93% Cl 29.42% O 19.92% P 12.85% | C7H7Cl2O3P | 241.01184 | Specs | 1 |
| 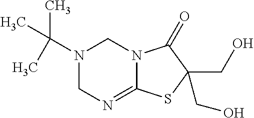 | C 48.33% H 7.01% N 15.37% O 17.56% S 11.73% | C11H19N3O3S | 273.35638 | Specs | 1 |
| 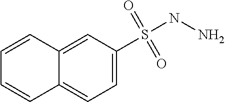 | C 54.04% H 4.54% N 12.60% O 14.40% S 14.43% | C10H10N2O2S | 222.2674 | Specs | 1 |
| 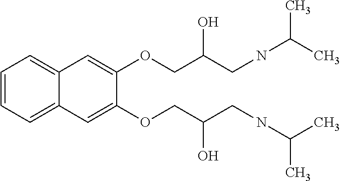 | C 67.66% H 8.78% N 7.17% O 16.39% | C22H34N2O4 | 390.52728 | Specs | 1 |
| 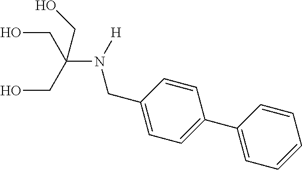 | C 71.06% H 7.37% N 4.87% O 16.70% | C17H21NO3 | 287.36182 | Specs | 1 |
| 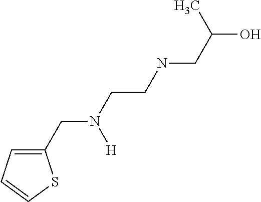 | C 56.04% H 8.47% N 13.07% O 7.46% S 14.96% | C10H18N2OS | 214.33176 | Specs | 1 |
| 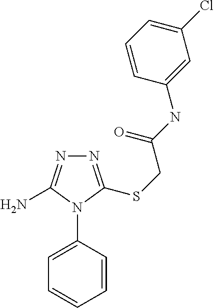 | C 53.41% H 3.92% Cl 9.85% N 19.46% O 4.45% S 8.91% | C16H14ClN5OS | 359.83988 | Specs | 1 |
| 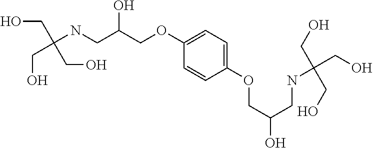 | C 51.71% H 7.81% N 6.03% O 34.44% | C20H36N2O10 | 464.51732 | ChemBridge | 2 |
| 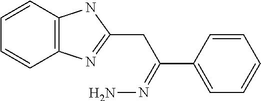 | C 71.98% H 5.64% N 22.38% | C15H14N4 | 250.30563 | ChemBridge | 2 |
| 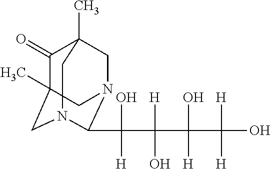 | C 55.99% H 8.05% N 9.33% O 26.63% | C14H24N2O5 | 300.35778 | ChemBridge | 2 |
| 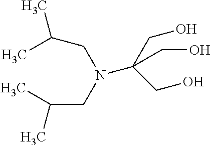 | C 61.77% H 11.66% N 6.00% O 20.57% | C12H27NO3 | 233.35389 | ChemBridge | 2 |
| 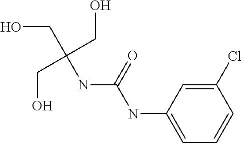 | C 48.10% H 5.50% Cl 12.91% N 10.20% O 23.30% | C11H15ClN2O4 | 274.7062 | ChemBridge | 2 |
| 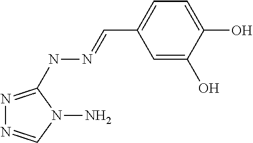 | C 46.15% H 4.30% N 35.88% O 13.66% | C9H10N6O2 | 234.21905 | ChemBridge | 2 |
| 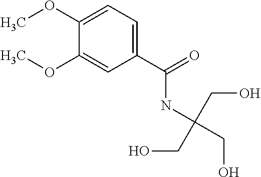 | C 54.73% H 6.71% N 4.91% O 33.65% | C13H19NO6 | 285.29948 | ChemBridge | 2 |
| 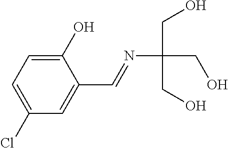 | C 50.88% H 5.43% Cl 13.65% N 5.39% O 24.64% | C11H14ClNO4 | 259.69153 | ChemBridge | 2 |
| 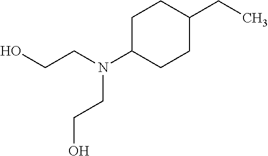 | C 66.93% H 11.70% N 6.50% O 14.86% | C12H25NO2 | 215.33855 | ChemBridge | 2 |
| 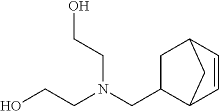 | C 68.21% H 10.02% N 6.63% O 15.14% | C12H21NO2 | 211.30667 | ChemBridge | 2 |
| 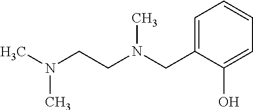 | C 69.19% H 9.68% N 13.45% O 7.68% | C12H20N2O | 208.306 | ChemBridge | 2 |
| 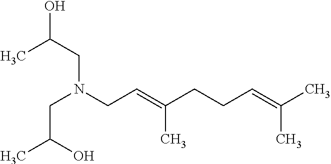 | C 71.33% H 11.60% N 5.20% O 11.88% | C16H31NO2 | 269.43097 | ChemBridge | 2 |
| 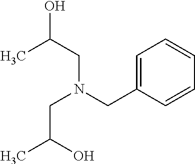 | C 69.92% H 9.48% N 6.27% O 14.33% | C13H21NO2 | 223.31782 | ChemBridge | 2 |
| 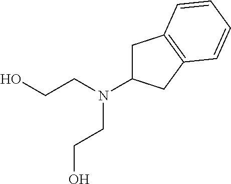 | C 70.56% H 8.65% N 6.33% O 14.46% | C13H19NO2 | 221.30188 | ChemBridge | 2 |
| 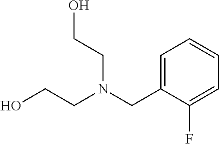 | C 61.96% H 7.56% F 8.91% N 6.57% O 15.01% | C11H16FNO2 | 213.25407 | ChemBridge | 2 |
| 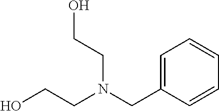 | C 67.66% H 8.78% N 7.17% O 16.39% | C11H17NO2 | 195.26364 | ChemBridge | 2 |
| 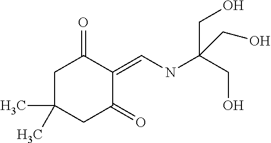 | C 57.55% H 7.80% N 5.16% O 29.48% | C13H21NO5 | 271.31602 | ChemBridge | 2 |
| 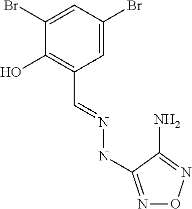 | C 28.67% H 1.87% Br 42.39% N 18.58% O 8.49% | C9H7Br2N5O2 | 376.99644 | ChemBridge | 2 |
| 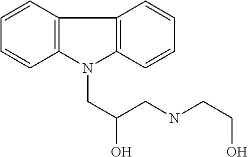 | C 71.81% H 7.09% N 9.85% O 11.25% | C17H20N2O2 | 284.36115 | ChemBridge | 2 |
| 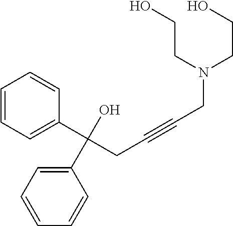 | C 74.31% H 7.42% N 4.13% O 14.14% | C21H25NO3 | 339.4383 | ChemBridge | 2 |
| 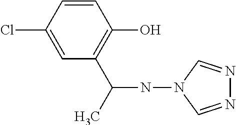 | C 50.32% H 4.65% Cl 14.85% N 23.47% O 6.70% | C10H11ClN4O | 238.67837 | ChemBridge | 2 |
| 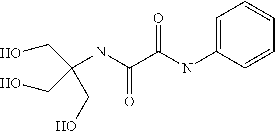 | C 53.73% H 6.01% N 10.44% O 29.82% | C12H16N2O5 | 268.27172 | ChemBridge | 2 |
| 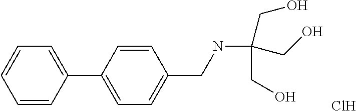 | C 63.06% H 6.85% Cl 10.95% N 4.33% O 14.82% | C17H22ClNO3 | 323.82279 | ChemBridge | 2 |
| 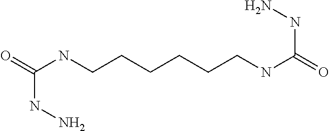 | C 41.37% H 8.68% N 36.18% O 13.78% | C8H20N6O2 | 232.2876 | Timtec | 2 |
| 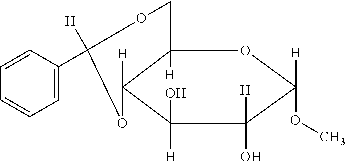 | C 59.57% H 6.43% O 34.01% | C14H18O6 | 282.29596 | Timtec | 2 |
| 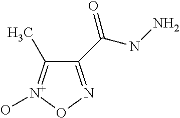 | C 30.39% H 3.83% N 35.43% O 30.36% | C4H6N4O3 | 158.11742 | Timtec | 2 |
| 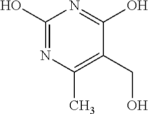 | C 46.15% H 5.16% N 17.94% O 30.74% | C6H8N2O3 | 156.14226 | Timtec | 2 |
| 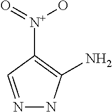 | C 28.13% H 3.15% N 43.74% O 24.98% | C3H4N4O2 | 128.09093 | Timtec | 2 |
| 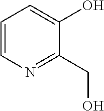 | C 57.59% H 5.64% N 11.19% O 25.57% | C6H7NO2 | 125.12819 | Timtec | 2 |
| 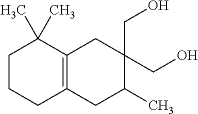 | C 75.58% H 10.99% O 13.42% | C15H26O2 | 238.37327 | Timtec | 2 |
| 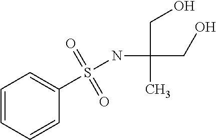 | C 48.97% H 6.16% N 5.71% O 26.09% S 13.07% | C10H15NO4S | 245.29935 | Timtec | 2 |
| 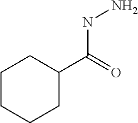 | C 59.13% H 9.92% N 19.70% O 11.25% | C7H14N2O | 142.20243 | Timtec | 2 |
| 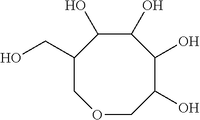 | C 46.15% H 7.75% O 46.10% | C8H16O6 | 208.21312 | Timtec | 2 |
| 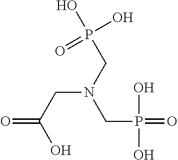 | C 18.26% H 4.21% N 5.32% O 48.65% P 23.55% | C4H11NO8P2 | 263.08177 | Timtec | 2 |
| 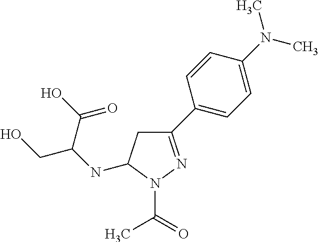 | C 57.47% H 6.63% N 16.76% O 19.14% | C16H22N4O4 | 334.37814 | Timtec | 2 |
| 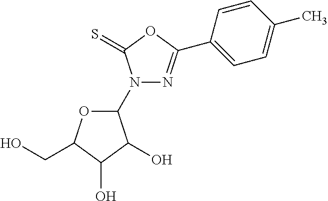 | C 51.84% H 4.97% N 8.64% O 24.66% S 9.89% | C14H16N2O5S | 324.35802 | Timtec | 2 |
| 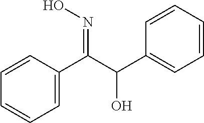 | C 73.99% H 5.77% N 6.16% O 14.08% | C14H13NO2 | 227.26521 | Timtec | 2 |
| 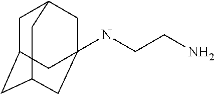 | C 74.17% H 11.41% N 14.42% | C12H22N2 | 194.32254 | Timtec | 2 |
| 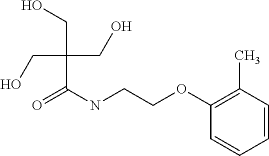 | C 59.35% H 7.47% N 4.94% O 28.23% | C14H21NO5 | 283.32717 | Timtec | 2 |
| 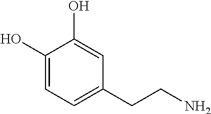 | C 62.73% H 7.24% N 9.14% O 20.89% | C8H11NO2 | 153.18237 | Timtec | 2 |
| 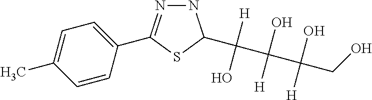 | C 52.33% H 6.08% N 9.39% O 21.45% S 10.75% | C13H18N2O4S | 298.36341 | Timtec | 2 |
| 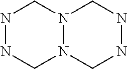 | C 33.32% H 8.39% N 58.29% | C4H12N6 | 144.18044 | Timtec | 2 |
| 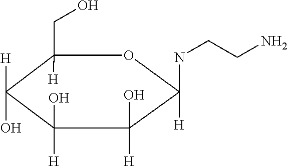 | C 43.24% H 8.16% N 12.60% O 36.00% | C8H18N2O5 | 222.24306 | Timtec | 2 |
| 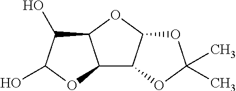 | C 49.54% H 6.47% O 43.99% | C9H14O6 | 218.20833 | Timtec | 2 |
| 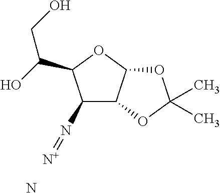 | C 44.08% H 6.17% N 17.13% O 32.62% | C9H15N3O5 | 245.237 | Timtec | 2 |
| 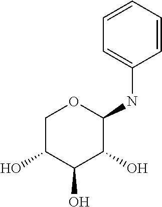 | C 58.66% H 6.71% N 6.22% O 28.41% | C11H15NO4 | 225.2465 | Timtec | 1 |
| 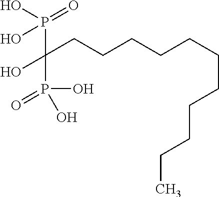 | C 41.62% H 8.15% O 32.34% P 17.89% | C12H28O7P2 | 346.30036 | Timtec | 1 |
| 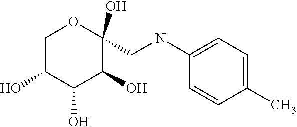 | C 57.98% H 7.11% N 5.20% O 29.71% | C13H19NO5 | 269.30008 | Timtec | 1 |
| 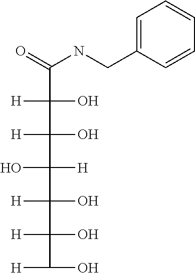 | C 53.33% H 6.71% N 4.44% O 35.52% | C14H21NO7 | 315.32597 | Timtec | 1 |
| 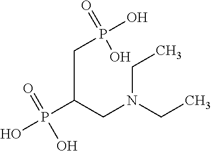 | C 30.55% H 6.96% N 5.09% O 34.88% P 22.51% | C7H19NO6P2 | 275.18018 | Timtec | 1 |
| 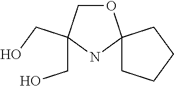 | C 57.73% H 9.15% N 7.48% O 25.63% | C9H17NO3 | 187.24074 | Timtec | 1 |
| 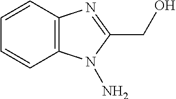 | C 58.89% H 5.56% N 25.75% O 9.80% | C8H9N3O | 163.18043 | Timtec | 1 |
| 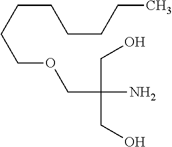 | C 61.77% H 11.66% N 6.00% O 20.57% | C12H27NO3 | 233.35389 | Timtec | 1 |
| 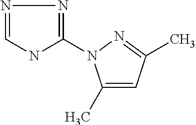 | C 51.52% H 5.56% N 42.92% | C7H9N5 | 163.18328 | Timtec | 1 |
| 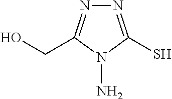 | C 24.65% H 4.14% N 38.33% O 10.95% S 21.94% | C3H6N4OS | 146.17147 | Timtec | 1 |
| 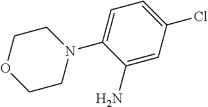 | C 56.47% H 6.16% Cl 16.67% N 13.17% O 7.52% | C10H13ClN2O | 212.68091 | Timtec | 1 |
| 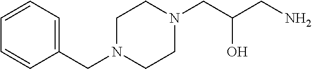 | C 67.44% H 9.30% N 16.85% O 6.42% | C14H23N3O | 249.35891 | Timtec | 1 |
| 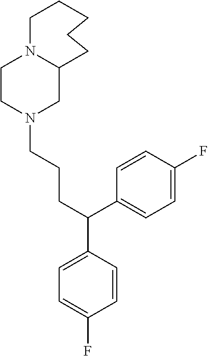 | C 75.34% H 8.09% F 9.53% N 7.03% | C25H32F2N2 | 398.54399 | Timtec | 1 |
| 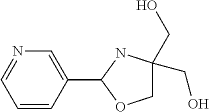 | C 57.13% H 6.71% N 13.32% O 22.83% | C10H14N2O3 | 210.23468 | Timtec | 1 |
| 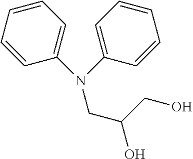 | C 74.05% H 7.04% N 5.76% O 13.15% | C15H17NO2 | 243.30824 | Timtec | 1 |
| 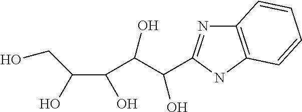 | C 53.73% H 6.01% N 10.44% O 29.82% | C12H16N2O5 | 268.27172 | Timtec | 1 |
| 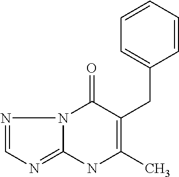 | C 64.99% H 5.03% N 23.32% O 6.66% | C13H12N4O | 240.26679 | Timtec | 1 |
| 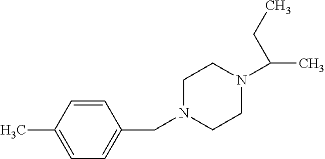 | C 77.99% H 10.64% N 11.37% | C16H26N2 | 246.39902 | Timtec | 1 |
| 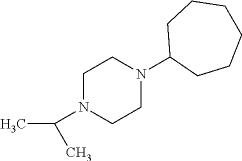 | C 74.94% H 12.58% N 12.48% | C14H28N2 | 224.39266 | Timtec | 1 |
| 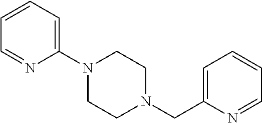 | C 70.84% H 7.13% N 22.03% | C15H18N4 | 254.33751 | Timtec | 1 |
| 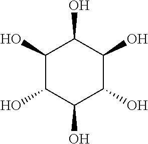 | C 40.00% H 6.71% O 53.28% | C6H12O6 | 180.15894 | Sigma- Aldrich | 34 |
| 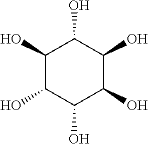 | C 40.00% H 6.71% O 53.28% | C6H12O6 | 180.15894 | Sigma- Aldrich | 2 |
| 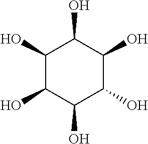 | C 40.00% H 6.71% O 53.28% | C6H12O6 | 180.15894 | J. McLaurin | 42 |
| 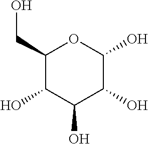 | C 40.00% H 6.71% O 53.28% | C6H12O6 | 180.15894 | Merck | 2 |
| 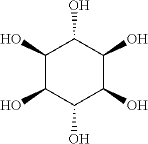 | C 40.00% H 6.71% O 53.28% | C6H12O6 | 180.15894 | Sigma- Aldrich | 4 |
| 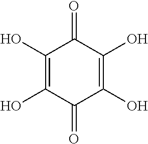 | C 41.88% H 2.34% O 55.78% | C6H4O6 | 172.09518 | Sigma- Aldrich | 3 |
| 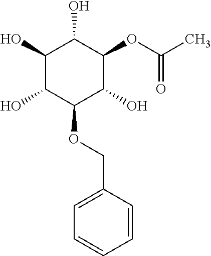 | C 57.69% H 6.45% O 35.86% | C15H20O7 | 312.32245 | Dalton | 1 |
| 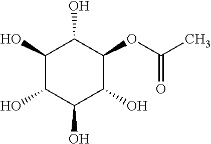 | C 43.25% H 6.35% O 50.40% | C8H14O7 | 222.19658 | Dalton | 31 |
| 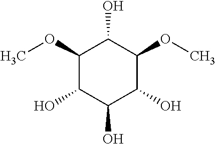 | C 46.15% H 7.75% O 46.10% | C8H16O6 | 208.21312 | Dalton | 3 |
| 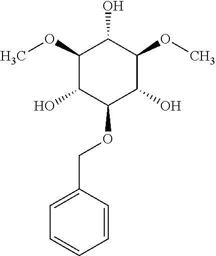 | C 60.39% H 7.43% O 32.18% | C15H22O6 | 298.33899 | Dalton | 2 |
| 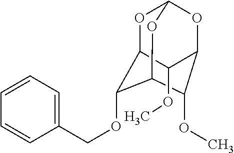 | C 62.33% H 6.54% O 31.13% | C16H20O6 | 308.3342 | Dalton | 1 |
| 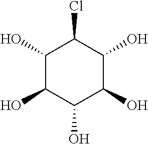 | C 36.29% H 5.58% Cl 17.85% O 40.28% | C6H11ClO5 | 198.60457 | V-Labs | 67 |
| 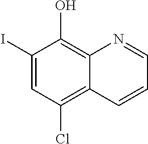 | C 35.38% H 1.65% Cl 11.60% I 41.54% N 4.58% O 5.24% | C9H5ClINO | 305.5037 | Sigma- Aldrich | 2 |

1. A method for screening a compound library to determine the relative or absolute affinity of a plurality of putative modulators to at least one Amyloid target comprising:
(a) providing a compound library comprising a plurality of putative modulators to at least one Amyloid target;
(b) applying the compound library to a column comprising at least one Amyloid target, wherein the at least one Amyloid target is optionally bound to a solid phase support, under frontal affinity chromatography conditions to provide an effluent;
(c) continuously or intermittently applying the effluent to a mass spectrometer to provide mass spectra of the constituent putative modulators present in the effluent; and
(d) evaluating the mass spectra to determine a breakthrough time for the putative modulators.

2. A method according to claim 1, further comprising: (e) determining an affinity to the at least one Amyloid target for a putative modulator in the compound library relative to another putative modulator in the library by comparing the breakthrough time for the putative modulator to the breakthrough time for the other putative modulator.

3. A method according to claim 2, further comprising: (f) determining a dissociation constant, K_{d}, for a putative modulator in the compound library and the at least one Amyloid target.

4. (canceled)

5. (canceled)

6. A method for screening a compound library for putative modulators that interfere with the interaction of an indicator agent and at least one Amyloid target or breaks down the at least one Amyloid target, comprising:
(a) providing a compound library comprising a plurality of putative modulators,
(b) continuously applying the compound library to a column comprising at least one Amyloid target, wherein the at least one Amyloid target is optionally immobilized, under frontal affinity chromatography conditions to equilibrate the column with the compound library;
(c) providing at least one indicator agent having a pre-determined affinity for the at least one Amyloid target, and having a pre-determined breakthrough time on the column in the absence of the compound library;
(d) continuously applying (i) a mixture comprising the compound library and the at least one indicator agent, or (ii) the at least one indicator agent, to the column under frontal affinity chromatography conditions to provide an effluent;
(e) analyzing the effluent by mass spectrometry to determine a breakthrough time for the at least one indicator agent in the presence and absence of the compound library; and
(f) determining whether any putative modulators interfere with the interaction or binding of the at least one indicator agent to the at least one Amyloid target or breaks down the at least one Amyloid target, by comparing the breakthrough time for the at least one indicator agent in the presence of the compound library with the pre-determined breakthrough time for the at least one indicator agent in the absence of the compound library.

7. A method according to claim 6, wherein the putative modulator shifts the breakthrough time of the at least one indicator agent by at least 5% to 10%.

8. (canceled)

9. (canceled)

10. A method for screening a compound library to determine the relative affinity of a plurality of putative modulators to an Amyloid target relative to an indicator agent having a pre-determined affinity for the Amyloid target, comprising:
(a) providing a compound library comprising a plurality of putative modulators;
(b) providing at least one void marker compound;
(c) providing a column comprising an Amyloid target, wherein the Amyloid target is optionally bound to a solid phase support;
(d) providing an indicator agent having a pre-determined affinity for the Amyloid target and having a pre-determined breakthrough time on the column in the absence of the compound library relative to the at least one void marker compound and having a predetermined signal intensity in the presence of the compound library;
(e) applying a mixture comprising the compound library and the indicator agent to the column under frontal affinity chromatography conditions to provide an effluent; and
(f) analyzing the effluent to determine a breakthrough time and/or signal intensity for the indicator agent.

11. A method according to claim 10, further comprising: (g) determining whether any putative modulators of the compound library have an affinity for the Amyloid target by comparing the breakthrough time for the indicator agent from step (f) with the pre-determined breakthrough time for the indicator agent in the absence of the compound library.

12. A method according to claim 11, further comprising: (h) determining whether the affinity for the Amyloid target is due to the plurality of modulators having weaker affinity for the Amyloid target relative to the indicator agent or to the plurality of modulators having stronger affinity for the Amyloid target relative to the indicator agent by comparing the signal intensity of the indicator agent in the effluent with the pre-determined signal intensity for the indicator agent.

13. A method according to claim 1, wherein the Amyloid target is an Aβ target.

14. A method according to claim 13, wherein the Aβ target comprises Aβ fibrils.

15. A method according to claim 1, wherein the at least one Amyloid target is bound to a solid phase support.

16. (canceled)

17. A method according to claim 6, wherein the at least one indicator agent is β-amyloid monomer.

18. A method according to claim 17, wherein the at least one indicator agent is an Aβ1-42 monomer.

19. A method according to claim 6, wherein the pre-determined breakthrough time for the at least one indicator agent in the absence of the compound library is less than about 5 minutes.

20. (canceled)

21. (canceled)

22. A method according to claim 1, wherein the putative modulators are carbohydrates, monosaccharides, oligosaccharides, polysaccharides, amino acids, peptides, oligopeptides, polypeptides, proteins, nucleosides, nucleotides, oligonucleotides, polynucleotides, lipids, retinoids, steroids, glycopeptides, glycoproteins, glycolipids, proteoglycans, synthetic analogs thereof, or derivatives thereof.

23. A method according to claim 22, wherein the putative modulators are synthetic small molecule organic compounds.

24. A method according to claim 22, wherein the putative modulators are natural products.

25. A method according to claim 1, wherein the mass spectrometer is an electrospray mass spectrometer.

26. A method according to claim 1, further comprising determining the structure of the putative modulator identified according to the method.

27. A modulator of the Amyloid target identified according to a method of claim 6.
